SEQUENCE LISTING (82 sequences)

SEQ ID NO: 1 - protein, 1369 aa, Artificial Sequence
  DHX29
MGGKNKKHKAPAAAVVRAAVSASRAKSAEAGIAGEAQSKKPVSRPATAAAAAAGSREPRVKQGPKIYSFNSTNDSSGPANLDKSILKVVINNKLEQRIIGVINEHKKQNNDKGMISGRLTAKKLQDLYMALQAFSFKTKDIEDAMTNTLLYGGDLHSALDWLCLNLSDDALPEGFSQEFEEQQPKSRPKFQSPQIQATISPPLQPKTKTYEEDPKSKPKKEEKNMEVNMKEWILRYAEQQNEEEKNENSKSLEEEEKFDPNERYLHLAAKLLDAKEQAATFKLEKNKQGQKEAQEKIRKFQREMETLEDHPVFNPAMKISHQQNERKKPPVATEGESALNFNLFEKSAAATEEEKDKKKEPHDVRNFDYTARSWTGKSPKQFLIDWVRKNLPKSPNPSFEKVPVGRYWKCRVRVIKSEDDVLVVCPTILTEDGMQAQHLGATLALYRLVKGQSVHQLLPPTYRDVWLEWSDAEKKREELNKMETNKPRDLFIAKLLNKLKQQQQQQQQHSENKRENSEDPEESWENLVSDEDFSALSLESANVEDLEPVRNLFRKLQSTPKYQKLLKERQQLPVFKHRDSIVETLKRHRVVVVAGETGSGKSTQVPHFLLEDLLLNEWEASKCNIVCTQPRRISAVSLANRVCDELGCENGPGGRNSLCGYQIRMESRACESTRLLYCTTGVLLRKLQEDGLLSNVSHVIVDEVHERSVQSDFLLIILKEILQKRSDLHLILMSATVDSEKFSTYFTHCPILRISGRSYPVEVFHLEDIIEETGFVLEKDSEYCQKFLEEEEEVTINVTSKAGGIKKYQEYIPVQTGAHADLNPFYQKYSSRTQHAILYMNPHKINLDLILELLAYLDKSPQFRNIEGAVLIFLPGLAHIQQLYDLLSNDRRFYSERYKVIALHSILSTQDQAAAFTLPPPGVRKIVLATNIAETGITIPDVVFVIDTGRTKENKYHESSQMSSLVETFVSKASALQRQGRAGRVRDGFCFRMYTRERFEGFMDYSVPEILRVPLEELCLHIMKCNLGSPEDFLSKALDPPQLQVISNAMNLLRKIGACELNEPKLTPLGQHLAALPVNVKIGKMLIFGAIFGCLDPVATLAAVMTEKSPFTTPIGRKDEADLAKSALAMADSDHLTIYNAYLGWKKARQEGGYRSEITYCRRNFLNRTSLLTLEDVKQELIKLVKAAGFSSSTTSTSWEGNRASQTLSFQEIALLKAVLVAGLYDNVGKIIYTKSVDVTEKLACIVETAQGKAQVHPSSVNRDLQTHGWLLYQEKIRYARVYLRETTLITPFPVLLFGGDIEVQHRERLLSIDGWIYFQAPVKIAVIFKQLRVLIDSVLRKKLENPKMSLENDKILQIITELIKTENN

SEQ ID NO: 2 - protein, 295 aa, Artificial Sequence [sequence omitted]

SEQ ID NO: 3 - protein, 293 aa, Artificial Sequence [sequence omitted]

SEQ ID NO: 4 - protein, 243 aa, Artificial Sequence
  Ribosomal protein rpS3
MAVQISKKRKFVADGIFKAELNEFLTRELAEDGYSGVEVRVTPTRTEIIILATRTQNVLGEKGRRIRELTAVVQKRFGFPEGSVELYAEKVATRGLCAIAQAESLRYKLLGGLAVRRACYGVLRFIMESGAKGCEVVVSGKLRGQRAKSMKFVDGLMIHSGDPVNYYVDTAVRHVLLRQGVLGIKVKIMLPWDPTGKIGPKKPLPDHVSIVEPKDEILPTTPISEQKGGKPEPPAMPQPVPTA

SEQ ID NO: 5 - protein, 264 aa, Artificial Sequence [sequence omitted]

SEQ ID NO: 6 - protein, 263 aa, Artificial Sequence [sequence omitted]

SEQ ID NO: 7 - protein, 204 aa, Artificial Sequence [sequence omitted]

SEQ ID NO: 8 - protein, 249 aa, Artificial Sequence
  Ribosomal protein rpS6
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTKKNKEEAAEYAKLLAKRMKEAKEKRQEQIAKRRRLSSLRASTSKSESSQK

SEQ ID NO: 9 - protein, 194 aa, Artificial Sequence [sequence omitted]

SEQ ID NO: 10 - protein, 208 aa, Artificial Sequence [sequence omitted]

SEQ ID NO: 11 - protein, 194 aa, Artificial Sequence [sequence omitted]

SEQ ID NO: 12 - protein, 165 aa, Artificial Sequence
  Ribosomal protein rpS10
MLMPKKNRIAIYELLFKEGVMVAKKDVHMPKHPELADKNVPNLHVMKAMQSLKSRGYVKEQFAWRHFYWYLTNEGIQYLRDYLHLPPEIVPATLRRSRPETGRPRPKGLEGERPARLTRGEADRDTYRRSAVPPGADKKAEAGAGSATEFQFRGGFGRGRGQPPQ

SEQ ID NO: 13 - protein, 158 aa, Artificial Sequence [sequence omitted]

SEQ ID NO: 14 - protein, 132 aa, Artificial Sequence [sequence omitted]

SEQ ID NO: 15 - protein, 151 aa, Artificial Sequence [sequence omitted]

SEQ ID NO: 16 - protein, 150 aa, Artificial Sequence
  Ribosomal protein rpS14
MAPRKGKEKKEEQVISLGPQVAEGENVFGVCHIFASFNDTFVHVTDLSGKETICRVTGGMKVKADRDESSPYAAMLAAQDVAQRCKELGITALHIKLRATGGNRTKTPGPGAQSALRALARSGMKIGRIEDVTPIPSDSTRRKGGRRGRR

SEQ ID NO: 17 - protein, 145 aa, Artificial Sequence [sequence omitted]

SEQ ID NO: 18 - protein, 130 aa, Artificial Sequence [sequence omitted]

SEQ ID NO: 19 - protein, 146 aa, Artificial Sequence [sequence omitted]

SEQ ID NO: 20 - protein, 135 aa, Artificial Sequence [sequence omitted]

SEQ ID NO: 21 - protein, 152 aa, Artificial Sequence [sequence omitted]

SEQ ID NO: 22 - protein, 145 aa, Artificial Sequence [sequence omitted]

SEQ ID NO: 23 - protein, 119 aa, Artificial Sequence [sequence omitted]

SEQ ID NO: 24 - protein, 83 aa, Artificial Sequence
  Ribosomal protein rpS21
MQNDAGEFVDLYVPRKCSASNRIIGAKDHASIQMNVAEVDKVTGRFNGQFKTYAICGAIRRMGESDDSILRLAKADGIVSKNF

SEQ ID NO: 25 - protein, 143 aa, Artificial Sequence [sequence omitted]

SEQ ID NO: 26 - protein, 133 aa, Artificial Sequence [sequence omitted]

SEQ ID NO: 27 - protein, 125 aa, Artificial Sequence [sequence omitted]

SEQ ID NO: 28 - protein, 115 aa, Artificial Sequence [sequence omitted]

SEQ ID NO: 29 - protein, 84 aa, Artificial Sequence
  Ribosomal protein rpS27
MPLAKDLLHPSPEEEKRKHKKKRLVQSPNSYFMDVKCPGCYKITTVFSHAQTVVLCVGCSTVLCQPTGGKARLTEGCSFRRKQH

SEQ ID NO: 30 - protein, 80 aa, Artificial Sequence [sequence omitted]

SEQ ID NO: 31 - protein, 69 aa, Artificial Sequence [sequence omitted]

SEQ ID NO: 32 - protein, 56 aa, Artificial Sequence [sequence omitted]

SEQ ID NO: 33 - protein, 59 aa, Artificial Sequence [sequence omitted]

SEQ ID NO: 34 - DNA, 1869 bp, Artificial Sequence
  H. sapiens 18S
tacctggttgatcctgccagtagcatatgcttgtctcaaagattaagccatgcatgtctgagtacgcacggccggtacagtgaaactgcgaatggctcattaaatcagttatggttcctttggtcgctcgctcctctcctacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

SEQ ID NO: 35 - protein, 13 aa, Artificial Sequence
  Truncated peptide from DHX29 (251-263)
SLEEEEKFDPNER

SEQ ID NO: 36 - protein, 8 aa, Artificial Sequence
  Truncated peptide from DHX29 (394-401)
SPNPSFEK

SEQ ID NO: 37 - protein, 6 aa, Artificial Sequence
  Truncated peptide from DHX29 (489-494)
DLFIAK

SEQ ID NO: 38 - protein, 12 aa, Artificial Sequence
  Truncated peptide from DHX29 (590-601)
VVVVAGETGSGK

SEQ ID NO: 39 - protein, 14 aa, Artificial Sequence
  Truncated peptide from DHX29 (1204-1217)
ASQTLSFQEIALLK

SEQ ID NO: 40 - protein, 11 aa, Artificial Sequence
  Truncated peptide from DHX29 (1243-1253)
LACIVETAQGK

SEQ ID NO: 41 - protein, 8 aa, Artificial Sequence
  Truncated peptide from DHX29 (1334-1341)
VLIDSVLR

SEQ ID NO: 42 - protein, 10 aa, Artificial Sequence
  Truncated peptide from DHX29 (1356-1365)
ILQIITELIK

SEQ ID NO: 43 - protein, 9 aa, Artificial Sequence
  Truncated peptide from DHX29 (98-106)
IIGVINEHK

SEQ ID NO: 44 - protein, 13 aa, Artificial Sequence
  Truncated peptide from DHX29 (251-263)
SLEEEEKFDPNER

SEQ ID NO: 45 - protein, 12 aa, Artificial Sequence
  Truncated peptide from DHX29 (590-601)
VVVVAGETGSGK

SEQ ID NO: 46 - protein, 14 aa, Artificial Sequence
  Truncated peptide from DHX29 (642-655)
VCDELGCENGPGGR

SEQ ID NO: 47 - protein, 9 aa, Artificial Sequence
  Truncated peptide from DHX29 (656-664)
NSLCGYQIR

SEQ ID NO: 48 - RNA, 589 nt, Artificial Sequence
  Messenger RNA for beta-globin
acacuugcuuuugacacaacuguguuuacuugcaaucccccaaaacagacagaauggugcaucuguccagugaggagaagucugcggucacugcccuguggggcaaggugaauguggaagaaguugguggugaggcccugggcaggcugcugguugucuacccauggacccagagguucuucgaguccuuuggggaccuguccucugcaaaugcuguuaugaacaauccuaaggugaaggcucauggcaagaaggugcuggcugccuucagugagggucugagucaccuggacaaccucaaaggcaccuuugcuaagcugagugaacugcacugugacaagcugcacguggauccugagaacuucaggcuccugggcaacgugcugguuauugugcugucucaucauuuuggcaaagaauucacuccucaggugcaggcugccuaucagaaggugguggcugguguggccaaugcccuggcucacaaauaccacugagaucuuuuucccucugccaaaaauuauggggacaucaugaagccccuugagcaucugacuucuggcuaauaaaggaaauuuauuuucauugc

SEQ ID NO: 49 - protein, 406 aa, Artificial Sequence
  Eukaryotic translation initiation factor 4A isoform 1 [Homo sapiens]
MSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLI

SEQ ID NO: 50 - protein, 611 aa, Artificial Sequence
  Eukaryotic translation initiation factor 4B [Homo sapiens]
MAASAKKKNKKGKTISLTDFLAEDGGTGGGSTYVSKPVSWADETDDLEGDVSTTWHSNDDDVYRAPPIDRSILPTAPRAAREPNIDRSRLPKSPPYTAFLGNLPYDVTEESIKEFFRGLNISAVRLPREPSNPERLKGFGYAEFEDLDSLLSALSLNEESLGNRRIRVDVADQAQDKDRDDRSFGRDRNRDSDKTDTDWRARPATDSFDDYPPRRGDDSFGDKYRDRYDSDRYRDGYRDGYRDGPRRDMDRYGGRDRYDDRGSRDYDRGYDSRIGSGRRAFGSGYRRDDDYRGGGDRYEDRYDRRDDRSWSSRDDYSRDDYRRDDRGPPQRPKLNLKPRSTPKEDDSSASTSQSTRAASIFGGAKPVDTAAREREVEERLQKEQEKLQRQLDEPKLERRPRERHPSWRSEETQERERSRTGSESSQTGTSTTSSRNARRRESEKSLENETLNKEEDCHSPTSKPPKPDQPLKVMPAPPPKENAWVKRSSNPPARSQSSDTEQQSPTSGGGKVAPAQPSEEGPGRKDENKVDGMNAPKGQTGNSSRGPGDGGNRDHWKESDRKDGKKDQDSRSAPEPKKPEENPASKFSSASKYAALSVDGEDENEGEDYAE

SEQ ID NO: 51 - protein, 217 aa, Artificial Sequence [sequence omitted]

SEQ ID NO: 52 - protein, 1599 aa, Artificial Squence
  Eukaryotic translation initiation factor 4G1 isoform 1 [Homo sapiens]
MNKAPQSTGPPPAPSPGLPQPAFPPGQTAPVVFSTPQATQMNTPSQPRQHFYPSRAQPPSSAASRVQSAAPARPGPAAHVYPAGSQVMMIPSQISYPASQGAYYIPGQGRSTYVVPTQQYPVQPGAPGFYPGASPTEFGTYAGAYYPAQGVQQFPTGVAPAPVLMNQPPQIAPKRERKTIRIRDPNQGGKDITEEIMSGARTASTPTPPQTGGGLEPQANGETPQVAVIVRPDDRSQGAIIADRPGLPGPEHSPSESQPSSPSPTPSPSPVLEPGSEPNLAVLSIPGDTMTTIQMSVEESTPISRETGEPYRLSPEPTPLAEPILEVEVTLSKPVPESEFSSSPLQAPTPLASHTVEIHEPNGMVPSEDLEPEVESSPELAPPPACPSESPVPIAPTAQPEELLNGAPSPPAVDLSPVSEPEEQAKEVTASMAPPTIPSATPATAPSATSPAQEEEMEEEEEEEEGEAGEAGEAESEKGGEELLPPESTPIPANLSQNLEAAAATQVAVSVPKRRRKIKELNKKEAVGDLLDAFKEANPAVPEVENQPPAGSNPGPESEGSGVPPRPEEADETWDSKEDKIHNAENIQPGEQKYEYKSDQWKPLNLEEKKRYDREFLLGFQFIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 53 - DNA, 75 bp, Artificial Sequence
  Human initiator Met-tRNA-I
agcagagtggcgcagcggaagcgtgctgggcccataacccagaggtcgatggatcgaaaccatcctctgctacca

SEQ ID NO: 54 - protein, 113 aa, Artificial Sequence
  Eukaryotic translation initiation factor 1
MSAIQNLHSFDPFADASKGDDLLPAGTEDYIHIRIQQRNGRKTLTTVQGIADDYDKKKLVKAFKKKFACNGTVIEHPEYGEVIQLQGDQRKNICQFLVEIGLAKDDQLKVHGF

SEQ ID NO: 55 - protein, 144 aa, Artificial Sequence
  Eukaryotic translation initiation factor 1A
MPKNKGKGGKNRRRGKNENESEKRELVFKEDGQEYAQVIKMLGNGRLEAMCFDGVRRLCHIRGKLRKKVWINTSDIILIGLRDYQDNKADVILKYNADEARSLKAYGELPEHAKINETDTFGPGDDDEIQFDDIGDDDEDIDDI

SEQ ID NO: 56 - protein, 315 aa, Artificial Sequence
  Eukaryotic translation initiation factor 2, subunit 1 alpha [Homo sapiens]
MPGLSCRFYQHKFPEVEDVVMVNVRSIAEMGAYVSLLEYNNIEGMILLSELSRRRIRSINKLIRIGRNECVVVIRVDKEKGYIDLSKRRVSPEEAIKCEDKFTKSKTVYSILRHVAEVLEYTKDEQLESLFQRTAWVFDDKYKRPGYGAYDAFKHAVSDPSILDSLDLNEDEREVLINNINRRLTPQAVKIRADIEVACYGYEGIDAVKEALRAGLNCSTENMPIKINLIAPPRYVMTTTTLERTEGLSVLSQAMAVIKEKIEEKRGVFNVQMEPKVVTDTDETELARQMERLERENAEVDGDDDAEEMEAKAED

SEQ ID NO: 57 - protein, 333 aa, Artificial Sequence
  Eukaryotic translation initiation factor 2 beta [Homo sapiens]
MSGDEMIFDPTMSKKKKKKKKPFMLDEEGDTQTEETQPSETKEVEPEPTEDKDLEADEEDTRKKDASDDLDDLNFFNQKKKKKKTKKIFDIDEAEEGVKDLKIESDVQEPTEPEDDLDIMLGNKKKKKKNVKFPDEDEILEKDEALEDEDNKKDDGISFSNQTGPAWAGSERDYTYEELLNRVFNIMREKNPDMVAGEKRKFVMKPPQVVRVGTKKTSFVNFTDICKLLHRQPKHLLAFLLAELGTSGSIDGNNQLVIKGRFQQKQIENVLRRYIKEYVTCHTCRSPDTILQKDTRLYFLQCETCHSRCSVASIKTGFQAVTGKRAQLRAKAN

SEQ ID NO: 58 - protein, 472 aa, Artificial Sequence
  Eukaryotic translation initiation factor 2, subunit 3 gamma [Homo sapiens]
MAGGEAGVTLGQPHLSRQDLTTLDVTKLTPLSHEVISRQATINIGTIGHVAHGKSTVVKAISGVHTVRFKNELERNITIKLGYANAKIYKLDDPSCPRPECYRSCGSSTPDEFPTDIPGTKGNFKLVRHVSFVDCPGHDILMATMLNGAAVMDAALLLIAGNESCPQPQTSEHLAAIEIMKLKHILILQNKIDLVKESQAKEQYEQILAFVQGTVAEGAPIIPISAQLKYNIEVVCEYIVKKIPVPPRDFTSEPRLIVIRSFDVNKPGCEVDDLKGGVAGGSILKGVLKVGQEIEVRPGIVSKDSEGKLMCKPIFSKIVSLFAEHNDLQYAAPGGLIGVGTKIDPTLCRADRMVGQVLGAVGALPEIFTELEISYFLLRRLLGVRTEGDKKAAKVQKLSKNEVLMVNIGSLSTGGRVSAVKADLGKIVLTNPVCTEVGEKIALSRRVEKHWRLIGWGQIRRGVTIKPTVDDD

SEQ ID NO: 59 - protein, 1382 aa, Artificial Sequence
  Eukaryotic translation initiation factor 3A [Homo sapiens]
MPAYFQRPENALKRANEFLEVGKKQPALDVLYDVMKSKKHRTWQKIHEPIMLKYLELCVDLRKSHLAKEGLYQYKNICQQVNIKSLEDVVRAYLKMAEEKTEAAKEESQQMVLDIEDLDNIQTPESVLLSAVSGEDTQDRTDRLLLTPWVKFLWESYRQCLDLLRNNSRVERLYHDIAQQAFKFCLQYTRKAEFRKLCDNLRMHLSQIQRHHNQSTAINLNNPESQSMHLETRLVQLDSAISMELWQEAFKAVEDIHGLFSLSKKPPKPQLMANYYNKVSTVFWKSGNALFHASTLHRLYHLSREMRKNLTQDEMQRMSTRVLLATLSIPITPERTDIARLLDMDGIIVEKQRRLATLLGLQAPPTRIGLINDMVRFNVLQYVVPEVKDLYNWLEVEFNPLKLCERVTKVLNWVREQPEKEPELQQYVPQLQNNTILRLLQQVSQIYQSIEFSRLTSLVPFVDAFQLERAIVDAARHCDLQVRIDHTSRTLSFGSDLNYATREDAPIGPHLQSMPSEQIRNQLTAMSSVLAKALEVIKPAHILQEKEEQHQLAVTAYLKNSRKEHQRILARRQTIEERKERLESLNIQREKEELEQREAELQKVRKAEEERLRQEAKEREKERILQEHEQIKKKTVRERLEQIKKTELGAKAFKDIDIEDLEELDPDFIMAKQVEQLEKEKKELQERLKNQEKKIDYFERAKRLEEIPLIKSAYEEQRIKDMDLWEQQEEERITTMQLEREKALEHKNRMSRMLEDRDLFVMRLKAARQSVYEEKLKQFEERLAEERHNRLEERKRQRKEERRITYYREKEEEEQRRAEEQMLKEREERERAERAKREEELREYQERVKKLEEVERKKRQRELEIEERERRREEERRLGDSSLSRKDSRWGDRDSEGTWRKGPEADSEWRRGPPEKEWRRGEGRDEDRSHRRDEERPRRLGDDEDREPSLRPDDDRVPRRGMDDDRGPRRGPEEDRFSRRGADDDRPSWRNTDDDRPPRRIADEDRGNWRHADDDRPPRRGLDEDRGSWRTADEDRGPRRGMDDDRGPRRGGADDERSSWRNADDDRGPRRGLDDDRGPRRGMDDDRGPRRGMDDDRGPRRGMDDDRGPRRGLDDDRGPWRNADDDRIPRRGAEDDRGPWRNMDDDRLSRRADDDRFPRRGDDSRPGPWRPLVKPGGWREKEKAREESWGPPRESRPSEEREWDREKERDRDNQDREENDKDPERERDRERDVDREDRFRRPRDEGGWRRGPAEESSSWRDSSRRDDRDRDDRRRERDDRRDLRERRDLRDDRDRRGPPLRSEREEVSSWRRADDRKDDRVEERDPPRRVPPPALSRDRERDRDREREGEKEKASWRAEKDRESLRRTKNETDEDGWTTVRR

SEQ ID NO: 60 - protein, 814 aa, Artificial Sequence
  Eukaryotic translation initiation factor 3B [Homo sapiens]
MQDAENVAVPEAAEERAEPGQQQPAAEPPPAEGLLRPAGPGAPEAAGTEASSEEVGIAEAGPESEVRTEPAAEAEAASGPSESPSPPAAEELPGSHAEPPVPAQGEAPGEQARDERSDSRAQAVSEDAGGNEGRAAEAEPRALENGDADEPSFSDPEDFVDDVSEEELLGDVLKDRPQEADGIDSVIVVDNVPQVGPDRLEKLKNVIHKIFSKFGKITNDFYPEEDGKTKGYIFLEYASPAHAVDAVKNADGYKLDKQHTFRVNLFTDFDKYMTISDEWDIPEKQPFKDLGNLRYWLEEAECRDQYSVIFESGDRTSIFWNDVKDPVSIEERARWTETYVRWSPKGTYLATFHQRGIALWGGEKFKQIQRFSHQGVQLIDFSPCERYLVTFSPLMDTQDDPQAIIIWDILTGHKKRGFHCESSAHWPIFKWSHDGKFFARMTLDTLSIYETPSMGLLDKKSLKISGIKDFSWSPGGNIIAFWVPEDKDIPARVTLMQLPTRQEIRVRNLFNVVDCKLHWQKNGDYLCVKVDRTPKGTQGVVTNFEIFRMREKQVPVDVVEMKETIIAFAWEPNGSKFAVLHGEAPRISVSFYHVKNNGKIELIKMFDKQQANTIFWSPQGQFVVLAGLRSMNGALAFVDTSDCTVMNIAEHYMASDVEWDPTGRYVVTSVSWWSHKVDNAYWLWTFQGRLLQKNNKDRFCQLLWRPRPPTLLSQEQIKQIKKDLKKYSKIFEQKDRLSQSKASKELVERRRTMMEDFRKYRKMAQELYMEQKNERLELRGGVDTDELDSNVDDWEEETIEFFVTEEIIPLGNQE

SEQ ID NO: 61 - protein, 913 aa, Artificial Sequence
  Eukaryotic translation initiation factor 3C [Homo sapiens]
MSRFFTTGSDSESESSLSGEELVTKPVGGNYGKQPLLLSEDEEDTKRVVRSAKDKRFEELTNLIRTIRNAMKIRDVTKCLEEFELLGKAYGKAKSIVDKEGVPRFYIRILADLEDYLNELWEDKEGKKKMNKNNAKALSTLRQKIRKYNRDFESHITSYKQNPEQSADEDAEKNEEDSEGSSDEDEDEDGVSAATFLKKKSEAPSGESRKFLKKMDDEDEDSEDSEDDEDWDTGSTSSDSDSEEEEGKQTALASRFLKKAPTTDEDKKAAEKKREDKAKKKHDRKSKRLDEEEEDNEGGEWERVRGGVPLVKEKPKMFAKGTEITHAVVIKKLNEILQARGKKGTDRAAQIELLQLLVQIAAENNLGEGVIVKIKFNIIASLYDYNPNLATYMKPEMWGKCLDCINELMDILFANPNIFVGENILEESENLHNADQPLRVRGCILTLVERMDEEFTKIMQNTDPHSQEYVEHLKDEAQVCAIIERVQRYLEEKGTTEEVCRIYLLRILHTYYKFDYKAHQRQLTPPEGSSKSEQDQAENEGEDSAVLMERLCKYIYAKDRTDRIRTCAILCHIYHHALHSRWYQARDLMLMSHLQDNIQHADPPVQILYNRTMVQLGICAFRQGLTKDAHNALLDIQSSGRAKELLGQGLLLRSLQERNQEQEKVERRRQVPFHLHINLELLECVYLVSAMLLEIPYMAAHESDARRRMISKQFHHQLRVGERQPLLGPPESMREHVVAASKAMKMGDWKTCHSFIINEKMNGKVWDLFPEADKVRTMLVRKIQEESLRTYLFTYSSVYDSISMETLSDMFELDLPTVHSIISKMIINEELMASLDQPTQTVVMHRTEPTAQQNLALQLAEKLGSLVENNERVFDHKQGTYGGYFRDQKDGYRKNEGYMRRGGYRQQQSQTAY

SEQ ID NO: 62 - protein, 548 aa, Artificial Sequence
  Eukaryotic translation initiation factor 3D [Homo sapiens]
MAKFMTPVIQDNPSGWGPCAVPEQFRDMPYQPFSKGDRLGKVADWTGATYQDKRYTNKYSSQFGGGSQYAYFHEEDESSFQLVDTARTQKTAYQRNRMRFAQRNLRRDKDRRNMLQFNLQILPKSAKQKERERIRLQKKFQKQFGVRQKWDQKSQKPRDSSVEVRSDWEVKEEMDFPQLMKMRYLEVSEPQDIECCGALEYYDKAFDRITTRSEKPLRSIKRIFHTVTTTDDPVIRKLAKTQGNVFATDAILATLMSCTRSVYSWDIVVQRVGSKLFFDKRDNSDFDLLTVSETANEPPQDEGNSFNSPRNLAMEATYINHNFSQQCLRMGKERYNFPNPNPFVEDDMDKNEIASVAYRYRRWKLGDDIDLIVRCEHDGVMTGANGEVSFINIKTLNEWDSRHCNGVDWRQKLDSQRGAVIATELKNNSYKLARWTCCALLAGSEYLKLGYVSRYHVKDSSRHVILGTQQFKPNEFASQINLSVENAWGILRCVIDICMKLEEGKYLILKDPNKQVIRVYSLPDGTFSSDEDEEEEEEEEEEEEEEET

SEQ ID NO: 63 - protein, 445 aa, Artificial Sequence
  Eukaryotic translation initiation factor 3E [Homo sapiens]
MAEYDLTTRIAHFLDRHLVFPLLEFLSVKEIYNEKELLQGKLDLLSDTNMVDFAMDVYKNLYSDDIPHALREKRTTVVAQLKQLQAETEPIVKMFEDPETTRQMQSTRDGRMLFDYLADKHGFRQEYLDTLYRYAKFQYECGNYSGAAEYLYFFRVLVPATDRNALSSLWGKLASEILMQNWDAAMEDLTRLKETIDNNSVSSPLQSLQQRTWLIHWSLFVFFNHPKGRDNIIDLFLYQPQYLNAIQTMCPHILRYLTTAVITNKDVRKRRQVLKDLVKVIQQESYTYKDPITEFVECLYVNFDFDGAQKKLRECESVLVNDFFLVACLEDFIENARLFIFETFCRIHQCISINMLADKLNMTPEEAERWIVNLIRNARLDAKIDSKLGHVVMGNNAVSPYQQVIEKTKSLSFRSQMLAMNIEKKLNQNSRSEAPNWATQDSGFY

SEQ ID NO: 64 - protein, 357 aa, Artificial Sequence
  Eukaryotic translation initiation factor 3F [Homo sapiens]
MATPAVPVSAPPATPTPVPAAAPASVPAPTPAPAAAPVPAAAPASSSDPAAAAAATAAPGQTPASAQAPAQTPAPALPGPALPGPFPGGRVVRLHPVILASIVDSYERRNEGAARVIGTLLGTVDKHSVEVTNCFSVPHNESEDEVAVDMEFAKNMYELHKKVSPNELILGWYATGHDITEHSVLIHEYYSREAPNPIHLTVDTSLQNGRMSIKAYVSTLMGVPGRTMGVMFTPLTVKYAYYDTERIGVDLIMKTCFSPNRVIGLSSDLQQVGGASARIQDALSTVLQYAEDVLSGKVSADNTVGRFLMSLVNQVPKIVPDDFETMLNSNINDLLMVTYLANLTQSQIALNEKLVNL

SEQ ID NO: 65 - protein, 320 aa, Artificial Sequence
  Eukaryotic translation initiation factor 3G [Homo sapiens]
MPTGDFDSKPSWADQVEEEGEDDKCVTSELLKGIPLATGDTSPEPELLPGAPLPPPKEVINGNIKTVTEYKIDEDGKKFKIVRTFRIETRKASKAVARRKNWKKFGNSEFDPPGPNVATTTVSDDVSMTFITSKEDLNCQEEEDPMNKLKGQKIVSCRICKGDHWTTRCPYKDTLGPMQKELAEQLGLSTGEKEKLPGELEPVQATQNKTGKYVPPSLRDGASRRGESMQPNRRADDNATIRVTNLSEDTRETDLQELFRPFGSISRIYLAKDKTTGQSKGFAFISFHRREDAARAIAGVSGFGYDHLILNVEWAKPSTN

SEQ ID NO: 66 - protein, 352 aa, Artificial Sequence
  Eukaryotic translation initiation factor 3H [Homo sapiens]
MASRKEGTGSTATSSSSTAGAAGKGKGKGGSGDSAVKQVQIDGLVVLKIIKHYQEEGQGTEVVQGVLLGLVVEDRLEITNCFPFPQHTEDDADFDEVQYQMEMMRSLRHVNIDHLHVGWYQSTYYGSFVTRALLDSQFSYQHAIEESVVLIYDPIKTAQGSLSLKAYRLTPKLMEVCKEKDFSPEALKKANITFEYMFEEVPIVIKNSHLINVLMWELEKKSAVADKHELLSLASSNHLGKNLQLLMDRVDEMSQDIVKYNTYMRNTSKQQQQKHQYQQRRQQENMQRQSRGEPPLPEEDLSKLFKPPQPPARMDSLLIAGQINTYCQNIKEFTAQNLGKLFMAQALQEYNN

SEQ ID NO: 67 - protein, 325 aa, Artificial Sequence
  Eukaryotic translation initiation factor 3I [Homo sapiens]
MKPILLQGHERSITQIKYNREGDLLFTVAKDPIVNVWYSVNGERLGTYMGHTGAVWCVDADWDTKHVLTGSADNSCRLWDCETGKQLALLKTNSAVRTCGFDFGGNIIMFSTDKQMGYQCFVSFFDLRDPSQIDNNEPYMKIPCNDSKITSAVWGPLGECIIAGHESGELNQYSAKSGEVLVNVKEHSRQINDIQLSRDMTMFVTASKDNTAKLFDSTTLEHQKTFRTERPVNSAALSPNYDHVVLGGGQEAMDVTTTSTRIGKFEARFFHLAFEEEFGRVKGHFGPINSVAFHPDGKSYSSGGEDGYVRIHYFDPQYFEFEFEA

SEQ ID NO: 68 - protein, 258 aa, Artificial Sequence
  Eukaryotic translation initiation factor 3J [Homo sapiens]
MAAAAAAAGDSDSWDADAFSVEDPVRKVGGGGTAGGDRWEGEDEDEDVKDNWDDDDDEKKEEAEVKPEVKISEKKKIAEKIKEKERQQKKRQEEIKKRLEEPEEPKVLTPEEQLADKLRLKKLQEESDLELAKETFGVNNAVYGIDAMNPSSRDDFTEFGKLLKDKITQYEKSLYYASFLEVLVRDVCISLEIDDLKKITNSLTVLCSEKQKQEKQSKAKKKKKGVVPGGGLKATMKDDLADYGGYDGGYVQDYEDFM

SEQ ID NO: 69 - protein, 218 aa, Artificial Sequence
  Eukaryotic translation initiation factor 3K [Homo sapiens]
MAMFEQMRANVGKLLKGIDRYNPENLATLERYVETQAKENAYDLEANLAVLKLYQFNPAFFQTTVTAQILLKALTNLPHTDFTLCKCMIDQAHQEERPIRQILYLGDLLETCHFQAFWQALDENMDLLEGITGFEDSVRKFICHVVGITYQHIDRWLLAEMLGDLSDSQLKVWMSKYGWSADESGQIFICSQEESIKPKNIVEKIDFDSVSSIMASSQ

SEQ ID NO: 70 - protein, 564 aa, Artificial Sequence
  Eukaryotic translation initiation factor 3L [Homo sapiens]
MSYPADDYESEAAYDPYAYPSDYDMHTGDPKQDLAYERQYEQQTYQVIPEVIKNFIQYFHKTVSDLIDQKVYELQASRVSSDVIDQKVYEIQDIYENSWTKLTERFFKNTPWPEAEAIAPQVGNDAVFLILYKELYYRHIYAKVSGGPSLEQRFESYYNYCNLFNYILNADGPAPLELPNQWLWDIIDEFIYQFQSFSQYRCKTAKKSEEEIDFLRSNPKIWNVHSVLNVLHSLVDKSNINRQLEVYTSGGDPESVAGEYGRHSLYKMLGYFSLVGLLRLHSLLGDYYQAIKVLENIELNKKSMYSRVPECQVTTYYYVGFAYLMMRRYQDAIRVFANILLYIQRTKSMFQRTTYKYEMINKQNEQMHALLAIALTMYPMRIDESIHLQLREKYGDKMLRMQKGDPQVYEELFSYSCPKFLSPVVPNYDNVHPNYHKEPFLQQLKVFSDEVQQQAQLSTIRSFLKLYTTMPVAKLAGFLDLTEQEFRIQLLVFKHKMKNLVWTSGISALDGEFQSASEVDFYIDKDMIHIADTKVARRYGDFFIRQIHKFEELNRTLKKMGQRP

SEQ ID NO: 71 - protein, 374 aa, Artificial Sequence
  Eukaryotic translation initiation factor 3M [Homo sapiens]
MSVPAFIDISEEDQAAELRAYLKSKGAEISEENSEGGLHVDLAQIIEACDVCLKEDDKDVESVMNSVVSLLLILEPDKQEALIESLCEKLVKFREGERPSLRLQLLSNLFHGMDKNTPVRYTVYCSLIKVAASCGAIQYIPTELDQVRKWISDWNLTTEKKHTLLRLLYEALVDCKKSDAASKVMVELLGSYTEDNASQARVDAHRCIVRALKDPNAFLFDHLLTLKPVKFLEGELIHDLLTIFVSAKLASYVKFYQNNKDFIDSLGLLHEQNMAKMRLLTFMGMAVENKEISFDTMQQELQIGADDVEAFVIDAVRTKMVYCKIDQTQRKVVVSHSTHRTFGKQQWQQLYDTLNAWKQNLNKVKNSLLSLSDT

SEQ ID NO: 72 - RNA, 2360 nt, Artificial Sequence
  (NCF2) 5′-UTR
ggaauucaacgcagaguacgcggggcaacacugagaaguuaucuuaagggaggcugggccccauucuacucaucuggcccagaaagugaacaccuugggggccacuaaggcagcccugcuaggggagacgcuccaaccugucuucucucugucuccuggcagcucucuuggccuccuaguuucuaccuaauccauggaagacgccaaaaacauaaagaaaggcccggcgccauucuauccgcuggaagauggaaccgcuggagagcaacugcauaaggcuaugaagagauacgcccugguuccuggaacaauugcuuuuacagaugcacauaucgagguggacaucacuuacgcugaguacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugggcauuucgcagccuaccgugguguucguuuccaaaaagggguugcaaaaaauuuugaacgugcaaaaaaagcucccaaucauccaaaaaauuauuaucauggauucuaaaacggauuaccagggauuucagucgauguacacguucgucacaucucaucuaccucccgguuuuaaugaauacgauuuugugccagaguccuucgauagggacaagacaauugcacugaucaugaacuccucuggaucuacuggucugccuaaaggugucgcucugccucauagaacugccugcgugagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuucugaggagccuucaggauuacaagauucaaagugcgcugcuggugccaacccuauucuccuucuucgccaaaagcacucugauugacaaauacgauuuaucuaauuuacacgaaauugcuucugguggcgcuccccucucuaaggaagucggggaagcgguugccaagagguuccaucugccagguaucaggcaaggauaugggcucacugagacuacaucagcuauucugauuacacccgagggggaugauaaaccgggcgcggucgguaaaguuguuccauuuuuugaagcgaagguuguggaucuggauaccgggaaaacgcugggcguuaaucaaagaggcgaacugugugugagagguccuaugauuauguccgguuauguaaacaauccggaagcgaccaacgccuugauugacaaggauggauggcuacauucuggagacauagcuuacugggacgaagacgaacacuucuucaucguugaccgccugaagucucugauuaaguacaaaggcuaucagguggcucccgcugaauuggaauccaucuugcuccaacaccccaacaucuucgacgcaggugucgcaggucuucccgacgaugacgccggugaacuucccgccgccguuguuguuuuggagcacggaaagacgaugacggaaaaagagaucguggauuacgucgccagucaaguaacaaccgcgaaaaaguugcgcggaggaguuguguuuguggacgaaguaccgaaaggucuuaccggaaaacucgacgcaagaaaaaucagagagauccucauaaaggccaagaagggcggaaagaucgccguguaauucuagagucggggcggccggccgcuucgagcagacaugauaagauacauugaugaguuuggacaaaccacaacuagaaugcagugaaaaaaaugcuuuauuugugaaauuugugaugcuauugcuuuauuuguaaccauuauaagcugcaauaaacaaguuaacaacaacaauugcauucauuuuauguuucagguucagggggaggugugggagguuuuuuaaagcaaguaaaaccucuacaaaugugguaaaaucgauaaggaucugaacgauggagcggagaaugggcggaacugggcggaguuaggggcgggaugggcggaguuaggggcgggacuaugguugcugacuaauugagaugcaugcuuugcauacuucugccugcuggggagccuggggacuuuccacaccugguugcugacuaauugagaugcaugcuuugcauacuucugccugcuggggagccuggggacuuuccacacccuaacugacacacauuccacagcggauc

SEQ ID NO: 73 - RNA, 2136 nt, Artificial Sequence
  CDC25 mRNA
gggcggccgcgaauucggucaacgccugcggcuguugauauucuugcucagaggccguaacuuuggccuucugcucagggaagacucugaguccgacguuggccuacccagucggaaggcagagcugcaaucuaguuaacuaccuccuuuccccuagauuuccuuucauucugcucaagucuucgccuguguccgaucccuaucuacuuucucuccucuuguaggcaagccucagacuccaggcuugagcuagguuuuguuuuucuccuggugagaauucgaagaccaugucuacggaacucuucucauccacaagagaggaaggaagcucuggcucaggacccaguuuuaggucuaaucaaaggaaaauguuaaaccugcuccuggagagagacacuuccuuuaccgucuguccagaugucccuagaacuccagugggcaaauuucuuggugauucugcaaaccuaagcauuuugucuggaggaaccccaaaacguugccucgaucuuucgaaucuuagcaguggggagauaacugccacucagcuuaccacuucugcagaccuugaugaaacuggucaccuggauucuucaggacuucaggaagugcauuuagcugggaugaaucaugaccagcaccuaaugaaauguagcccagcacagcuucuuuguagcacuccgaaugguuuggaccguggccauagaaagagagaugcaauguguaguucaucugcaaauaaagaaaaugacaauggaaacuugguggacagugaaaugaaauauuugggcagucccauuacuacuguuccaaaauuggauaaaaauccaaaccuaggagaagaccaggcagaagagauuucagaugaauuaauggaguuuucccugaaagaucaagaagcaaaggugagcagaaguggccuauaucgcuccccgucgaugccagagaacuugaacaggccaagacugaagcagguggaaaaauucaaggacaacacaauaccagauaaaguuaaaaaaaaguauuuuucuggccaaggaaagcucaggaagggcuuauguuuaaagaagacagucucucugugugacauuacuaucacucagaugcuggaggaagauucuaaccaggggcaccugauuggugauuuuuccaagguaugugcgcugccaaccgugucagggaaacaccaagaucugaaguaugucaacccagaaacaguggcugccuuacugucggggaaguuccagggucugauugagaaguuuuaugucauugauugucgcuauccauaugaguaucugggaggacacauccagggagccuuaaacuuauauagucaggaagaacuguuuaacuucuuucugaagaagcccaucgucccuuuggacacccagaagagaauaaucaucguguuccacugugaauucuccucagagaggggcccccgaaugugccgcugucugcgugaagaggacaggucucugaaccaguauccugcauuguacuacccagagcuauauauccuuaaaggcggcuacagagacuucuuuccagaauauauggaacugugugaaccacagagcuacugcccuaugcaucaucaggaccacaagacugaguugcugaggugucgaagccagagcaaagugcaggaaggggagcggcagcugcgggagcagauugcccuucuggugaaggacaugagcccaugauaacauuccagccacuggcugcuaacaagucaccaaaagacacugcagaaacccugagcagaaagaggccuucuggauggccaaacccaagauuauuaaaagaugucucugcaaaccaacaggcuaccaacuuguauccaggccugggaauggauuagguuucagcagagcugaaagcugguggcagaguccuggagcuggcucuauaaggcagccuugaguugcauagagauuuguauugguucagggaacucuggcauuccuuuucccaacuccucaugucuucucacaagccagccaacucuuucucucugggcuucgggcuaugcaagagcguugucuaccuucuuucuuuguauuuuccuucuuuguuucccccucuuucuuuuuuaaaaauggaaaaauaaacacuacagaaugaggucga

SEQ ID NO: 74 - RNA, 461 nt, Artificial Sequence
  CAA(Stem 3)-GUS mRNA
gcaacaacaacaacaacaacaacaacaacaacaacaacaacaagggcugcgggccuccgcagccccaacaacaaccaugguccguccuguagaaaccccaacccgugaaaucaaaaaacucgacggccugugggcauucagucuggaucgcgaaaacuguggaauugaucagcguuggugggaaagcgcguuacaagaaagccgggcaauugcugugccaggcaguuuuaacgaucaguucgccgaugcaauucguaauuaugcgggcaacgucugguaucagcgcgaagucuuuauaccgaugaaagguugggcaggccagcguaucgugcugcguuucgaugcggucacucauuacggcaaagugugggucaauaaucaggaagugauggagcaucagggcggcuauacgccauuugaagccgaugucacgccguauguuauugccgggaaaaguguac

SEQ ID NO: 75 - RNA, 469 nt, Artificial Sequence
  CAA(Stem 4)-GUS mRNA
gcaacaacaacaacaacaacaacaacaacaacaacaacaacaagggcugcgguggagccuuccaccgcagccccaacaacaaccaugguccguccuguagaaaccccaacccgugaaaucaaaaaacucgacggccugugggcauucagucuggaucgcgaaaacuguggaauugaucagcguuggugggaaagcgcguuacaagaaagccgggcaauugcugugccaggcaguuuuaacgaucaguucgccgaugcaauucguaauuaugcgggcaacgucugguaucagcgcgaagucuuuauaccgaugaaagguugggcaggccagcguaucgugcugcguuucgaugcggucacucauuacggcaaagugugggucaauaaucaggaagugauggagcaucagggcggcuauacgccauuugaagccgaugucacgccguauguuauugccgggaaaaguguac

SEQ ID NO: 76 - RNA, 453 nt, Artificial Sequence
  (CAA)n-GUS mRNA
gcaagaacaacaacaacaacaacaacaacaacaacaacaacaacaacaacaacaacaacaacaacaccaugguccguccuguagaaaccccaacccgugaaaucaaaaaacucgacggccugugggcauucagucuggaucgcgaaaacuguggaauugaucagcguuggugggaaagcgcguuacaagaaagccgggcaauugcugugccaggcaguuuuaacgaucaguucgccgaugcagauauucguaauuaugcgggcaacgucugguaucagcgcgaagucuuuauaccgaaagguugggcaggccagcguaucgugcugcguuucgaugcggucacucauuacggcaaagugugggucaauaaucaggaagugauggagcaucagggcggcuauacgccauuugaagccgaugucacgccguauguuauugccgggaaaaguguac

SEQ ID NO: 77 - RNA, 451 nt, Artificial Sequence
  CAA(Stem 1)-GUS mRNA
gcaacaacaacaacaacaacaacaacaacaacaacaacaacaagggcgccugccccaacaacaaccaugguccguccuguagaaaccccaacccgugaaaucaaaaaacucgacggccugugggcauucagucuggaucgcgaaaacuguggaauugaucagcguuggugggaaagcgcguuacaagaaagccgggcaauugcugugccaggcaguuuuaacgaucaguucgccgaugcagauauucguaauuaugcgggcaacgucugguaucagcgcgaagucuuuauaccgaaagguugggcaggccagcguaucgugcugcguuucgaugcggucacucauuacggcaaagugugggucaauaaucaggaagugauggagcaucagggcggcuauacgccauuugaagccgaugucacgccguauguuauugccgggaaaaguguac

SEQ ID NO: 78 - RNA, 457 nt, Artificial Sequence
  CAA(Stem 2)-GUS mRNA
gcaacaacaacaacaacaacaacaacaacaacaacaacaacaagggcugcgccugcagccccaacaacaaccaugguccguccuguagaaaccccaacccgugaaaucaaaaaacucgacggccugugggcauucagucuggaucgcgaaaacuguggaauugaucagcguuggugggaaagcgcguuacaagaaagccgggcaauugcugugccaggcaguuuuaacgaucaguucgccgaugcagauauucguaauuaugcgggcaacgucugguaucagcgcgaagucuuuauaccgaaagguugggcaggccagcguaucgugcugcguuucgaugcggucacucauuacggcaaagugugggucaauaaucaggaagugauggagcaucagggcggcuauacgccauuugaagccgaugucacgccguauguuauugccgggaaaaguguac

SEQ ID NO: 79 - RNA, 449 nt, Artificial Sequence
  (CAA)N-AUG-AUG-GUS mRNA
gcaacaacaacaacaacaacaacaacaacaacaacaacaacaacaaaccaugcaacaacaaaccaugguccguccuguagaaaccccaacccgugaaaucaaaaaacucgacggccugugggcauucagucuggaucgcgaaaacuguggaauugaucagcguuggugggaaagcgcguuacaagaaagccgggcaauugcugugccaggcaguuuuaacgaucaguucgccgaugcagauauucguaauuaugcgggcaacgucugguaucagcgcgaagucuuuauaccgaaagguugggcaggccagcguaucgugcugcguuucgaugcggucacucauuacggcaaagugugggucaauaaucaggaagugauggagcaucagggcggcuauacgccauuugaagccgaugucacgccguauguuauugccgggaaaaguguac

SEQ ID NO: 80 - RNA, 462 nt, Artificial Sequence
  CrPV IGR IRES
gggcgaauugggcccucuagaugcaugcucgagcggccgccagugugauggauaucuauguuggcugaugagguuuacgacuucuaaaaagcaaaaaugugaucuugcuuguaaauacaauuuugagagguuaauaaauuacaaguagugcuauuuuuguauuuagguuagcuauuuagcuuuacguuccaggaugccuaguggcagccccacaauauccaggaagcccucucugcgguuuuucagauuagguagucgaaaaaccuaagaaauuuaccugcuacauuucaagauaaacaagaaaauucacacauugaaaaugaagauaaaagacuuauguccgaacagaaagaaauuguacauuuuguuagcgaaggaauuaccccuaguacuacugcgcucccugauaucguuaaucuuucaacuaauuaucuggacaugacuacgagagaagauagaauu

SEQ ID NO: 81 - RNA, 1321 nt, Artificial Sequence
  CSFV .delta. 442.NS′ mRNA
gggagaccggaagcugucgacaagguuagcucuuucucguauacgauauuggauacacuaaauuucgauuuggucuagggcaccccuccagcgacggccgaaaugggcuagccaugcccauaguaggacuagcaaacggagggacuagccguaguggcgagcucccuggguggucuaaguccugaguacaggacagucgucaguaguucgacgugagcacuagcccaccucgagaugcuacguggacgagggcaugcccaagacacaccuuaacccuggcgggggucgcuagggugaaaucacauuaugugauggggguacgaccugauagggugcugcagaggcccacuagcaggcuaguauaaaaaucucugcuguacauggcacauggaguugaaucauuuugaauuauuauacaaaacaagcaaacaaaaaccagugggaguggaggaaccggguaccauggaucccagcuuucagguagauugcuuucuuuggcauguccgcaaacgaguugcagaccaagaacuaggugaugccccauuccuugaucggcuucgccgagaucagaaaucccuaagaggaaggggcagcacucuuggucuggacaucgagacagccacacgugcuggaaagcagauaguggagcggauucugaaagaagaauccgaugaggcacuuaaaaugaccauggccucuguaccugcgucgcguuaccuaaccgacaugacucuugaggaaaugucaagggaaugguccaugcucauacccaagcagaaaguggcaggcccucuuuguaucagaauggaccaggcgaucauggauaaaaacaucauacugaaagcgaacuucagugugauuuuugaccggcuggagacucuaauauugcuaagggcuuucaccgaagagggagcaauuguuggcgaaauuucaccauugccuucucuuccaggacauacugcugaggaugucaaaaaugcaguuggaguccucaucggaggacuugaauggaaugauaacacaguucgagucucugaaacucuacagagauucgcuuggagaagcaguaaugagaaugggagaccuccacucacuccaaaacagaaacgagaaauggcgggaacaauuaggucagaaguuuugaagaaauaagaugguugauugaagaagugagacacaaacugaagguaacagagaauaguuuugagcaaauaacauuuaugcaagccuuacaucuauugcuugaaguggagcaagagauaagaacuuucucauuucagcuuauuuaauaauaaaaaacacccuuguuucuacugaaauu

SEQ ID NO: 82 - RNA, 1197 nt, Artificial Sequence
  CSFV(128-442).NS′ mRNA
gggagaccggaagcugucgacuagccguaguggcgagcucccuggguggucuaaguccugaguacaggacagucgucaguaguucgacgugagcacuagcccaccucgagaugcuacguggacgagggcaugcccaagacacaccuuaacccuggcgggggucgcuagggugaaaucacauuaugugauggggguacgaccugauagggugcugcagaggcccacuagcaggcuaguauaaaaaucucugcuguacauggcacauggaguugaaucauuuugaauuauuauacaaaacaagcaaacaaaaaccagugggaguggaggaaccggguaccauggaucccagcuuucagguagauugcuuucuuuggcauguccgcaaacgaguugcagaccaagaacuaggugaugccccauuccuugaucggcuucgccgagaucagaaaucccuaagaggaaggggcagcacucuuggucuggacaucgagacagccacacgugcuggaaagcagauaguggagcggauucugaaagaagaauccgaugaggcacuuaaaaugaccauggccucuguaccugcgucgcguuaccuaaccgacaugacucuugaggaaaugucaagggaaugguccaugcucauacccaagcagaaaguggcaggcccucuuuguaucagaauggaccaggcgaucauggauaaaaacaucauacugaaagcgaacuucagugugauuuuugaccggcuggagacucuaauauugcuaagggcuuucaccgaagagggagcaauuguuggcgaaauuucaccauugccuucucuuccaggacauacugcugaggaugucaaaaaugcaguuggaguccucaucggaggacuugaauggaaugauaacacaguucgagucucugaaacucuacagagauucgcuuggagaagcaguaaugagaaugggagaccuccacucacuccaaaacagaaacgagaaauggcgggaacaauuaggucagaaguuuugaagaaauaagaugguugauugaagaagugagacacaaacugaagguaacagagaauaguuuugagcaaauaacauuuaugcaagccuuacaucuauugcuugaaguggagcaagagauaagaacuuucucauuucagcuuauuuaauaauaaaaaacacccuuguuucuacugaaauu

(19) United States
(12) Patent Application Publication
(10) Pub. No.: US 2010/0129861 A1
(43) Pub. Date: May 27, 2010

(54) Isolation and Use of Novel Mammalian DExH Box Helicases

(75) Inventors: Tatyana Vasllyevna Pestova, Brooklyn, NY (US); Christopher U.T. Hellen, Brooklyn, NY (US); Vera P. Pisareva, Brooklyn, NY (US); Andrey V. Pisarev, Brooklyn, NY (US)

Correspondence Address: HOFFMANN & BARON, LLP, 6900 JERICHO TURNPIKE, SYOSSET, NY, 11791, US

(73) Assignee: THE RESEARCH FOUNDATION OF STATE UNIVERSITY OF NEW YORK, ALBANY, NY (US)

(21) Appl. No.: 12/614,645

(22) Filed: Nov. 9, 2009

Related U.S. Application Data
(60) Provisional application No. 61/112,832, filed on Nov. 10, 2008.

(51) Int. Cl. C12P 21/00; C12N 9/48

(52) U.S. Cl. 435/68.1; 435/212

(57) ABSTRACT

The present invention relates to the isolation, purification, and use of novel mammalian DExH box helicases. In particular, the present invention relates to the isolation, purification and use of DHX29, a novel mammalian RNA helicase.

### CROSS-REFERENCE TO RELATED APPLICATIONS

This application claims priority to provisional application 61/112,832, filed Nov. 10, 2008, which is herein incorporated by reference in its entirety

### FUNDING STATEMENT

The invention was made in the course of research supported by NIH grants, under Sponsored Assigned Identification Numbers 5R01GM059660 and 5R01AI051340. As a result, the U.S. government may have certain rights in this invention.

### FIELD OF THE INVENTION

The present invention relates to the isolation, purification, and use of novel mammalian DExH box helicases. In particular, the present invention relates to the isolation, purification and use of DHX29, a novel mammalian NTPase and RNA helicase.

### BACKGROUND OF THE INVENTION

Eukaryotic protein synthesis begins with assembly of 48S initiation complexes at the initiation codon of mRNA, which typically requires at least 7 initiation factors (referred to as “eIFs”). These eIFs include eIFs 3, 2, 1, 1A, 4F, 4A and 4B, which cooperatively assist in formation of mRNAs.

Proteins, such as β-globin, serum albumin, myosin MYH6, and lysozyme, are encoded by mRNAs that have short, unstructured 5′ untranslated regions (5′-UTRs). These proteins are typically referred to as “house-keeping” proteins. In contrast, mRNAs that encode regulatory proteins such as proto-oncogenes, growth factors, their receptors, homeodomain proteins and transcription factors commonly have much longer 5′-UTRs that contain significant secondary structure. These proteins control many necessary processes, ranging from growth and development to innate immunity, cell cycle control, tumor invasion, and metastasis. Several translation initiation factors are over-expressed in tumors, which may cause cancer and/or affect its prognosis.

Current studies have focused on inhibitors of components of the eIF4F complex, and of pathways that signal to it as potential therapeutic targets for the treatment of cancers in mammals, particularly in humans. It had been determined that introduction of single GC-rich stems of increasing stability in a synthetic 5′ leader linked to a reporter open reading frame (ORF) progressively impaired translation of the reporter. However, it has now been discovered that the 7 eIFs are not sufficient for efficient 48S complex formation on mRNAs with highly structured 5′-UTRs that are translated in mammalian cells. Moreover, 48S complexes assembled in vitro on β-globin mRNA using these 7 eIFs and analyzed by primer extension inhibition (“toe-printing”) have revealed incorrect fixation of mRNA on the A-site side of the mRNA-binding channel. Sufficient and efficient formation of the 48S complex is desirable.

There is currently a need for a method of isolating and using a novel mammalian helicase in various applications, including in initiating translation on various mRNAs (including 48S complex formation), which serves an important role in normal and abnormal cellular and developmental processes. Further, there is a need to prepare and isolate a purified form of the novel mammalian helicase, which may be useful in a variety of therapeutic applications.

### SUMMARY OF THE INVENTION

In one embodiment, the invention provides a method of forming a 48S complex comprising the use of a DExH-box protein. The DExH-box protein may be any protein desired, and may be DHX29.

In another embodiment of the invention, there is provided a method of purifying a DExH-box protein comprising the steps of: performing a ribosomal salt wash; precipitating a first fraction of the ribosomal salt wash containing the DExH-box protein; applying the first fraction to a DEAE column to provide an eluted fraction; performing a step elution on a plurality of aliquots of the eluted fraction through a phosphocellulose column to provide a step-eluted fraction; subjecting a step-eluted fraction to a first liquid chromatography column to provide a first purified fraction; subjecting the first purified fraction to a second liquid chromatography column to provide a second purified fraction; and applying the second purified fraction to a hydroxyapatite column to elute a purified DExH-box protein.

In yet another embodiment of the invention there is provided a method of performing translation initiation involving ribosomal scanning comprising the use of one particular helicase: DHX29. Other embodiments of the invention include providing biochemical assays of DHX29's activities that permit a means of identifying and assaying inhibitors of DHX29 function, providing a method of using DHX29 to achieve therapeutic regulation of gene expression and providing a method of using DHX29 as a biomarker for diagnosis of human cancer.

### BRIEF DESCRIPTION OF THE DRAWINGS

FIG. 1 is one particular protocol for the purification of native DHX29.

FIG. 2 is a model of the domain organization of human DHX29.

FIG. 3 is an alignment of conserved motifs in the helicase core domains of human DHX29 and representative DExH-box proteins.

FIG. 4 is a characterization of purified native DHX29 (right lane) and protein molecular weight markers resolved by SDS-PAGE (left lane).

FIG. 5A is a toe-printing analysis of 48S ribosomal initiation complexes assembled on β-globin mRNA.

FIG. 5B is a toe-printing analysis of 48S ribosomal initiation complexes assembled on CAA-GUS and CAA (Stem)-GUS mRNAs.

FIG. 5C is a toe-printing analysis of 48S ribosomal initiation complexes assembled on neutrophil cytosolic factor 2 mRNA (NCF2).

FIG. 5D is a toe-printing analysis of 48S ribosomal initiation complexes assembled on Ser/Thr protein phosphatase CDC25 mRNA.

FIG. 5E is an analysis of formation of elongation complexes on CAA-Stem-3,4-MVHC-STOP mRNAs assayed by toeprinting (left panel) and by sucrose density gradient (“SDG”) centrifugation with subsequent monitoring of ³⁵S-MVHC tetrapeptide (right panel).

FIG. 6A is a toe-printing analysis of 48S complex assembly on β-globin mRNA.

FIG. 6B is a toe-printing analysis of 48S complex assembly on mRNA containing two AUG triplets.

FIG. 6C is a toe-printing analysis of 48S complex assembly on CAA-GUS Stem-1 mRNA.

FIG. 7A is a depiction of association of purified DHX29 with individual 40S and 60S subunits, 80S ribosomes, 40S/eIF3/(CUUU)₉ complexes and 43S complexes containing 40S subunits and eIFs 2/3/1/1A.

FIG. 7B is a depiction of association of purified DHX29 with yeast 40S subunits.

FIG. 7C is a depiction of association of purified DHX29 with 40S/eIF3/(CUUU)₉ complexes in the presence/absence of nucleotides as indicated (lanes 4-7).

FIG. 7D is a depiction of association of a DHX29 preparation containing a C-terminally truncated fragment resolved by SDS-PAGE (left panel) and its association with 40S subunits (right panel).

FIG. 8A is a thin-layer chromatography analysis of DHX29's NTPase activity in the presence/absence of SDG-purified 43S complexes containing 40S subunits and eIF2/3/1/1A.

FIG. 8B represents time courses of ATP hydrolysis by DHX29 in the presence/absence of (CUUU)₉ RNA, 18S rRNA, 43S complexes or 43S/(CUUU)₉.

FIG. 8C is a toe-printing analysis of 48S complexes assembled on CAA-GUS Stem-1 mRNA in the presence of SDG-purified 43S complexes, DHX29 and NTPs or non-hydrolyzable NTP analogues.

FIG. 9A is a representation of non-denaturing PAGE done to show unwinding of 13-bp RNA duplexes with 25 nt-long single-stranded overhanging 5′-regions by DHX29, 43S complexes, 43S/DHX29 complexes and eIF4A/eIF4F.

FIG. 9B is a representation of non-denaturing PAGE done to show unwinding of RNA duplexes corresponding to Stem-2, Stem-3 and Stem-4 with 25 nt-long single-stranded overhanging 5′-regions by DHX29, 43S complexes, 43S/DHX29 complexes and eIF4A/eIF4F.

FIG. 10A is representation of SDG-purified 43S complexes containing different amounts of DHX29 and analyzed by SDS-PAGE and fluorescent SYPRO staining.

FIG. 10B is a toe-printing analysis of 48S complex formation on CAA-GUS Stem-1 mRNA in the presence of SDG-purified free 43S complexes and different amounts of DHX29.

FIG. 10C is a toe-printing analysis of 48S complex formation on CAA-GUS Stem-1 mRNA in the presence of DHX29-free 43S complexes, DHX29-saturated 43S complexes or DHX29-saturated 43S complexes and either DHX29-free 43S complexes or 43S/eIF3/(CUUU)₉ complexes.

FIG. 11A is a toe-printing analysis of 40S/IRES binary complexes assembled on the CrPV IGR IRES.

FIG. 11B is a toe-printing analysis of 40S/IRES binary complexes assembled on the CrPV IGR IRES.

FIG. 11C is a toe-printing analysis of wt and Δdomain II CSFV IRESs.

### DETAILED DESCRIPTION OF THE PREFERRED EMBODIMENTS

The present invention relates to the isolation, purification, and use of novel mammalian helicases, and in particular of DExH-box helicases (including DEAH-box helicases). In one embodiment, the invention relates to the isolation, purification, and use of one particular helicase, DHX29 [Seq. ID No. 1]. It has been determined that DHX29 is a novel putative initiation factor. It has been discovered that DHX29 may bind 40S subunits, which include the amino acid sequences forming ribosomal protein rpSA [Seq. ID No. 2], ribosomal protein rpS2 [Seq. ID No. 3], ribosomal protein rpS3 [Seq. ID No. 4], ribosomal protein rpS3a [Seq. ID No. 5], ribosomal protein rpS4X [Seq. ID No. 6], ribosomal protein rpS5 [Seq. ID No. 7], ribosomal protein rpS6 [Seq. ID No. 8], ribosomal protein rpS7 [Seq. ID No. 9], ribosomal protein rpS8 [Seq. ID No. 10], ribosomal protein rpS9 [Seq. ID No. 11], ribosomal protein rpS10 [Seq. ID No. 12], ribosomal protein rpS11 [Seq. ID No. 13], ribosomal protein rpS12 [Seq. ID No. 14], ribosomal protein rpS13 [Seq. ID No. 15], ribosomal protein rpS14 [Seq. ID No. 16], ribosomal protein rpS15 [Seq. ID No. 17], ribosomal protein rpS15A [Seq. ID No. 18], ribosomal protein rpS16 [Seq. ID No. 19], ribosomal protein rpS17 [Seq. ID No. 20], ribosomal protein rpS18 [Seq. ID No. 21], ribosomal protein rpS19 [Seq. ID No. 22], ribosomal protein rpS20 [Seq. ID No. 23], ribosomal protein rpS21 [Seq. ID No. 24], ribosomal protein rpS23 [Seq. ID No. 25], ribosomal protein rpS24 [Seq. ID No. 26], ribosomal protein rpS25 [Seq. ID No. 27], ribosomal protein rpS26 [Seq. ID No. 28], ribosomal protein rpS27 [Seq. ID No. 29], ribosomal protein rpS27A [Seq. ID No. 30], ribosomal protein rpS28 [Seq. ID No. 31], ribosomal protein rpS29 [Seq. ID No. 32], ribosomal protein rpS30 [Seq. ID No. 33], and the DNA sequence forming H. sapiens 18S [Seq. ID No. 34]. Further, it has been discovered that DHX29 may hydrolyze ATP, GTP, UTP and CTP. Further, NTP hydrolysis by DHX29 has been found to be strongly stimulated by 43S complexes, and is required for DHX29's activity in promoting formation of the 48S complex.

Although the studies described herein relate to DHX29, it will be understood that the uses and isolation/purification methods described herein relate generally to DExH-box helicases generally, and are not limited to DHX29. DHX29 may be used in various functions, including aiding in forming a 48S initiation complex, following binding of a 43S preinitiation complex to the 5′-proximal region of a mRNA, in aiding in ribosomal scanning, and in ensuring fixation of mRNA in the ribosomal mRNA-binding cleft. As will be described in more detail below, DHX29 may be used alone or in combination with other complexes and/or eukaryotic initiation factors (eIFs).

As discussed herein, DExH-box proteins have been discovered to be useful in various therapeutic and beneficial applications. Although the DExH-box protein in any form may be useful, it is especially preferred to utilize the DExH-box protein in its purified form, to provide the most desirable and reproducible results. As used herein, the term “purified” refers to the protein in an apparently homogenous form, that is, at least about 95% pure, and more desirably at least about 98% pure.

Purification and/or Isolation of DExH-Box Proteins

The invention includes a protocol for isolating and purifying DExH-box proteins (such as DHX29) from mammalian cells. The protocol set forth herein may be used to isolate and purify DExH-box proteins from any mammalian cells, including human HeLa cells and rabbit reticulocytes. It will be understood that rabbit factors/ribosomal subunits may be interchangeably used for human equivalents, since the sequences are similar. Thus, the present invention may be directed to human initiation factors and their rabbit equivalents. The protocol set forth herein provides purified DExH-box proteins to a level of near-homogeneity, i.e., at least about 98% pure.

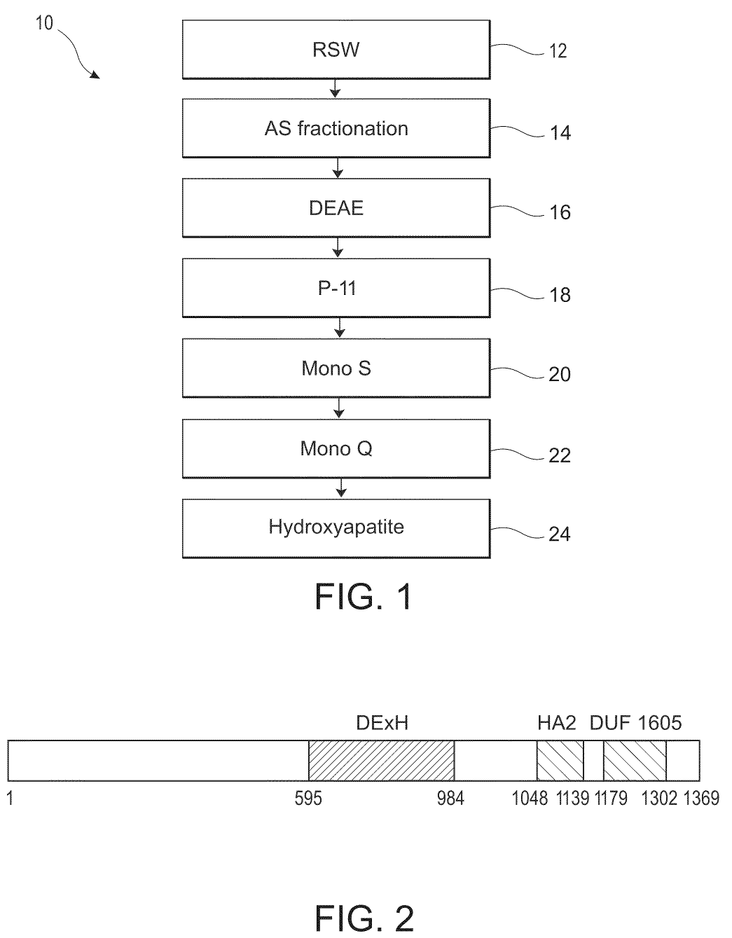

In one embodiment, the invention relates to a method of isolating and/or purifying a DExH-box protein, including DHX29. One preferred method 10 of purifying and isolating DHX29 is depicted in FIG. 1. In a first step 12, a ribosomal salt wash may be prepared. Any desired ribosomal salt wash may be used, and in a preferred embodiment, the ribosomal salt wash may be prepared as described in Pisarev et al., Methods Enzymol., 430: 147-177 (2007), the contents of which are incorporated herein by reference. In this preparation, a polysomal suspension derived from a mammalian reticulocyte lysate may be stirred in a cooled environment, such as on ice. Although a ribosomal salt wash is preferred, it is contemplated that other starting materials may be used to purify DExH-box proteins involved in splicing, chromatin remodeling and other nuclear functions.

Thereafter, a desired amount of salt, preferably about 4 M, is added, preferably in a drop-wise manner. It may be desired to add a quantity of salt while continuously stirring the mixture. Any salt may be used, and in one embodiment, the salt is potassium chloride. The mixture is stirred continuously until there is approximately a 0.5 M salt final concentration. After further stirring, the suspension may be centrifuged. Desirably, the centrifuging is conducted in a Beckman Ti 50.2 rotor at approximately 45,000 rpm, for about 4.5 hours at 4° C., but any centrifugation technique desired may be used. The supernatant will be the ribosomal salt wash (“RSW”).

In a next step 14, the fraction containing the DExH-box protein (such as DHX29) is then precipitated from the RSW, desirably with ammonium sulfate. DHX29 has been found to be in the 0-40% ammonium sulfate fraction, which may be prepared by adding ammonium sulfate (preferably in a powdered state) to the RSW while stirring the RSW. Desirably, the RSW is kept in a chilled state, and may be maintained on ice. Any amount of ammonium sulfate may be used, and desirably is added in an amount of about 240 g/L RSW. The resulting suspension may then be centrifuged. Centrifugation may be performed by any desired means, and preferably is conducted in a Sorvall SS34 rotor at approximately 15,000 rpm for about 20 minutes at about 4° C. The resulting product may then be removed from the apparatus. In one form, the resulting product is in pellet form, but may be in any resulting shape or state. The resulting product may then be dissolved in a 5-7 ml buffer (“buffer A”) with about 100 mM KCl. Preferably, the dissolved resulting product is dialyzed against 1 L of the buffer A overnight in a chilled state (at about 4° C.), and clarified by centrifugation at about 10,000 rpm for about 10 min at about 4° C. Any desired buffer A may be used, and desirably, the buffer A includes about 20 mM tris-HCl, having a pH of about 7.5, 2 mM DTT, 0.1 mM EDTA, and about 10% glycerol.

In a next step 16, the dialyzed 0-40% ammonium sulfate fraction is then applied to a DEAE (DE52) column, equilibrated with buffer A and 100 mM salt. Preferably, the salt is KCl, but any desired salt may be used. The fraction containing DHX29 is then eluted in the flow-through fraction with buffer A and 100 mM salt.

In one embodiment, in a next step 18, a plurality of aliquots, each of from 15-20 ml, of the resulting solution may then be applied to a phosphocellulose (P11) column. The aliquots are desirably equilibrated with buffer A and 100 mM salt. Step elution is then performed. In one embodiment, the step elution process may begin with buffer A and about 200 mM salt, followed by buffer A and about 300 mM salt; buffer A and about 400 mM salt; buffer A and about 500 mM salt; and buffer A and about 1000 mM salt. Any desired step elution may be performed, generally with increasing amounts of salt. Further, any number of aliquots may be used, preferably between 4-6 aliquots being used.

In a next step 20, one fraction is selected and is then dialyzed overnight. In a desired embodiment, the fraction including buffer A and about 400 mM salt is dialyzed overnight, but any fraction may be used if desired. The dialyzation is preferably performed in a cooled environment, such as at about 4° C., against about 1 liter of a second buffer (“buffer B”) and 100 mM salt. Any desired salt may be used, and preferably the salt for this step 20 is the same as the salt used in previous steps. Buffer B may be the same or may be different than buffer A, and include any desired buffering material. Most desirably, buffer B differs from buffer A, and includes about 20 mM HEPES, with a pH of 7.5, 0.1 mM EDTA, 2 mM DTT, and 5% glycerol. After the overnight dialyzation, the fraction may then be loaded onto a liquid chromatography column. Preferably, the fraction is loaded onto a FPLC monoS HR 5/5 column that has previously been pre-equilibrated with buffer B and 100 mM salt. The target proteins to be purified may then be eluted with a mixture of buffer B and about 100-500 mM salt gradient. For embodiments where the target protein to be purified is DHX29, the preferable elution of DHX29 is at about 300 mM KCl (corresponding to fraction 28).

In the next step 22, the eluted fraction from step 20 is then dialyzed overnight at about 4° C. It may be desired to dialyze the eluted fraction along with the neighboring fractions (generally corresponding to fractions 27 and 29). The fraction(s) are dialyzed against 1 liter of a third buffer (“buffer C”) and 100 mM salt. Buffer C may be the same or may be different from buffer A and/or buffer B, and may include any buffering mixture desired. Preferably, buffer C is different and includes about 20 mM Tris-HCl, with a pH of 7.5, 0.1 mM EDTA, 2 mM DTT, and 5% glycerol. After dialysis, the fraction may then be diluted with buffer C to 30 mM salt and loaded onto a liquid chromatography column. Desirably, the fraction is loaded onto a FPLC MonoQ HR 5/5 column, which has been pre-equilibrated with buffer C and 30 mM salt. The target protein may then be eluted with a mixture of buffer C and about 30-500 mM salt gradient. For embodiments where the target protein to be purified is DHX29, the preferable elution of DHX29 is at about 250 mM salt (which generally corresponds to fraction 27).

Finally, in the last step 24, the eluted fraction of target protein is then dialyzed overnight in a cooled environment (such as at about 4° C.). It may be desired to dialyze the neighboring eluted fractions concurrently (generally corresponding to fractions 26 and 28). The eluted fraction(s) may be dialyzed against 1 liter of another buffer (“buffer D”). Buffer D may be the same or may be different than buffer A, buffer B, and/or buffer C, and may include any desired buffering mixture. Desirably, buffer D includes a mixture of about 20 mM Tris-HCl, with a pH of 7.5, 5% glycerol, and 100 mM salt. The dialyzed fraction may then be diluted approximately five-fold, with a 20 mM phosphate buffer. Any phosphate buffer may be used if desired, and desirably the phosphate buffer is a mixture of KH₂PO₄ and K₂HPO₄, adjusting the pH to about 7.5 and adding about 5% glycerol. The sample may then be applied to a hydroxyapatite column, which is preferably pre-equilibrated with the phosphate buffer. The target proteins are then eluted with a 20-500 mM phosphate buffer gradient. For embodiments where the target protein to be purified is DHX29, the preferable elution of DHX29 is at about a 300 mM phosphate buffer (which generally corresponds to fraction 36).

The eluted product is then a substantially fully purified protein, and desirably is a substantially fully purified DExH-box protein, such as DHX29.

The process 10 set forth above provides one method of isolating and purifying target proteins, such as DExH-box proteins, including DHX29. Any process for purification and isolation of DHX29 may be incorporated if desired. For example, any or all of the above purification steps may be used if desired. For example, a substantially purified protein may be prepared without the last step 24 of exposure to a hydroxyapatite column. In other embodiments, one or more of steps 12, 14, 16, 18, 20, 22, or 24 may be omitted if desired. It will be understood that, for optimal purification, each step should be performed, but any may be omitted if desired. In other embodiments, DHX29 can be over-expressed in E. coli, yeast, insect cells or mammalian cells in recombinant form, with or without N-terminal or C-terminal affinity tags, which may be but are not limited to His6 (hexahistidine tag); GST (glutathione S-transferase); MBP (maltose-binding protein); FLAG (FLAG-tag peptide); BAP (biotin acceptor peptide); STREP (streptavidin-binding peptide); or CBP (calmodulin-binding peptide). Purification of recombinant DHX29 may include one or more of steps 12, 14, 16, 18, 20, 22, and 24, and will preferably include each of the listed steps. Further, purification of recombinant DHX29 may include the use of one or more appropriate affinity matrix, particularly if the recombinant DHX29 has N-terminal and/or C-terminal affinity tags. Use of such affinity matrix may aid in reducing the number of additional downstream steps to be used to fully purify the protein.

In addition, alternative steps may be performed if desired. For example, the process may include a gel-filtration step performed at any desired point in the process. Further, any desired ion-exchange columns and matrices may be used in place of or in combination with the Mono Q and Mono S columns described herein. Finally, alternative or additional buffer solutions may be used in the purification process outlined above. For example, buffer A may include mM tris in an amount of from 10-30 mM, having a pH of from about 6-9, having about 1-3 mM DTT, about 0.01-0.5 mM EDTA, and about 5-20% glycerol. As explained above, buffer B preferably differs from buffer A, and in one embodiment includes about 10-30 mM HEPES, with a pH of from 6-9, about 0.01-0.5 mM EDTA, 1-3 mM DTT, and 1-10% glycerol. Buffer C may be the same or may be different from any other buffer used, and may include about 10-30 mM Tris-HCl, with a pH of from 6-9, about 0.01-0.5 mM EDTA, 1-3 mM DTT, and 1-10% glycerol. Finally, buffer D may likewise be the same or may be different from any of the buffers used herein, and may include a mixture of about 10-30 mM Tris-HCl, with a pH of 6-9, 1-10% glycerol, and 50-150 mM salt. As will be understood, any of the buffers (A-D) may include any combination of the above components as desired.

The process described herein is not intended to be limited to the particular concentrations and compositions, and it is understood that equivalent columns, solutions, and equipment may be used if desired.

### Structure of DHX29

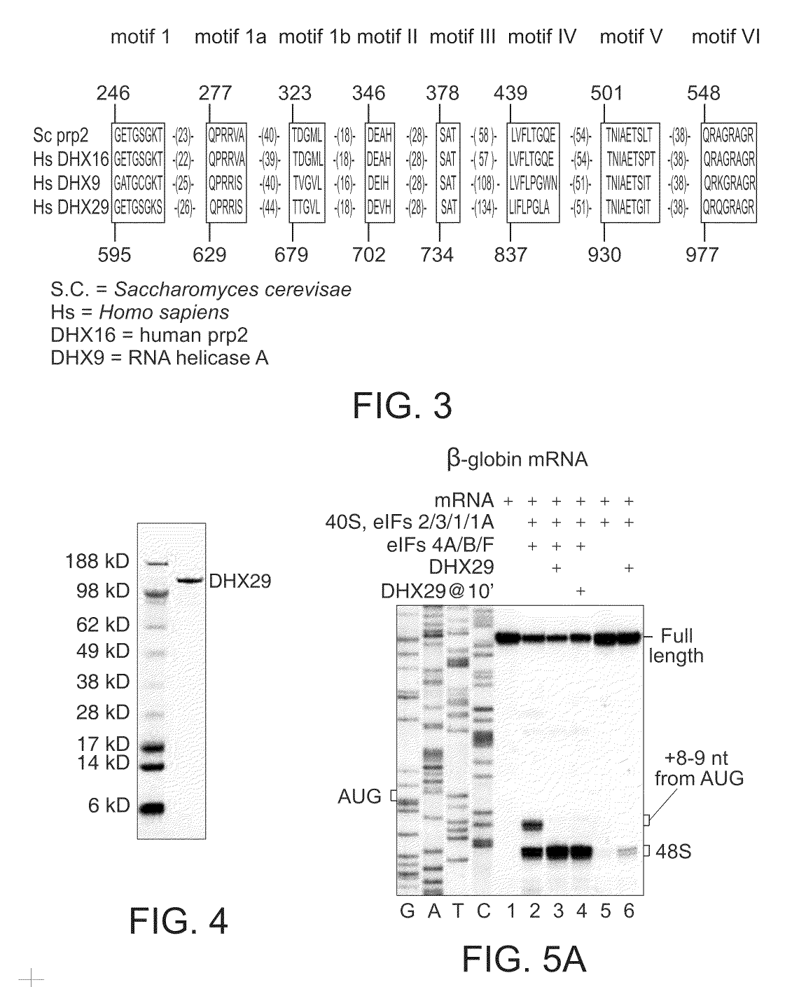

DHX29 is a DExH-box mammalian RNA helicase. DHX29 is a 1369 amino-acid long, 155 kDa protein (Genbank accession NP_{—}061903) [Seq. ID No. 1]. DHX29 belongs to the DEx/HD box family of helicases, and particularly the DEAH subfamily of helicases. As depicted in FIG. 2, DHX29 contains a helicase domain 32, a helicase associated domain of unknown function 34, and an associated DUF1605 domain of unknown function 36. The helicase domain (referenced as DExH) contains all of the consensus sequence motifs that are characteristic of DEAH helicases. DHX29 has C-terminally located helicase associated HA2 domain. A model of the conserved motifs of the helicase core domain of human DHX29 is set forth in FIG. 3.

The characterization of purified native DHX29 is depicted in FIG. 4. Various biochemical properties of DHX29 allow it to play various important roles in the initiation of translation (i.e., protein synthesis) in higher eukaryotes. In particular, DHX29 has ATPase, GTPase, CTPase and UTPase activities. In particular, the NTPase activity of DHX29 is weakly stimulated by random RNA, but is strongly stimulated by ribosomal 40S subunits, as set forth above, and by 18S ribosomal RNA. It has been determined that fully purified DHX29, such as that prepared by the process 10 described above, does not have processive helicase activity in the presence of any NTP, and further fully purified DHX29 binds to ribosomal 40S subunits in the absence of other translational components. Fully purified DHX29 is a stable constituent of ribosomal 43S complexes.

Table 1 below identifies DHX29 by LC/nanospray tandem mass-spectrometry of tryptic peptides. The amino acid residues are numbered according to the sequence of H. sapiens DHX29.

TABLE 1
Identification of DHX29

| Deduced Sequence | Amino Acid Residues |
|---|---|
| SLEEEEKFDPNER | 251-263 [Seq. ID No. 35] |
| SPNPSFEK | 394-401 [Seq. ID No. 36] |
| DLFIAK | 489-494 [Seq. ID No. 37] |
| VVVVAGETGSGK | 590-601 [Seq. ID No. 38] |
| ASQTLSFQEIALLK | 1204-1217 [Seq. ID No. 39] |
| LACIVETAQGK | 1243-1253 [Seq. ID No. 40] |
| VLIDSVLR | 1334-1341 [Seq. ID No. 41] |
| ILQIITELIK | 1356-1365 [Seq. ID No. 42] |

Table 2 below identifies the composition of ΔDHX29 by LC/nanospray tandem mass-spectrometry of tryptic peptides. The amino acid residues are numbered according to the sequence of H. sapiens DHX29 [Seq. ID No. 1].

TABLE 2
Identification of ΔDHX29

| Deduced Sequence | Amino Acid Residues |
|---|---|
| IIGVINEHK | 98-106 [Seq. ID No. 43] |
| SLEEEEKFDPNER | 251-263 [Seq. ID No. 44] |
| VVVVAGETGSGK | 590-601 [Seq. ID No. 45] |
| VCDELGCENGPGGR | 642-655 [Seq. ID No. 46] |
| NSLCGYQIR | 656-664 [Seq. ID No. 47] |

### Methods of Using DHX29

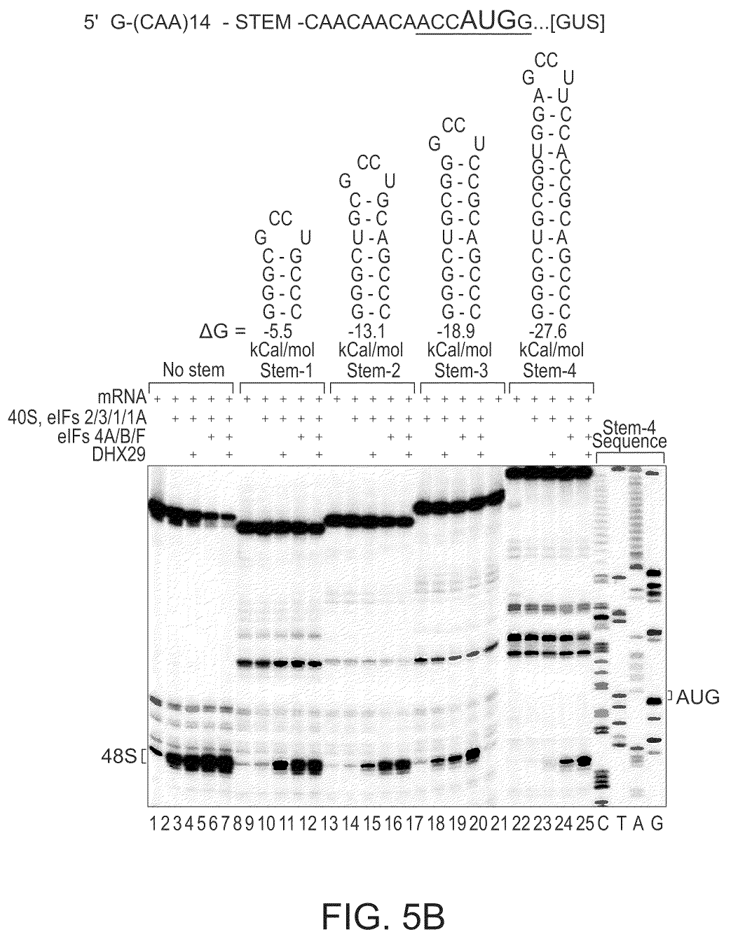

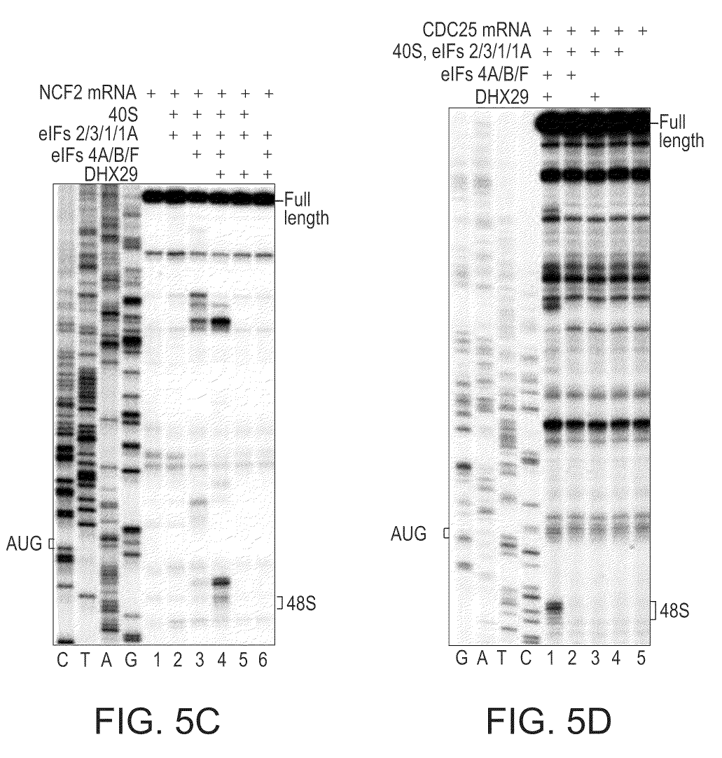

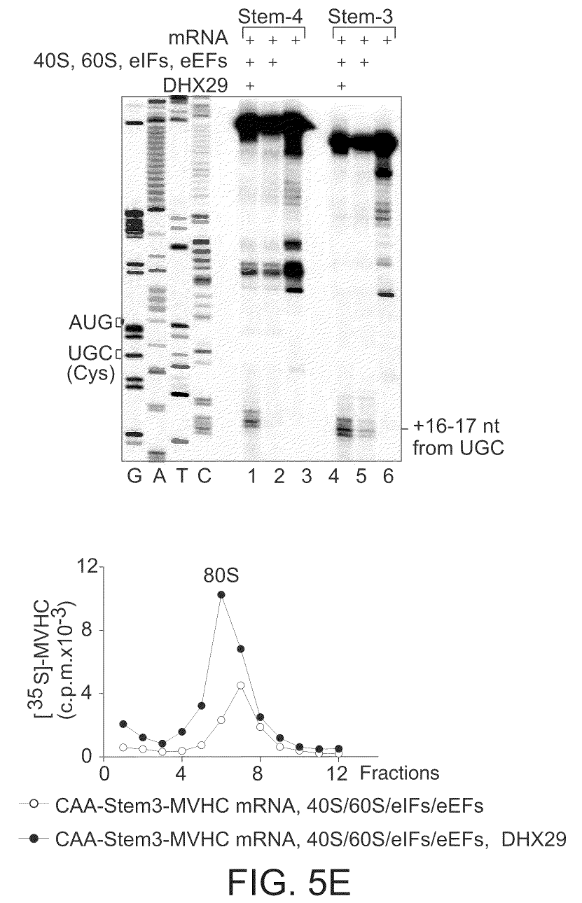

As will be described in more detail below, fully purified DHX29, such as that prepared by the purification process 10 described above, may promote proper fixation of mRNA in the mRNA-binding cleft of the ribosomal 40S subunit in 48S initiation complexes assembled at the initiation codon. Such proper fixation may be apparent in toe-printing analyses of 48S complexes assembled on native capped β-globin mRNA [Seq. ID No. 48] as suppression of aberrant toe-prints at positions +8-9 nt relative to the AUG initiation codon (A=+1) and enhancement of correct toe-prints at positions +15-17 nt that correspond to the leading edge of the 40S subunit. A toe-printing analysis of 48S ribosomal initiation complexes assembled on β-globin mRNA is depicted in FIG. 5A.

Further, DHX29 further enhances the formation of 48S initiation complexes by several means. First, DHX29 may be used to enhance the process of ribosomal scanning, functioning synergistically with eIF4A [Seq. ID No. 49]/eIF4B [Seq. ID No. 50]/eIF4F (which is a heterotrimer comprising eIF4A [Seq. ID No. 49], eIF4E [Seq. ID No. 51] and eIF4G [Seq. ID No. 52]) to enhance scanning on synthetic and/or natural mRNAs with highly structured 5′-UTRs. In addition, DHX29 may be used to functionally replace any or all of eIF4A/eIF4B/eIF4F to promote scanning on mRNAs with 5′-UTRs with weak or no significant secondary structure in their 5′-UTRs. Such use may be important as scanning may not effectively occur without DHX29 on mRNAs with the most highly structured 5′-UTRs. In addition, DHX29 ensures correct fixation of mRNA in the ribosomal mRNA-binding channel of 48S complexes after scanning, following arrest at the initiation codon, thus increasing the proportion of correctly assembled 48S complexes. These and other features will be more adequately and thoroughly described in the Examples set forth below.

As will be described in more detail in the Examples, in conjunction with other defined components of the translation apparatus (including but not limited to 40S ribosomal subunits, initiator tRNA [Seq. ID No. 53], GTP, ATP and eukaryotic initiation factors such as eIF1 [Seq. ID No. 54], eIF1A [Seq. ID No. 55], eIF2 (which is comprised of three subunits: subunit 1 [Seq. ID No. 56], subunit 2 [Seq. ID No. 57], subunit 3 [Seq. ID No. 58]), eIF3 (which is comprised of thirteen subunits: eIF3A [Seq. ID No. 59], eIF3B [Seq. ID No. 60], eIF3C [Seq. ID No. 61], eIF3D [Seq. ID No. 62], eIF3E [Seq. ID No. 63], eIF3F [Seq. ID No. 64], eIF3G [Seq. ID No. 65], eIF3H [Seq. ID No. 66], eIF3I [Seq. ID No. 67], eIF3J [Seq. ID No. 68], eIF3K [Seq. ID No. 69], eIF3L [Seq. ID No. 70], eIF3M [Seq. ID No. 71]), eIF4A [Seq. ID No. 49], eIF4B [Seq. ID No. 50], eIF4E [Seq. ID No. 51], and eIF4G [Seq. ID No. 52]), fully purified DHX29 may enable ribosomal 43S preinitiation complexes assembled with the above components to scan synthetic mRNA 5′-UTRs that contain modest secondary structure. Toe-printing analysis of 48S ribosomal initiation complexes assembled on CAA-GUS mRNAs containing stems of various stabilities is depicted in FIG. 5B. Further, fully purified DHX29 may enable ribosomal 43S preinitiation complexes assembled with the above components to scan the wild-type 5′-UTRs of natural mRNAs that contain extensive secondary structure, such as neutrophil cytosolic factor 2 mRNA (as shown in FIG. 5C) [Seq. ID No. 72] or CDC25 mRNA (as shown in FIG. 5D) [Seq. ID No. 73].

DHX29 may be used to aid in various processes and mechanisms. In one embodiment, DHX29 may be used in the synthesis of eukaryotic proteins. Eukaryotic protein synthesis typically begins with the assembly of 48S initiation complexes at the initiation codon of mRNA, which typically requires at least seven initiation factors (eIFs). First, various initiation factors bind to the 40S subunit to form a 43S preinitiation complex. eIF4F, eIF4A and eIF4B cooperatively unwind the cap-proximal region of mRNA allowing attachment of the 43S complexes. The 43S preinitiation complex (comprising a 40S ribosomal subunit, initiator tRNA, eIF2, eIF3, eIF1 and eIF1A) then attaches to the 5′-proximal region of the unwound mRNA. Attachment of a 43S complex is typically mediated by eIF4F (which, as set forth above, comprises eIF4E, eIF4A and eIF4G), eIF4A and eIF4B. In addition, eIF4F, eIF4A and eIF4B also assist 43S complexes during scanning.

In typical processes, the ribosomal subunits then scan along the 5′-UTR to the initiation codon where they stop, forming 48S complexes with established P-site codon-anticodon base pairing. It will be understood that “scanning” refers to unwinding of secondary structure in the 5′ leader, 5′-3′ movement of the 43S complex, and monitoring of interactions between the tRNA^{Met}ᵢ [Seq. ID No. 53] anticodon and triplets in the leader to prevent codon-anticodon mismatches and to signal establishment of correct base-pairing so that eIF2 hydrolyzes its bound GTP and loses affinity for Met-tRNA^{Met}ᵢ.

However, eIF2, eIF3, eIF1, eIF1A, eIF4A, eIF4B and eIF4F (collectively referred to as eIFs) have been found to be insufficient alone for an efficient and practical 48S complex formation on mRNAs with long structured 5′-UTRs. In particular, eIFs do not generally support high-level formation of 48S complexes on mRNAs containing longer and more stable stems, such as CAA-GUS stem-3 [Seq. ID No. 74] and stem-4 [Seq. ID No. 75] mRNAs (FIG. 5B). In addition, eIFs support only very weak 48S complex assembly on cellular neutrophil cytosolic factor 2 (NCF2) mRNA [Seq. ID No. 72] containing a 168 nt-long 5′-UTR. Finally, eIFs have been found not to be sufficient to promote 48S complex formation at all on Ser/Thr protein phosphatase CDC25 mRNA [Seq. ID No. 73], which contains a 271 nt-long 5′-UTR.

Due to the lack of sufficiency of eIFs on long, highly-structured mRNAs, DExH-box proteins, in particular DHX29, may be used in this process to more efficiently form a 48S complex on mRNAs with long structured 5′-UTRs. Inclusion of DHX29 in an in vitro reconstituted system has been found to strongly increase 48S complex formation on such mRNAs. Specifically, DHX29 may be used to bind 40S subunits and provide a stable constituent of 43S complexes. Further, DHX29 aids in forming the 48S complex by efficiently hydrolyzing ATP, GTP, UTP and CTP. Further, NTP hydrolysis by DHX29 is strongly stimulated by 43S complexes. In this fashion, DHX29 may be used to greatly aid in the formation of 48S complexes. Use of DHX29 may be used to increase 48S complex formation by a significant amount. In some embodiments, the use of DHX29 may increase 48S formation by any amount from at least 2-fold up to about 40-fold. In a preferred embodiment, use of DHX29 increases 48S complex formation from about 3-fold to about 20-fold. DHX29 may be controlled to increase 48S formation to any amount desired, including at least 3-fold, at least 5-fold, at least 10-fold, at least 20-fold and at least 40-fold.

In one embodiment, DHX29 may be incorporated into the 48S complex forming process in addition to the traditional eIFs described above. Alternatively, DHX29 may be included in 48S complex formation in the absence of any or all of the eIFs, including but not limited to eIF4A, eIF4B and eIF4F. DHX29 and at least eIF4F, with or without eIF4B synergistically promote efficient 48S complex formation on mRNAs with structured and stable 5′-UTRs. In some processes, such as those including NCF2 or CDC25 mRNAs, DHX29 and at least eIF4F, with or without eIF4B, and may be used in conjunction to promote 48S complex formation. In some embodiments, DHX29 may be used to assist with ribosomal scanning, and not used during initial attachment of 43S complexes. In some embodiments, DHX29 may be used for efficient 48S complex formation on mRNAs with highly structured 5′-UTRs and also to suppress the aberrant +8-9 nt toe-print.

In other uses, DHX29 may be bound to ribosomal complexes so as to include conformational changes near the mRNA-binding cleft that accommodate the 3′-portion of mRNA. In some embodiments, DHX29 may be used to increase leaky scanning, thus enhancing 48S complex formation on the second AUG codon of mRNA containing two AUG triplets, irrespective of the presence of eIF1 or eIF1A.

The DExH-box protein DHX29 may be used as a factor that is required for efficient initiation on mRNAs with long structured 5′-UTRs, which typically encode regulatory proteins. DHX29 may additionally be used to modify altered ribosomal conformations to enhance the processivity of scanning complexes. Further, DHX29 may be used to stabilize binding of mRNA in the mRNA-binding channel of the 40S subunit near its entrance. Finally, DHX29 may be used to remodel ribonucleo-protein complexes without extensive unwinding of RNA duplexes.

In some embodiments, analysis of DHX29 levels may be used in diagnosing diseases, including but not limited to cancer. Further, inhibition of DHX29 itself may be used in treating such diseases. The requirement for initiation factors between different mRNAs is non-uniform. Further, translation of some mRNAs is dependent on the activity of factors that promote ribosomal attachment to and scanning on mRNA. Consequently, translation of some mRNAs may be selectively and disproportionately affected by inhibition of the activity of these factors or down-regulation of their expression levels. Prior research has established that mRNAs that encode proteins that are involved in different aspects of malignancy are particularly dependent on eIF4F. As such, agents that block the activities of eIF4F and its components may thus be used as potential therapeutic agents for such malignancy.

Translation of mRNAs with long, structured 5′UTRs (which includes mRNAs encoding proteins that promote cell growth, cell cycle progression, inhibition of cell death and tumor growth and innate immune responses) is dependent on DHX29. In contrast, translation of ‘house-keeping’ mRNAs is not dependent on DHX29. Further, translation of CDC25, a regulator of the cell cycle, has been found to be dependent on DHX29. Consequently, DHX29 may thus be used as a target for therapeutic intervention.

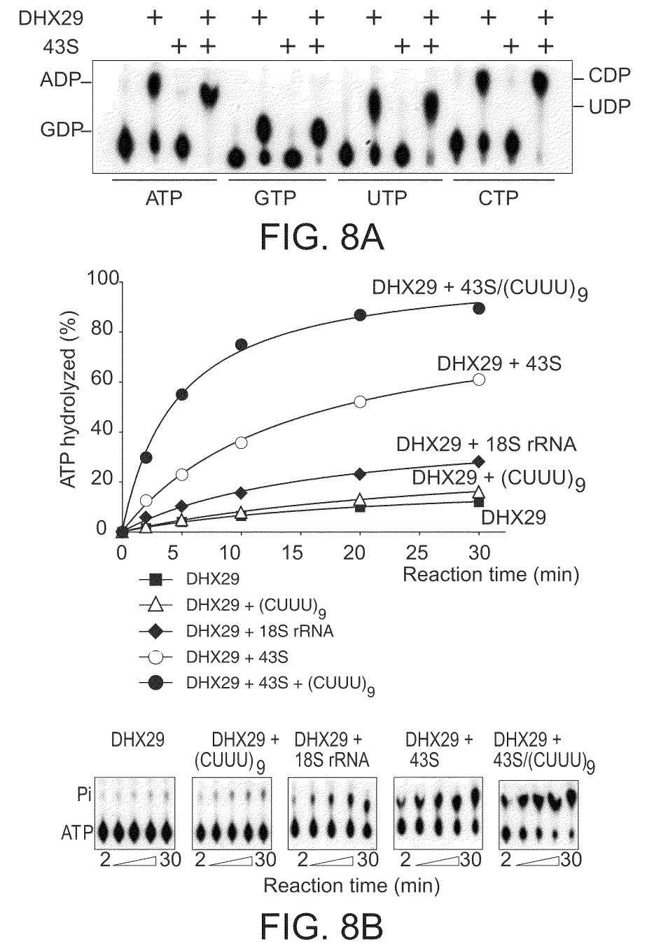

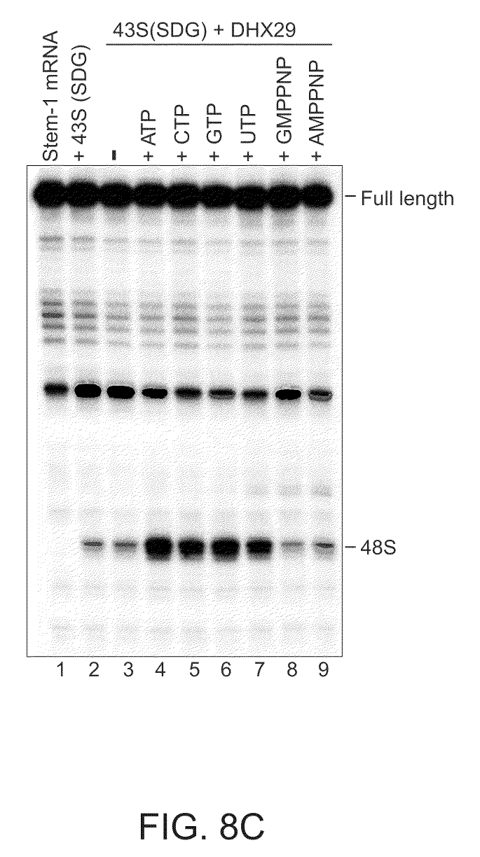

In one embodiment of the present invention, inhibitors of DHX29's biochemical activities (such as nucleotide binding and hydrolysis, binding to the ribosomal 40S subunit, promoting ribosomal scanning and correct assembly of 48S complexes on mRNA) may be used as therapeutic agents in the treatment of cancer. Assays of DHX29's biochemical activities that are required for its ability to mediate translation of mRNAs with long and highly structured 5′UTRs may be used to identify potential specific inhibitors of DHX29. Further, such assays may be used to test their inhibition of DHX29's activity in translation initiation. Through experimentation described in the Examples below, for example, it has been found that GMP-PNP and AMP-PNP, inhibitors of DHX29's NTPase activity, specifically block its function in translation initiation (FIG. 8C, compare lanes 4, 6 with 8, 9).

Thus, DHX29 may be used as a biomarker for cancerous tissues. As with many eIFs, the number of molecules of DHX29 in cells is lower than the number of ribosomes. Thus, just as a reduction in levels of active DHX29 by inhibitors would inhibit translation of proteins that promote malignancy, enhanced levels of DHX29 may promote expression of such proteins. Moreover, DHX29 may be upregulated in malignant melanomas, lymphomas, ovarian endometroid carcinoma and ovarian serous adenocarcinoma.

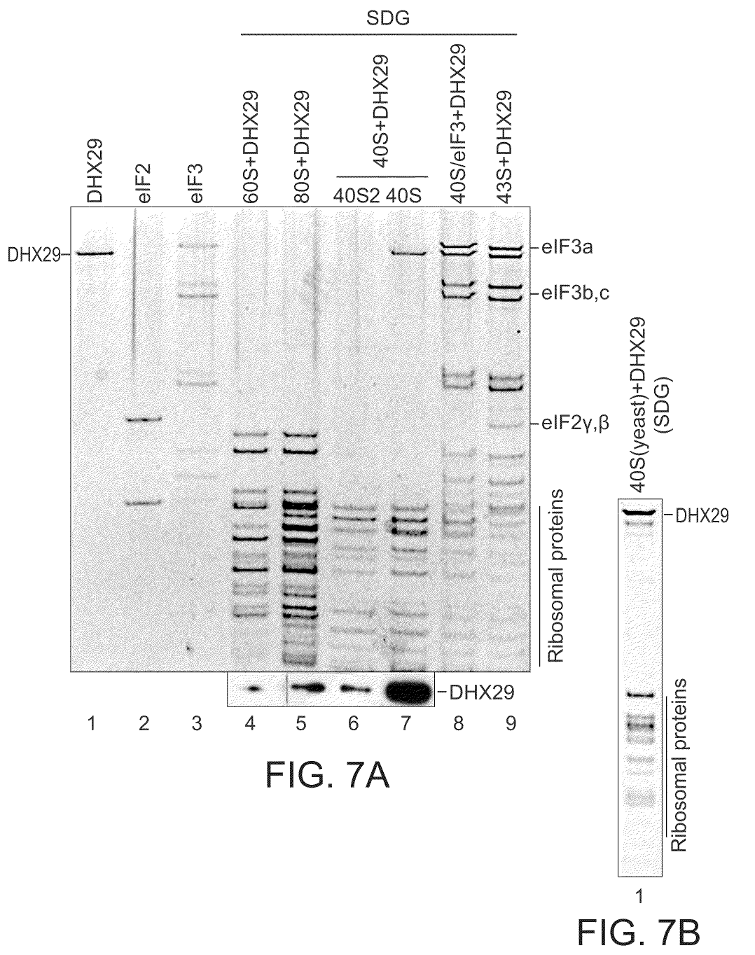

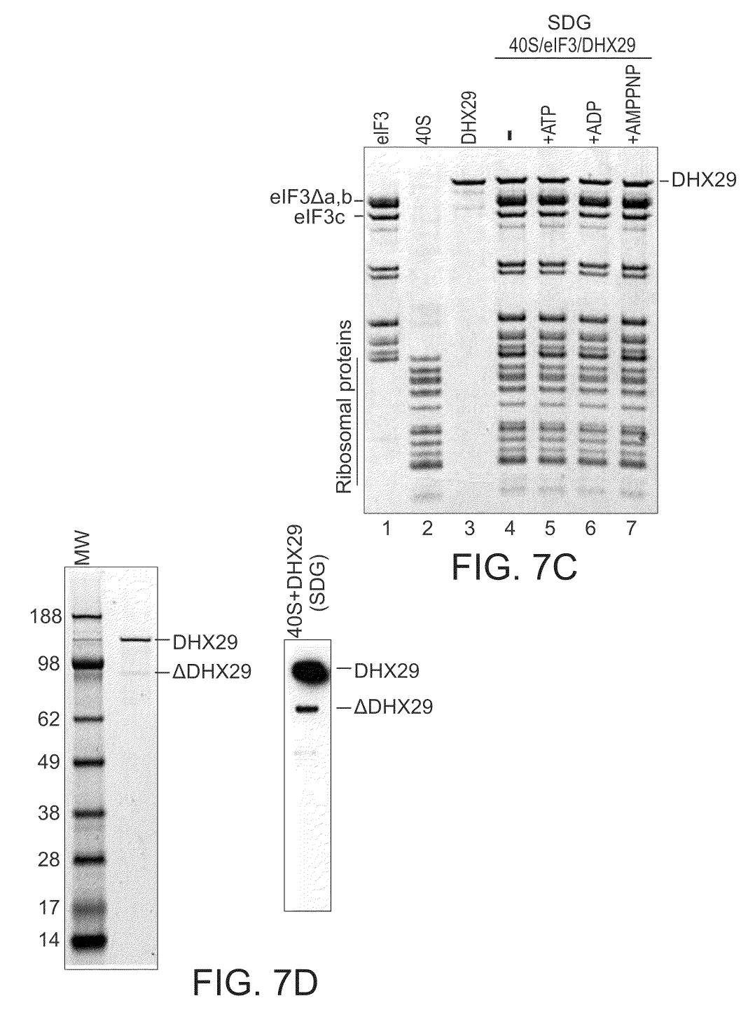

Levels of DHX29 protein may be determined by various means, such as by western blot (as in FIG. 7D), which may then be compared against levels in control cells/tissues. Using such comparative data, levels of DHX29 mRNA may be determined and compared relative to standards using quantitative RT-PCR, which are conducted using primers designed on the basis of the human DHX29 sequence (Genbank NM_{—}019030). Other comparative means may be used as desired.

The methods and uses described herein may be more clearly understood from a consideration of the non-limiting Examples provided herein.

### EXAMPLES

### 1. Efficient 48S Complex Formation on mRNAs with Structured 5′-UTRs with DHX29

Although eIF2, eIF3, eIF1, eIF1A, eIF4A, eIF4B, and eIF4F promote efficient 48S complex formation on model synthetic mRNAs comprising the β-glucuronidase (GUS) coding region and an unstructured 5′-UTR consisting of 19 CAA repeats (CAA-GUS mRNA [Seq. ID No. 76]), they did not support high level 48S complex formation on CAA-GUS Stem-3 and Stem 4 mRNAs containing more stable stems with ΔG=−18.9 and −27.6 kcal/mol, respectively. (FIG. 5B, lanes 18 and 24). Further, these eIFs also supported only very weak 48S complex assembly on neutrophil cytosolic factor 2 (NCF2) mRNA containing a 168 nt-long 5′UTR (FIG. 5C, lane 3). Finally, they did not promote 48S complex formation at all on CDC25 mRNA containing a 271 nt-long 5′-UTR (FIG. 5D, lane 2).

Extensive purification from RRL of missing factor(s) required for efficient 48S complex formation on such structured 5′-UTRs was undertaken. Purification yielded an apparently homogeneous ˜150 kDa protein, as depicted in FIG. 4, which was identified as DHX29, a putative DExH-box helicase (FIG. 2).

Experiments were conducted to determine the effect of DHX29 in an in vitro reconstituted system, which were found to increase 48S complex formation on both CAA-GUS Stem 3 and Stem-4 mRNAs (FIG. 5B, lanes 19 and 25, respectively) and on NCF2 mRNA (FIG. 5C, lane 4), and further allowed 48S complex formation on CDC25 mRNA (FIG. 5D, lane 1). DHX29 also slightly (by about 20-30%) stimulated the already efficient 48S complex formation on CAA-GUS Stem-1 [Seq. ID No. 77] and Stem-2 [Seq. ID No. 78] mRNAs (FIG. 5B, lanes 10, 15). It was discovered that moderate stimulation of 48S complex formation on Stem-containing CAA-GUS mRNAs by DHX29 occurred even in the absence of eIF4A, eIF4B, and eIF4F (FIG. 5B, lanes 3, 8, 13, 17 and 23), but was lower than by eIF4A, eIF4B and eIF4F (FIG. 5B, lanes 4, 9, 14, 18 and 24). DHX29 promoted only marginal 48S complex assembly on β-globin mRNA in the absence of eIF3F, eIF4B and eIF4A (FIG. 5A, lanes 5, 6).

### 2. Verification that 48S Complexes Assembled with DHX29 are Elongation-Competent

Experiments were conducted to verify that 48S complexes assembled with DHX29 were elongation-competent. To this end, formation of ribosomal complexes was assayed on derivatives of CAA-GUS Stem-3 and Stem-4 mRNAs encoding a MVHC tetrapeptide followed by a UAA stop codon. Addition of 60S subunits, eIF5 and eIF5B, elongation factors and aminoacylated tRNAs to 48S complexes assembled on both mRNAs with DHX29 yielded prominent toe prints +16-17 nt from the UGC Cys codon that occupies the P-site of elongating ribosomes arrested at the stop codon. This can be seen in FIG. 5E, left panel. As with 48S complexes, substantially more elongation complexes formed on both mRNAs in the presence of DHX29, assayed by toe-printing and sucrose density gradient centrifugation (FIG. 5E, right panel).

### 3. 48S Complex Formation on β-Globin mRNA

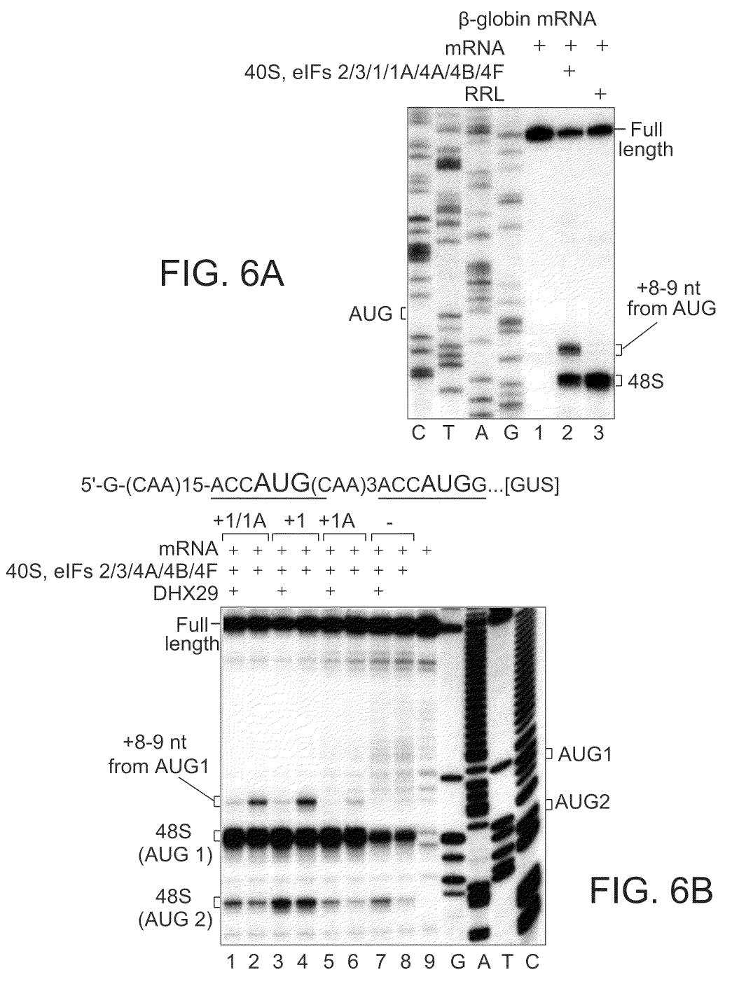

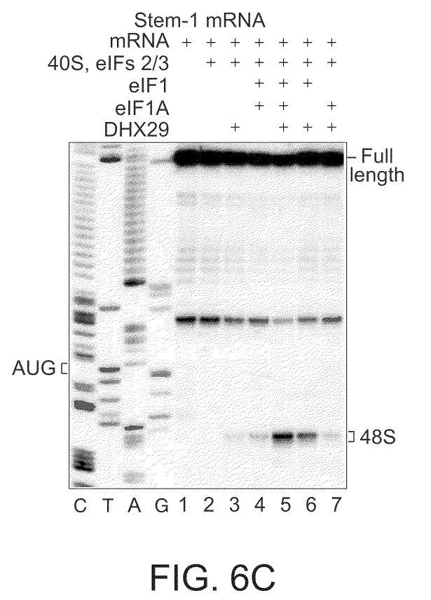

It is known that eIF2, eIF3, eIF1, eIF1A, eIF4A, eIF4B and eIF4F ensure adequate 48S complex formation on native capped β-globin mRNA. Additional toe-prints appeared +8-9 nt downstream of the AUG codon, at least as much as 30-40% of the level of the +15-17 nt toe-prints corresponding to properly assembled 48S complexes (FIG. 6A, lane 2). The +8-9 toe-prints were apparent on other mRNAs, for example on the first AUG codon of mRNA containing two AUG triplets [Seq. ID No. 79] surrounded by CAA repeats (FIG. 6B, lanes 2, 4). In contrast, 48S complexes assembled on β-globin or other mRNAs in RRL yielded toe-prints exclusively at +15-17 positions (FIG. 6A, lane 3). Appearance of the +8-9 toe-print required 40S subunits, Met-tRNA^{Met}ᵢ, eIFs and an AUG codon, thus suggesting that it corresponds to a 48S complex in which the 3′-portion of mRNA is not fixed in the 40S subunit's mRNA-binding cleft, thus allowing reverse transcriptase to penetrate further. In addition, formation of the +8-9 toe-print was also eIF1-dependent, and was exacerbated by some eIF1A mutants. Almost no such toe print was observed on the first AUG codon of mRNA with two AUG triplets in reaction mixtures lacking eIF1 (FIG. 6B, compare lanes 2, 4 and 6, 8).

DHX29 was used for formation of 48S complex formation on β-globin mRNA. Although DHX29 was found not to be absolutely essential for 48S complex formation on β-globin mRNA, it was discovered that DHX29 used in 48S complex formation on β-globin mRNA allowed a more efficient 48S complex formation. It was found that DHX29 used in this capacity suppressed the aberrant +8-9 toe-print, and had the same effect upon delayed addition to preformed initiation complexes (FIG. 5A, lanes 3, 4). Further, DHX29 also suppressed the aberrant +8-9 nt toe-print on other mRNAs, including the mRNA with two AUG triplets (FIG. 6B, lanes 1, 3).

Thus, it was determined that binding of DHX29 to ribosomal complexes induces conformational changes near the mRNA-binding cleft that accommodate the 3′-portion of mRNA. DHX29 additionally increased leaky scanning, enhancing 48S complex formation on the second AUG codon of mRNA containing two AUG triplets, irrespective of the presence of eIF1 or eIF1A (FIG. 6B, lanes 1, 3, 5, 7). In reaction mixtures lacking eIF4F, eIF4A, and eIF4B, DHX29 was found to promote low-level 48S complex formation on CAA-GUS Stem-1 even without eIF1 and eIF1A (FIG. 6C, lane 3). However, eIF1, particularly in combination with eIF1A, substantially increased initiation (FIG. 6C, lanes 5, 6).

### 4. Interactions Between DHX29 and Translational Components

Experiments were conducted to identify interactions between DHX29 and translational components that could drive DHX29 to ribosomal complexes. These experiments demonstrated that DHX29 is capable of binding stably to 40S subunits. DHX29 was also found not to bind to 60S or 80S ribosomes. Further, experiments showed that DHX29 remained associated with the 40S subunits during sucrose density gradient centrifugation (FIG. 7A, lanes 4, 5, 7). DHX29 was found to associate with 40S subunit monomers, but not to the dimers that occur in mammalian 40S subunit preparations (FIG. 7A, lanes 6, 7).

It was further discovered that DHX29 bound stably and stoichiometrically to 40S/eIF3 complexes, including those formed with (CUUU)₉ RNA. Further, DHX29 was found to bind stably to 43S complexes (FIG. 7A, lanes 8, 9). Further, DHX29 was found to bind stably and stoichiometrically to yeast 40S subunits (FIG. 7B). Experiments revealed that DHX29's ribosomal binding is nucleotide-independent (FIG. 7C), and as much DHX29 associated with 40S/eIF3 complexes in the presence or absence of ATP, ADP, or AMPPNP. In RRL, DHX29 was present in 40S-containing ribosomal complexes. Further, truncated DHX29 was prepared, which are identified as containing a ˜90-95 kDa band (FIG. 7D, left panel).

### 5. The Ribosomal Position of DHX29

To obtain insight into the ribosomal position of DHX29, experiments were conducted. Experiments revealed that the region of DHX29 responsible for ribosomal binding is located in the N-terminal two thirds of the protein. In particular, chemical and enzymatic foot-printing of 18S rRNA in 43S and 43S/DHX29 complexes were compared. It was found that DHX29 strongly protected CUC₅₂₇₋₉ and UUU₅₃₀₋₂ in the apical region of helix (h) 16 from RNase VI cleavage and CMCT modification, respectively. Further, DHX29 was found to weakly protect the neighboring A₅₂₆ from DMS modification. Finally, DHX29 did not protect G₅₃₄ on the opposite strand of the stem from RNase T1 cleavage. In eukaryotic 40S subunits, h16 is rotated towards the back of the 40S subunit, pointing into the solvent. If the observed protections resulted from direct interaction between h16 and DHX29, rather than from induced conformational changes, then DHX29 is found to bind to the 40S subunit near the mRNA entrance.

### 6. Characterization of DHX29 NTPase Activity

The NTPase activity of DHX29 was characterized to fully define the biochemical properties of DHX29. DHX29 was found to lack nucleotide specificity and hydrolyzed ATP, GTP, CTP, and UTP, which all lack the Q-motif upstream of the helicase domain that has been implicated in determining the specificity of adenine recognition by related DEAD box helicases (FIG. 8A).

DHX29's NTPase activity was strongly stimulated by 43S complexes, whereas stimulation by single-stranded RNA was low (FIGS. 8A, 8B). 18S rRNA had higher stimulatory activity than (CUUU)₉ RNA, but lower than 43S complexes (FIG. 8B). The greatest level of stimulation occurred in the presence of 43S complexes with (CUUU)₉ RNA.

eIF4A/eIF4B/eIF4F-independent 48S complex assembly on CAA-GUS Stem-1 mRNA was then investigated in the presence of DHX29 and different NTPs (FIG. 8C). 43S complexes formed with eIF2/eIF3/eIF1/eIF1A were then separated from unincorporated GTP by sucrose density gradient centrifugation and incubated with DHX29 and mRNA in the presence and absence of GTP, ATP, CTP, UTP, GMPPNP or AMPPNP. It was found that the highest stimulation of DHX29 was with GTP or ATP, and was slightly lower with CTP or UTP (FIG. 8C, lanes 4-7). It was determined that NTP hydrolysis by DHX29 may therefore be required for its activity in 48S complex formation.

### 7. Potential Helicase Activity of DHX29

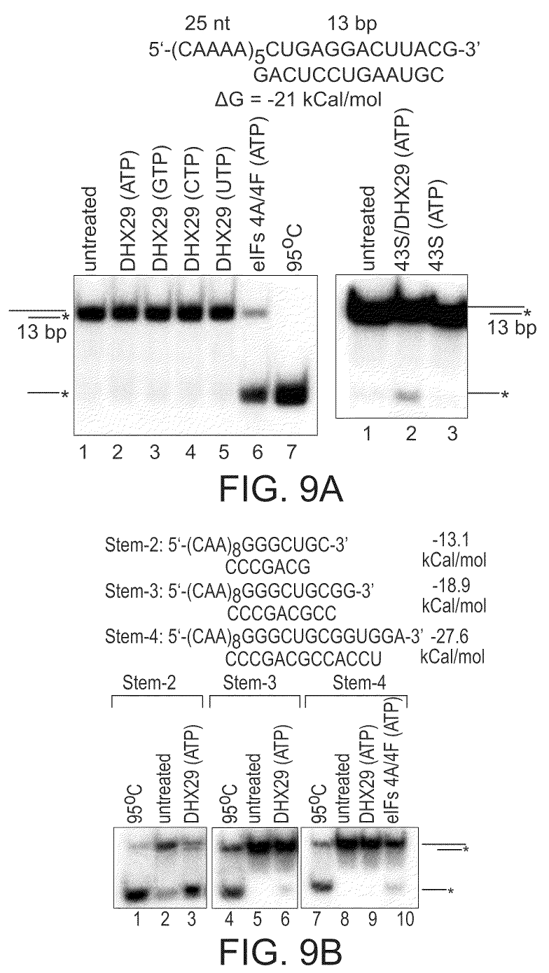

Experiments were conducted to investigate the potential helicase activity of DHX29. RNA duplexes comprising overhanging 25 nt-long 5′ or 3′-ends and 13 nt-long or 10 nt-long double stranded regions (ΔG=−21 and −14.6 kCal/mol, respectively) as well as corresponding blunt duplexes and duplexes resembling stems 2, 3, and 4 of CAA-GUS Stem-2-4 mRNAs. It was found that DHX29 did not unwind 13 nt-long duplexes with overhanging 5′- or 3′-ends in the presence of NTP, whereas unwinding by eIF4A/eIF4F was efficient (FIG. 9A, left panel). There was found weak unwinding of these duplexes by isolated 43S/DHX29 complexes (FIG. 9A, right panel, lane 2). Additionally, there was found marginal unwinding (i.e., less than 5%) by DHX29 of 10 nt-long duplexes with overhanging ends. DHX29 was found to unwind Stem-2 duplex (FIG. 9B, lane 3). Further, Stem-3 duplex unwinding by DHX29 was marginal (FIG. 9B, lane 6).

### 8. DHX29 Participation in Multiple Rounds of 48S Complex Formation

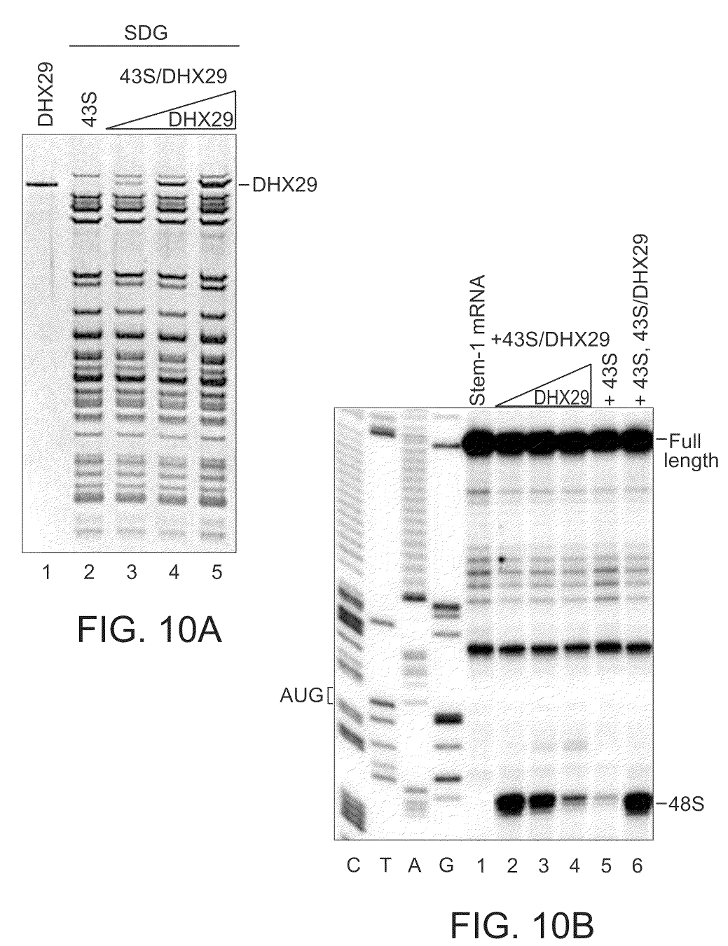

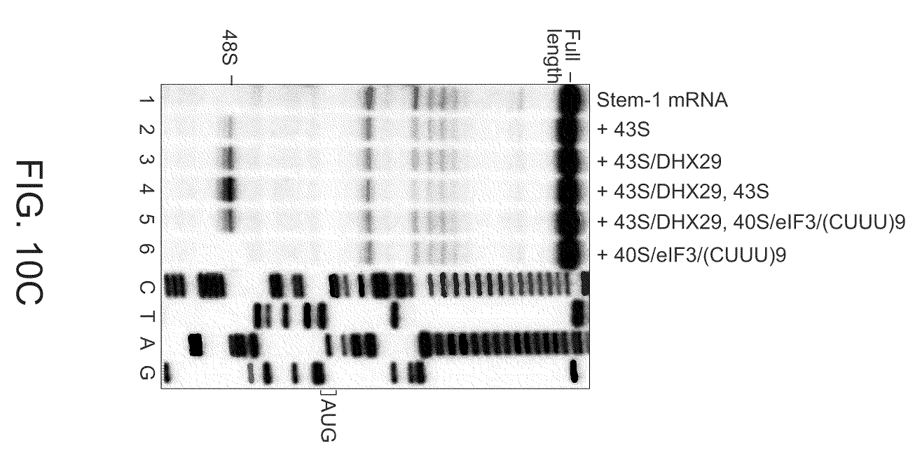

DHX29 was found to stimulate 48S complex formation most strongly when it was present in substoichiometric amounts relative to 43S complexes. The most active in 48S complex assembly on GAA-GUS Stem-1 mRNA were sucrose density gradient-purified 43 S/DHX29 complexes having a ratio of 43S to DHX29 of about 10:1 (FIG. 10A, lane 3). Complexes with 43S:DHX29 ratios of from about 2:1 to about 1:1 were found to be progressively less active (FIG. 10A, lanes 4, 5).

A mixture of DHX29-free 43S complexes and 43S/DHX29-saturated 43S complexes that individually had low activities were found to together promote very efficient 48S complex formation (FIG. 10B, lanes 4, 5). As such, a proportion of DHX29 may be inactive, but the DHX29 from active 43S/DHX29 complexes may have beneficial activities, including being able to dissociate from ribosomal complexes and participating in new rounds of initiation.

In an alternative, it was found that stimulation of 48S complex formation by DHX29 may require dissociation from the 40S subunit at a point in the process before the 48S complex is formed. In this embodiment, the excess of free 43S complexes would ensure rebinding of dissociated DHX29 to a new 43S complex. To investigate this embodiment, DHX29-saturated 43S complexes were mixed with purified complex of 40S, eIF3 and (CUUU)₉. The complex of 40S, eIF3 and (CUUU)₉ were found to not stimulate 48S complex formation by 43S/DHX29 complexes (FIG. 10C, lanes 3, 5).

### 9. Influence of DHX29 on 48S Complex Formation During IRES-Mediated Initiation

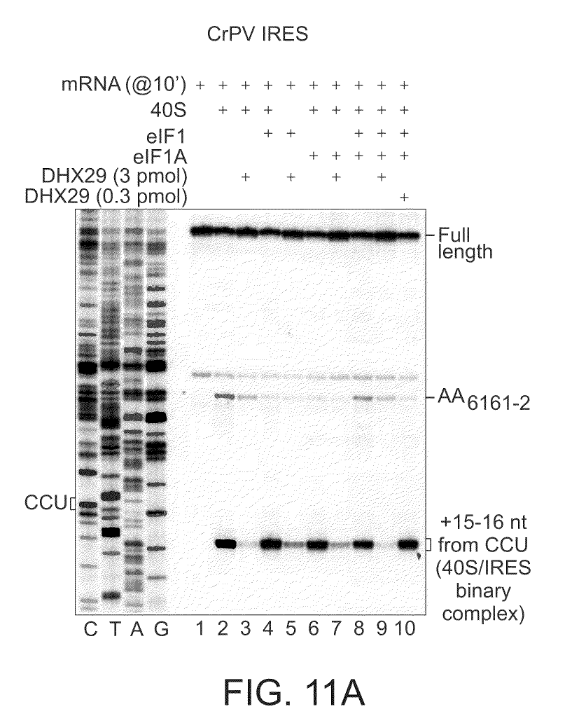

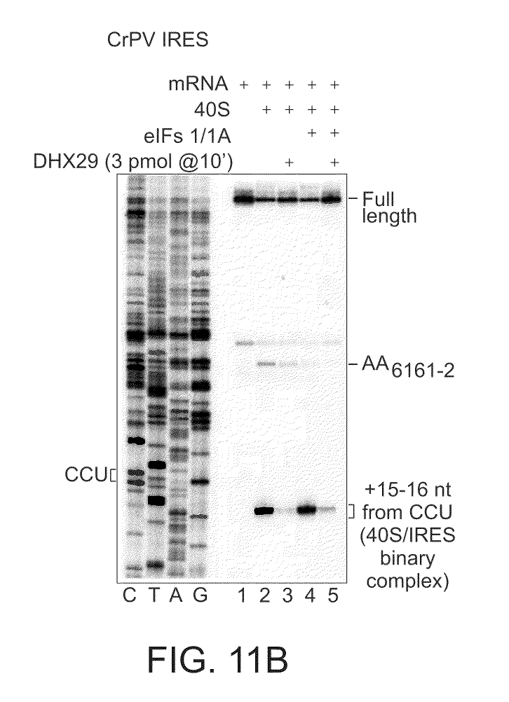

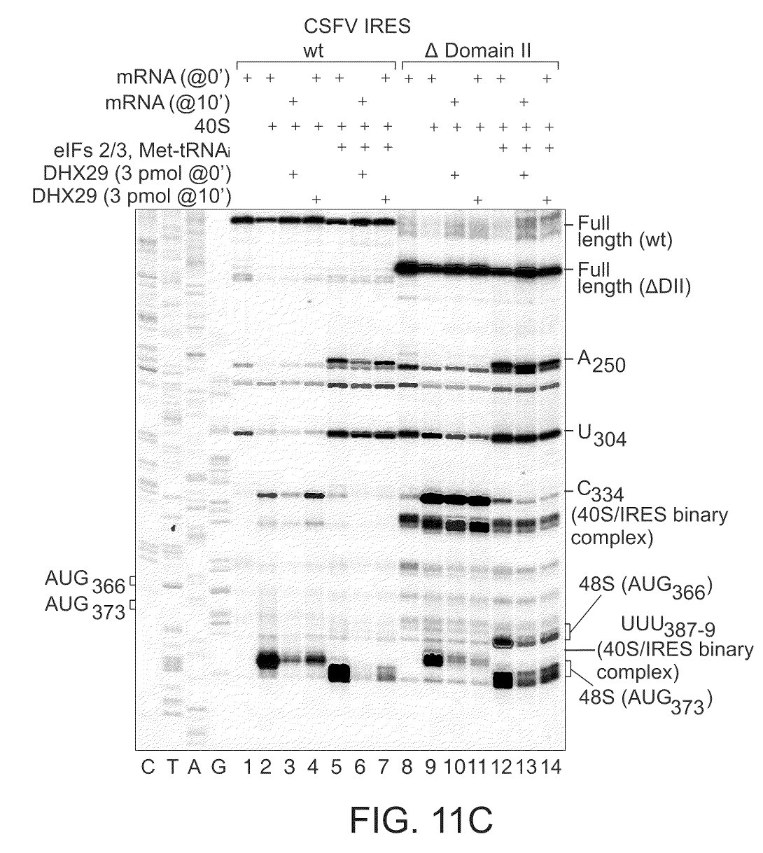

As set forth above, DHX29 may be used in aiding 48S complex formation during IRES-mediated initiation if this process involves internal ribosomal entry followed by scanning, but may impair initiation if this process involves direct binding of the ribosome to the initation codon, for example on the intergenic region (IGR) IRES of Dicstroviruses such as Cricket paralysis virus (CrPV) and the Heptatitis C virus (PCV)-like IRESs of vlaviviruses such as Classical swine fever virus (CSFV) and picornaviruses such as Simian Picornavirus type 9 (SPV9). Experiments were conducted to determine the influence of DHX29 on such 48S complex formation. Generally, binding of the CrPV IRES [Seq. ID No. 80] to 40S subunits yields two sets of toe-prints, one corresponding to the leading edge of the 40S subunit +15-16 nt from the P-site CCU codon (at AG₆₂₂₈₋₉), and one corresponding to a second IRES-40S subunit interaction (at AA₆₁₆₁₋₂). When present in stoichiometric amounts relative to 40S subunits, DHX29 was found to almost abrogate the toe-prints at AG₆₂₂₈₋₉ irrespective of whether DHX29 was added before CrPV IRES mRNA (FIG. 11A) or to preassembled IRES/40S complexes (FIG. 11B).

Binding of CSFV IRES [Seq. ID No. 81] to 40S subunits also yields two sets of toe-prints, the first corresponding to the leading edge of the 40S subunit +15-17 nt from the P-site AUG codon (at UUU₃₈₇₋₉) and a second corresponding to a contact of the 40S subunit with the pseudoknot of the IRES (at C₃₃₄). Again, DHX29 was found to strongly reduce the toe-prints at UUU₃₈₇₋₉ in 40S/CSFV IRES complexes, irrespective of when it was added (FIG. 11C). DHX29 was found to have less effect on toe-prints corresponding to 40S/IRES contacts outside the mRNA-binding cleft than on toe-prints at the leading edge of the bound 40S subunit.

Even upon delayed addition, DHX29 was found to abrogate toe-prints corresponding to 48S complexes assembled on the CSFV IRES in the presence of eIF2, eIF3 and Met-tRNA^{Met}ᵢ (FIG. 11C). Deletion of IRES domain II [Seq. ID No. 82] was found to eliminate the sensitivity of 48S complexes to dissociation by eIF1. Although deletion of domain II did not completely suppress the dissociating effect of DHX29, 48S complexes assembled on the IRES lacking domain II were less sensitive to DHX29 than complexes assembled on the wt IRES (FIG. 11C, lanes 5-7 and 12-14). 48S complexes assembled on the HCV-like IRES of Simian picornavirus type 9, which are much more resistant to dissociation by eIF1, were resistant to dissociation by DHX29.

### 10. Purification of Native DHX29

DHX29 was purified from the 0-40% ammonium sulphate precipitation fraction of the 0.5M KCl ribosomal salt wash from 2 liters of rabbit reticulocyte lysate (RRL). The pellet was resuspended in buffer A (20 mM Tris-HCl, pH 7.5, 10% glycerol, 2 mM DTT, 0.1 mM EDTA) containing 100 mM KCl and applied to a DEAE (D52) column equilibrated with buffer A+100 mM KCl. The fraction containing DHX29 was eluted in the flow-through fraction with buffer A+100 mM KCl. This fraction was applied to a phosphocellulose (P11) column equilibrated with buffer A+100 mM KCl. Step elution was done with buffer A containing 100, 200, 300, 400 and 500 mM KCl. DHX29 eluted at 300-400 mM KCl. This fraction was dialyzed overnight against buffer B (20 mM HEPES, pH 7.5, 5% glycerol, 2 mM DTT, 0.1 mM EDTA) containing 100 mM KCl and then applied to a FPLC MonoS HR 5/5 column. Fractions were collected across a 100-500 mM KCl gradient. DHX29 eluted at ±300 mM KCl. DHX29-containing fractions were dialyzed overnight against buffer C (20 mM Tris-HCl, pH 7.5, 5% glycerol, 2 mM DTT, 0.1 mM EDTA) containing 100 mM KCl and then applied to a FPLC MonoQ HR 5/5 column. Fractions were collected across a 100-500 mM KCl gradient. DHX29 eluted at ˜250 mM KCl. DHX29-containing fractions were dialyzed overnight against buffer containing 20 mM Tris-HCl, pH 7.5, 5% glycerol and 100 mM KCl, then diluted 5-fold with 20 mM phosphate buffer, pH 7.5 with 5% glycerol and applied to a hydroxyapatite column pre-equilibrated in the same phosphate buffer. Fractions were collected across a 20-500 mM phosphate buffer gradient. Apparently homogenous DHX29 eluted at ˜300 mM phosphate buffer. The identity of DHX29 was confirmed by LC-nanospray tandem mass spectrometry of peptides derived by in-gel tryptic digestion at the Rockefeller University Proteomics Resource Center.

It should be understood that various alternatives to the embodiments of the present invention described herein can be employed in practicing the present invention. It is intended that the following claims define the scope of the present invention and that structures and methods within the scope of these claims and their equivalents be covered entirely.

TABLE 3
Listings of Sequences in the Present Application

| | | |
|---|---|---|
| Seq. ID | | Description of |
| No. | Deduced Sequence | Sequence |

| | | |
|---|---|---|
| 1 | mggknkkhka paaavvraav sasraksaea giageaqskk pvsrpataaa | DHX29 |
| | aaagsreprv kqgpkiysfn stndssgpan ldksilkvvi nnkleqriig | |
| | vinehkkqnn dkgmisgrlt akklqdlyma lqafsfktkd iedamtntll | |
| | yggdlhsald wlclnlsdda lpegfsqefe eqqpksrpkf qspqiqatis | |
| | pplqpktkty eedpkskpkk eeknmevnmk ewilryaeqq | |
| | neeeknensk sleeeekfdp nerylhlaak lldakeqaat fkleknkqgq | |
| | keaqekirkf qremetledh pvfnpamkis hqqnerkkpp vategesaln | |
| | fnlfeksaaa teeekdkkke phdvrnfdyt arswtgkspk qflidwvrkn | |
| | lpkspnpsfe kvpvgrywkc rvrviksedd vlvvcptilt edgmqaqhlg | |
| | atlalyrlvk gqsvhqllpp tyrdvwlews daekkreeln kmetnkprdl | |
| | fiakllnklk qqqqqqqqhs enkrensedp eeswenlvsd edfsalsles | |
| | anvedlepvr nlfrklqstp kyqkllkerq qlpvfkhrds ivetlkrhrv | |
| | vvvagetgsgkstqvphfll edlllnewea skcnivctqp rrisavslan | |
| | rvcdelgcen gpggrnslcg yqirmesrac estrllyctt gvllrklqed | |
| | gllsnvshvi vdevhersvq sdflliilke ilqkrsdlhl ilmsatvdse | |
| | kfstyfthcp ilrisgrsyp vevfhledii eetgfvlekd seycqkflee | |
| | eeevtinvts kaggikkyqe yipvqtgaha dlnpfyqkys srtqhailym | |
| | nphkinldli lellayldks pqfrniegav liflpglahi qqlydllsnd | |
| | rrfyserykv ialhsilstq dqaaaftlpp pgvrkivlat niaetgitip | |
| | dvvfvidtgr tkenkyhess qmsslvetfv skasalqrqg ragrvrdgfc | |
| | frmytrerfe gfmdysvpei lrvpleelcl himkcnlgsp edflskaldp | |
| | pqlqvisnam nllrkigace lnepkltplg qhlaalpvnv kigkmlifga | |
| | ifgcldpvat laavmteksp fttpigrkde adlaksalam adsdhltiyn | |
| | aylgwkkarq eggyrseity crrnflnrts lltledvkqe liklvkaagf | |
| | sssttstswe gnrasqtlsf qeiallkavl vaglydnvgk iiytksvdvt | |
| | eklaciveta qgkaqvhpss vnrdlqthgw llyqekirya rvylrettli | |
| | tpfpvllfgg dievqhrerl lsidgwiyfq apvkiavifk qlrvlidsvl | |
| | rkklenpkms lendkilqii teliktenn | |
| 2 | msgaldvlqm keedvlkfla agthlggtnl dfqmeqyiyk rksdgiyiin | Ribosomal |
| | lkrtweklll aaraivaien padvsvissr ntgqravlkf aaatgatpia | Protein rpSA |
| | grftpgtftn qiqaafrepr llvvtdprad hqplteasyv nlptialcnt | |
| | dsplryvdia ipcnnkgahs vglmwwmlar evlrmrgtis | |
| | rehpwevmpd lyfyrdpeei ekeeqaaaek avtkeefqge wtapapefta | |
| | tqpevadwse gvqvpsvpiq qfptedwsaq patedwsaap taqatewvga | |
| | ttdws | |
| 3 | maddagaagg pggpggpgmg nrggfrggfg sgirgrgrgr grgrgrgrga | Ribosomal |
| | rggkaedkew mpvtklgrlv kdmkikslee iylfslpike seiidfflga | Protein rpS2 |
| | slkdevlkim pvqkqtragq rtrfkafvai gdynghvglg vkcskevata | |
| | irgaiilakl sivpvrrgyw gnkigkphtv pckvtgrcgs vlvrlipapr | |
| | gtgivsapvp kkllmmagid dcytsargct atlgnfakat fdaisktysy | |
| | ltpdlwketv ftkspyqeft dhlvkthtrv svqrtqapav att | |
| 4 | mavqiskkrk fvadgifkae lnefltrela edgysgvevr vtptrteiii | Ribosomal |
| | latrtqnvlg ekgrrirelt avvqkrfgfp egsvelyaek vatrglcaia | protein rpS3 |
| | qaeslrykll gglavrracy gvlrfimesg akgcevvvsg klrgqraksm | |
| | kfvdglmihs gdpvnyyvdt avrhvllrqg vlgikvkiml pwdptgkigp | |
| | kkplpdhvsi vepkdeilpt tpiseqkggk peppampqpv pta | |
| 5 | mavgknkrlt kggkkgakkk vvdpfskkdw ydvkapamfn | Ribosomal |
| | irnigktlvt rtqgtkiasd glkgrvfevs ladlqndeva frkfklited | protein rpS3a |
| | vqgkncltnf hgmdltrdkm csmvkkwqtm ieahvdvktt dgyllrlfcv | |
| | gftkkrnnqi rktsyaqhqq vrqirkkmme imtrevqtnd lkevvnklip | |
| | dsigkdieka cqsiyplhdv fvrkvkmlkk pkfelgklme lhgegsssgk | |
| | atgdetgakv eradgyeppv qesv | |
| 6 | margpkkhlk rvaapkhwml dkltgvfapr pstgphklre clpliiflrn | Ribosomal |
| | rlkyaltgde vkkicmqrfi kidgkvrtdi typagfmdvi sidktgenfr | protein rpS4X |
| | liydtkgrfa vhritpeeak yklckvrkif vgtkgiphlv thdartiryp | |
| | dplikvndti qidletgkit dfikfdtgnl cmvtgganlg rigvitnrer | |
| | hpgsfdvvhv kdangnsfat rlsnifvigk gnkpwislpr gkgirltiae | |
| | erdkrlaakq ssg | |
| 7 | mtewetaapa vaetpdiklf gkwstddvqi ndislqdyia vkekyakylp | Ribosomal |
| | hsagryaakr frkaqcpive rltnsmmmhg rnngkklmtv rivkhafeii | protein rpS5 |
| | hlltgenplq vlvnaiinsg predstrigr agtvrrqavd vsplrrvnqa | |
| | iwllctgare aafrniktia ecladelina akgssnsyai kkkdelerva ksnr | |
| 8 | mklnisfpat gcqklievdd erklrtfyek rmatevaada lgeewkgyvv | Ribosomal |
| | risggndkqg fpmkqgvlth grvrlllskg hscyrprrtg erkrksvrgc | protein rpS6 |
| | ivdanlsvln lvivkkgekd ipgltdttvp rrlgpkrasr irklfnlske | |
| | ddvrqyvvrk plnkegkkpr tkapkiqrlv tprvlqhkrr rialkkqrtk | |
| | knkeeaaeya kllakrmkea kekrqeqiak rrrlsslras tsksessqk | |
| 9 | mfsssakivk pngekpdefe sgisqallel emnsdlkaql relnitaake | Ribosomal |
| | ievgggrkai iifvpvpqlk sfqkiqvrlv relekkfsgk hvvfiaqrri | protein rpS7 |
| | lpkptrksrt knkqkrprsr tltavhdail edlvfpseiv gkrirvkldg | |
| | srlikvhldk aqqnnvehkv etfsgvykkl tgkdvnfefp efql | |
| 10 | mgisrdnwhk rrktggkrkp yhkkrkyelg rpaantkigp rrihtvrvrg | Ribosomal |
| | gnkkyralrl dvgnfswgse cctrktriid vvynasnnel vrtktlvknc | protein rpS8 |
| | ivlidstpyr qwyeshyalp lgrkkgaklt peeeeilnkk rskkiqkkyd | |
| | erkknakiss lleeqfqqgk llaciasrpg qcgradgyvl egkelefylr | |
| | kikarkgk | |
| 11 | mpvarswvcr ktyvtprrpf eksrldqelk ligeyglrnk revwrvkftl | Ribosomal |
| | akirkaarel ltldekdprr lfegnallrr lvrigvldeg kmkldyilgl | protein rpS9 |
| | kiedflerrl qtqvfklgla ksihharvli rqrhirvrkq vvnipsfivr | |
| | ldsqkhidfs lrspygggrp grvkrknakk gqggagagdd eeed | |
| 12 | mlmpkknria iyellfkegv mvakkdvhmp khpeladknv | Ribosomal |
| | pnlhvmkamq slksrgyvke qfawrhfywy ltnegiqylr dylhlppeiv | protein rpS10 |
| | patlrrsrpe tgrprpkgle gerparltrg eadrdtyrrs avppgadkka | |
| | eagagsatef qfrggfgrgr gqppq | |
| 13 | madiqteray qkqptifqnk krvllgetgk eklpryykni glgfktpkea | Ribosomal |
| | iegtyidkkc pftgnvsirg rilsgvvtkm kmqrtivirr dylhyirkyn | protein rpS11 |
| | rfekrhknms vhlspcfrdv qigdivtvge crplsktvrf nvlkvtkaag | |
| | tkkqfqkf | |
| 14 | maeegiaagg vmdvntalqe vlktalihdg largireaak aldkrqahlc | Ribosomal |
| | vlasncdepm | protein rpS12 |
| | yvklvealca ehqinlikvd dnkklgewvg lckidregkp rkvvgcscvv | |
| | vkdygkesqa kdvieeyfkc kk | |
| 15 | mgrmhapgkg lsqsalpyrr svptwlklts ddvkeqiykl akkgltpsqi | Ribosomal |
| | gvilrdshgv aqvrfvtgnk ilrilkskgl apdlpedlyh likkavavrk | protein rpS13 |
| | hlernrkdkd akfrlilies rihrlaryyk tkrvlppnwk yesstasalv a | |
| 16 | maprkgkekk eeqvislgpq vaegenvfgv chifasfndt fvhvtdlsgk | Ribosomal |
| | eticrvtggm kvkadrdess pyaamlaaqd vaqrckelgi talhiklrat | protein rpS14 |
| | ggnrtktpgp gaqsalrala rsgmkigrie dvtpipsdst rrkggrrgrr l | |
| 17 | maeveqkkkr tfrkftyrgv dldqlldmsy eqlmqlysar qrrrlnrglr | Ribosomal |
| | rkqhsllkrl rkakkeappm ekpevvkthl rdmiilpemv gsmvgvyngk | protein rpS15 |
| | tfnqveikpe mighylgefs itykpvkhgr pgigathssr fiplk | |
| 18 | mvrmnvlada lksinnaekr gkrqvlirpc skvivrfltv mmkhgyigef | Ribosomal |
| | eiiddhragk ivvnltgrln kcgvisprfd vqlkdlekwq nnllpsrqfg | protein rpS15A |
| | fivlttsagi mdheearrkh tggkilgfff | |
| 19 | mpskgplqsv qvfgrkktat avahckrgng likvngrple mieprtlqyk | Ribosomal |
| | llepvlllgk erfagvdirv rvkggghvaq iyairqsisk alvayyqkyv | protein rpS16 |
| | deaskkeikd iliqydrtll vadprrcesk kfggpgarar ygksyr | |
| 20 | mgrvrtktvk kaarviieky ytrlgndfht nkrvceeiai ipskklrnki | Ribosomal |
| | agyvthlmkr iqrgpvrgis iklqeeerer rdnyvpevsa ldqeiievdp | protein rpS17 |
| | dtkemlklld fgslsnlqvt qptvgmnfkt prgpv | |
| 21 | mslvipekfq hilrvlntni dgrrkiafai taikgvgrry ahvvlrkadi | Ribosomal |
| | dltkragelt edeverviti mqnprqykip dwflnrqkdv kdgkysqvla | protein rpS18 |
| | ngldnklred lerlkkirah rglrhfwglr vrgqhtkttg rrgrtvgvsk kk | |
| 22 | mpgvtvkdvn qqefvralaa flkksgklkv pewvdtvkla khkelapyde | Ribosomal |
| | nwfytraast arhlylrgga gvgsmtkiyg grqrngvmps hfsrgsksva | protein rpS19 |
| | rrvlqalegl kmvekdqdgg rkltpqgqrd ldriagqvaa ankkh | |
| 23 | mafkdtgktp vepevaihri ritltsrnvk slekvcadli rgakeknlkv | Ribosomal |
| | kgpvrmptkt lrittrktpc gegsktwdrf qmrihkrlid lhspseivkq | protein rpS20 |
| | itsisiepgv evevtiada | |
| 24 | mqndagefvd lyvprkcsas nriigakdha siqmnvaevd kvtgrfngqf | Ribosomal |
| | ktyaicgair rmgesddsil rlakadgivs knf | protein rpS21 |
| 25 | mgkcrglrta rklrshrrdq kwhdkqykka hlgtalkanp fggashakgi | Ribosomal |
| | vlekvgveak qpnsairkcv rvqlikngkk itafvpndgc lnfieendev | protein rpS23 |
| | lvagfgrkgh avgdipgvrf kvvkvanvsl lalykgkker prs | |
| 26 | mndtvtirtr kfmtnrllqr kqmvidvlhp gkatvpktei reklakmykt | Ribosomal |
| | tpdvifvfgf rthfgggktt gfgmiydsld yakknepkhr larhglyekk | protein rpS24 |
| | ktsrkqrker knrmkkvrgt akanvgagkk pke | |
| 27 | mppkddkkkk dagksakkdk dpvnksggka kkkkwskgkv | Ribosomal |
| | rdklnnlvlf dkatydklck evpnyklitp avvserlkir gslaraalqe | protein rpS25 |
| | llskgliklv skhraqviyt rntkggdapa ageda | |
| 28 | mtkkrrnngr akkgrghvqp irctncarcv pkdkaikkfv irniveaaav | Ribosomal |
| | rdiseasvfd ayvlpklyvk lhycvscaih skvvrnrsre arkdrtpppr | protein rpS26 |
| | frpagaaprp ppkpm | |
| 29 | mplakdllhp speeekrkhk kkrlvqspns yfmdvkcpgc ykittvfsha | Ribosomal |
| | qtvvlcvgcs tvlcqptggk arltegcsfr rkqh | protein rpS27 |
| 30 | akkrkkksyt tpkknkhkrk kvklavlkyy kvdengkisr lrrecpsdec | Ribosomal |
| | gagvfmashf drhycgkccl tycfnkpedk | protein rpS27A |
| 31 | mdtsrvqpik larvtkvlgr tgsqgqctqv rvefmddtsr siirnvkgpv | Ribosomal |
| | regdvltlle serearrlr | protein rpS28 |
| 32 | mghqqlywsh prkfgqgsrs crvcsnrhgl irkyglnmcr qcfrqyakdi | Ribosomal |
| | gfikld | protein rpS29 |
| 33 | kvhgslarag kvrgqtpkva kqekkkkktg rakrrmqynr rfvnvvptfg | Ribosomal |
| | kkkgpnans | protein rpS30 |
| 34 | tacctggttg atcctgccag tagcatatgc ttgtctcaaa gattaagcca | H. sapiens 18S |
| | tgcatgtctg agtacgcacg gccggtacag tgaaactgcg aatggctcat | |
| | taaatcagtt atggttcctt tggtcgctcg ctcctctcct acttggataa | |
| | ctgtggtaat tctagagcta atacatgccg acgggcgctg acccccttcg | |
| | cgggggggat gcgtgcattt atcagatcaa aaccaacccg gtcagcccct | |
| | ctccggcccc ggccgggggg cgggcgccgg cggctttggt gactctagat | |
| | aacctcgggc cgatcgcacg ccccccgtgg cggcgacgac ccattcgaac | |
| | gtctgcccta tcaactttcg atggtagtcg ccgtgcctac catggtgacc | |
| | acgggtgacg gggaatcagg gttcgattcc ggagagggag cctgagaaac | |
| | ggctaccaca tccaaggaag gcagcaggcg cgcaaattac ccactcccga | |
| | cccggggagg tagtgacgaa aaataacaat acaggactct ttcgaggccc | |
| | tgtaattgga atgagtccac tttaaatcct ttaacgagga tccattggag | |
| | ggcaagtctg gtgccagcag ccgcggtaat tccagctcca atagcgtata | |
| | ttaaagttgc tgcagttaaa aagctcgtag ttggatcttg ggagcgggcg | |
| | ggcggtccgc cgcgaggcga gccaccgccc gtccccgccc cttgcctctc | |
| | ggcgccccct cgatgctctt agctgagtgt cccgcggggc ccgaagcgtt | |
| | tactttgaaa aaattagagt gttcaaagca ggcccgagcc gcctggatac | |
| | cgcagctagg aataatggaa taggaccgcg gttctatttt gttggttttc | |
| | ggaactgagg ccatgattaa gagggacggc cgggggcatt cgtattgcgc | |
| | cgctagaggt gaaattcttg gaccggcgca agacggacca gagcgaaagc | |
| | atttgccaag aatgttttca ttaatcaaga acgaaagtcg gaggttcgaa | |
| | gacgatcaga taccgtcgta gttccgacca taaacgatgc cgaccggcga | |
| | tgcggcggcg ttattcccat gacccgccgg gcagcttccg ggaaaccaaa | |
| | gtctttgggt tccgggggga gtatggttgc aaagctgaaa cttaaaggaa | |
| | ttgacggaag ggcaccacca ggagtggagc ctgcggctta atttgactca | |
| | acacgggaaa cctcacccgg cccggacacg gacaggattg acagattgat | |
| | agctctttct cgattccgtg ggtggtggtg catggccgtt cttagttggt | |
| | ggagcgattt gtctggttaa ttccgataac gaacgagact ctggcatgct | |
| | aactagttac gcgacccccg agcggtcggc gtcccccaac ttcttagagg | |
| | gacaagtggc gttcagccac ccgagattga gcaataacag gtctgtgatg | |
| | cccttagatg tccggggctg cacgcgcgct acactgactg gctcagcgtg | |
| | tgcctaccct acgccggcag gcgcgggtaa cccgttgaac cccattcgtg | |
| | atggggatcg gggattgcaa ttattcccca tgaacgagga attcccagta | |
| | agtgcgggtc ataagcttgc gttgattaag tccctgccct ttgtacacac | |
| | cgcccgtcgc tactaccgat tggatggttt agtgaggccc tcggatcggc | |
| | cccgccgggg tcggcccacg gccctggcgg agcgctgaga agacggtcga | |
| | acttgactat ctagaggaag taaaagtcgt aacaaggttt ccgtaggtga | |
| | acctgcggaa ggatcatta | |
| 35 | sleeeekfdpner | Truncated |
| | | peptide from |
| | | DHX29 (251-263) |
| 36 | spnpsfek | Truncated |
| | | peptide from |
| | | DHX29 (394-401) |
| 37 | dlfiak | Truncated |
| | | peptide from |
| | | DHX29 (489-494) |
| 38 | vvvvagetgsgk | Truncated |
| | | peptide from |
| | | DHX29 (590-601) |
| 39 | asqtlsfqeiallk | Truncated |
| | | peptide from |
| | | DHX29 (1204-1217) |
| 40 | lacivetaqgk | Truncated |
| | | peptide from |
| | | DHX29 (1243-1253) |
| 41 | vlidsvlr | Truncated |
| | | peptide from |
| | | DHX29 (1334-1341) |
| 42 | ilqiitelik | Truncated |
| | | peptide from |
| | | DHX29 (1356-1365) |
| 43 | iigvinehk | Truncated |
| | | peptide from |
| | | DHX29 (98-106) |
| 44 | sleeeekfdpner | Truncated |
| | | peptide from |
| | | DHX29 (251-263) |
| 45 | vvvvagetgsgk | Truncated |
| | | peptide from |
| | | DHX29 (590-601) |
| 46 | vcdelgcengpggr | Truncated |
| | | peptide from |
| | | DHX29 (642-655) |
| 47 | nslcgyqir | Truncated |
| | | peptide from |
| | | DHX29 (656-664) |
| 48 | acacuugcuuuugacacaacuguguuuacuugcaaucccccaaaacagac | Messenger |
| | agaauggugcaucuguccagugaggagaagucugcggucacugcccugug | RNA for beta- |
| | gggcaaggugaauguggaagaaguugguggugaggcccugggcaggcugc | globin |
| | ugguugucuacccauggacccagagguucuucgaguccuuuggggaccug | |
| | uccucugcaaaugcuguuaugaacaauccuaaggugaaggcucauggcaa | |
| | gaaggugcuggcugccuucagugagggucugagucaccuggacaaccuca | |
| | aaggcaccuuugcuaagcugagugaacugcacugugacaagcugcacgug | |
| | gauccugagaacuucaggcuccugggcaacgugcugguuauugugcuguc | |
| | ucaucauuuuggcaaagaauucacuccucaggugcaggcugccuaucaga | |
| | aggugguggcugguguggccaaugcccuggcucacaaauaccacugagau | |
| | cuuuuucccucugccaaaaauuauggggacaucaugaagccccuugagca | |
| | ucugacuucuggcuaauaaaggaaauuuauuuucauugc | |
| 49 | msasqdsrsr dngpdgmepe gviesnwnei vdsfddmnls esllrgiyay | Eukaryotic |
| | gfekpsaiqq railpcikgy dviaqaqsgt gktatfaisi lqqieldlka | translation |
| | tqalvlaptr elaqqiqkvv malgdymgas chaciggtnv raevqklqme | initiation factor |
| | aphiivgtpg rvfdmlnrry lspkyikmfv ldeademlsr gfkdqiydif | 4A isoform 1 |
| | qklnsntqvv llsatmpsdv levtkkfmrd pirilvkkee ltlegirqfy | [Homo |
| | invereewkl dtlcdlyetl titqavifin trrkvdwlte kmhardftvs | sapiens] |
| | amhgdmdqke rdvimrefrs gssrvlittd llargidvqq vslvinydlp | |
| | tnrenyihri grggrfgrkg vainmvteed krtlrdietf yntsieempl | |
| | nvadli | |
| 50 | maasakkknk kgktisltdf laedggtggg styvskpvsw adetddlegd | Eukaryotic |
| | vsttwhsndd dvyrappidr silptapraa repnidrsrl pksppytafl | translation |
| | gnlpydvtee sikeffrgln isavrlprep snperlkgfg yaefedldsl | initiation factor |
| | lsalslnees lgnrrirvdv adqaqdkdrd drsfgrdrnr dsdktdtdwr | 4B [Homo |
| | arpatdsfdd ypprrgddsf gdkyrdryds dryrdgyrdg yrdgprrdmd | sapiens] |
| | ryggrdrydd rgsrdydrgy dsrigsgrra fgsgyrrddd yrgggdryed | |
| | rydrrddrsw ssrddysrdd yrrddrgppq rpklnlkprs tpkeddssas | |
| | tsqstraasi fggakpvdta arereveerl qkeqeklqrq ldepklerrp | |
| | rerhpswrse etqerersrt gsessqtgts ttssrnarrr esekslenet | |
| | lnkeedchsp tskppkpdqp lkvmpapppk enawvkrssn pparsqssdt | |
| | eqqsptsggg kvapaqpsee gpgrkdenkv dgmnapkgqt | |
| | gnssrgpgdg gnrdhwkesd rkdgkkdqds rsapepkkpe enpaskfssa | |
| | skyaalsvdg edenegedya e | |
| 51 | matvepettp tpnpptteee ktesnqevan pehyikhplq nrwalwffkn | Eukaryotic |
| | dksktwqanl rliskfdtve dfwalynhiq lssnlmpgcd yslfkdgiep | translation |
| | mwedeknkrg grwlitlnkq qrrsdldrfw letllclige sfddysddvc | initiation factor |
| | gavvnvrakg dkiaiwttec enreavthig rvykerlglp pkivigyqsh | 4E [Homo |
| | adtatksgst tknrfvv | sapiens] |
| 52 | mnkapqstgp ppapspglpq pafppgqtap vvfstpqatq mntpsqprqh | Eukaryotic |
| | fypsraqpps saasrvqsaa parpgpaahv ypagsqvmmi psqisypasq | translation |
| | gayyipgqgr styvvptqqy pvqpgapgfy pgasptefgt yagayypaqg | initiation factor |
| | vqqfptgvap apvlmnqppq iapkrerkti rirdpnqggk diteeimsga | 4G1 isoform 1 |
| | rtastptppq tggglepqan getpqvaviv rpddrsqgai iadrpglpgp | [Homo |
| | ehspsesqps spsptpspsp vlepgsepnl avlsipgdtm ttiqmsvees | sapiens] |
| | tpisretgep yrlspeptpl aepilevevt lskpvpesef sssplqaptp | |
| | lashtveihe pngmvpsedl epevesspel apppacpses pvpiaptaqp | |
| | eellngapsp pavdlspvse peeqakevta smapptipsa tpatapsats | |
| | paqeeemeee eeeeegeage ageaesekgg eellppestp ipanlsqnle | |
| | aaaatqvavs vpkrrrkike lnkkeavgdl ldafkeanpa vpevenqppa | |
| | gsnpgpeseg sgvpprpeea detwdskedk ihnaeniqpg | |
| | eqkyeyksdq wkplnleekk rydrefllgf qfifasmqkp eglphisdvv | |
| | ldkanktplr pldptrlqgi ncgpdftpsf anlgrttlst rgpprggpgg | |
| | elprgpaglg prrsqqgprk eprkiiatvl mtediklnka ekawkpsskr | |
| | taadkdrgee dadgsktqdl frrvrsilnk ltpqmfqqlm kqvtqlaidt | |
| | eerlkgvidl ifekaisepn fsvayanmcr clmalkvptt ekptvtvnfr | |
| | klllnrcqke fekdkdddev fekkqkemde aataeergrl keeleeardi | |
| | arrrslgnik figelfklkm lteaimhdcv vkllknhdee sleclcrllt | |
| | tigkdldfek akprmdqyfn qmekiikekk tssrirfmlq dvldlrgsnw | |
| | vprrgdqgpk tidqihkeae meehrehikv qqlmakgsdk rrggppgppi | |
| | srglplvddg gwntvpiskg srpidtsrlt kitkpgsids nnqlfapggr | |
| | lswgkgssgg sgakpsdaas eaarpatstl nrfsalqqav ptestdnrrv | |
| | vqrsslsrer gekagdrgdr lerserggdr gdrldrartp atkrsfskev | |
| | eersrerpsq peglrkaasl tedrdrgrda vkreaalppv splkaalsee | |
| | elekkskaii eeylhlndmk eavqcvqela spsllfifvr hgvestlers | |
| | aiarehmgql lhqllcaghl staqyyqgly eilelaedme idiphvwlyl | |
| | aelvtpilqe ggvpmgelfr eitkplrplg kaasllleil gllcksmgpk | |
| | kvgtlwreag lswkeflpeg qdigafvaeq kveytlgees eapgqralps | |
| | eelnrqlekl lkegssnqrv fdwieanlse qqivsntlvr almtavcysa | |
| | iifetplrvd vavlkarakl lqkylcdeqk elqalyalqa lvvtleqppn | |
| | llrmffdaly dedvvkedaf yswesskdpa eqqgkgvalk svtaffkwlr | |
| | eaeeesdhn | |
| 53 | agcagagtgg cgcagcggaa gcgtgctggg cccataaccc agaggtcgat | Human |
| | ggatcgaaac catcctctgc tacca | initiator Met- |
| | | tRNA-I |
| 54 | msaiqnlhsf dpfadaskgd dllpagtedy ihiriqqrng rktlttvqgi | Eukaryotic |
| | addydkkklv kafkkkfacn gtviehpeyg eviqlqgdqr knicqflvei | translation |
| | glakddqlkv hgf | initiation factor 1 |
| 55 | mpknkgkggk nrrrgknene sekrelvfke dgqeyaqvik | Eukaryotic |
| | mlgngrleam cfdgvrrlch irgklrkkvw intsdiilig lrdyqdnkad | translation |
| | vilkynadea rslkaygelp ehakinetdt fgpgdddeiq fddigddded | initiation factor |
| | iddi | 1A |
| 56 | mpglscrfyq hkfpevedvv mvnvrsiaem gayvslleyn niegmilise | Eukaryotic |
| | lsrrrirsin klirigrnec vvvirvdkek gyidlskrrv speeaikced | translation |
| | kftksktvys ilrhvaevle ytkdeqlesl fqrtawvfdd kykrpgygay | initiation factor |
| | dafkhavsdp sildsldlne derevlinni nrrltpqavk iradievacy | 2, subunit 1 |
| | gyegidavke alraglncst enmpikinli appryvmttt tlerteglsv | alpha [Homo |
| | lsqamavike kieekrgvfn vqmepkvvtd tdetelarqm erlerenaev | sapiens] |
| | dgdddaeeme akaed | |
| 57 | msgdemifdp tmskkkkkkk kpfmldeegd tqteetqpse tkevepepte | Eukaryotic |
| | dkdleadeed trkkdasddl ddlnffnqkk kkkktkkifd ideaeegvkd | translation |
| | lkiesdvqep tepeddldim lgnkkkkkkn vkfpdedeil ekdealeded | initiation factor |
| | nkkddgisfs nqtgpawags erdytyeell nrvfnimrek npdmvagekr | 2 beta [Homo |
| | kfvmkppqvv rvgtkktsfv nftdickllh rqpkhllafl laelgtsgsi | sapiens] |
| | dgnnqlvikg rfqqkqienv lrryikeyvt chtcrspdti lqkdtrlyfl | |
| | qcetchsrcs vasiktgfqa vtgkraqlra kan | |
| 58 | maggeagvtl gqphlsrqdl ttldvtkltp lshevisrqa tinigtighv | Eukaryotic |
| | ahgkstvvka isgvhtvrfk nelernitik lgyanakiyk lddpscprpe | translation |
| | cyrscgsstp defptdipgt kgnfklvrhv sfvdcpghdi lmatmlngaa | initiation factor |
| | vmdaalllia gnescpqpqt sehlaaieim klkhililqn kidlvkesqa | 2, subunit 3 |
| | keqyeqilaf vqgtvaegap iipisaqlky nievvceyiv kkipvpprdf | gamma [Homo |
| | tseprlivir sfdvnkpgce vddlkggvag gsilkgvlkv gqeievrpgi | sapiens] |
| | vskdsegklm ckpifskivs lfaehndlqy aapggligvg tkidptlcra | |
| | drmvgqvlga vgalpeifte leisyfllrr llgvrtegdk kaakvqklsk | |
| | nevlmvnigs lstggrvsav kadlgkivlt npvctevgek ialsrrvekh | |
| | wrligwgqir rgvtikptvd dd | |
| 59 | mpayfqrpen alkranefle vgkkqpaldv lydvmkskkh rtwqkihepi | Eukaryotic |
| | mlkylelcvd lrkshlakeg lyqyknicqq vniksledvv raylkmaeek | translation |
| | teaakeesqq mvldiedldn iqtpesvlls avsgedtqdr tdrllltpwv | initiation factor |
| | kflwesyrqc ldllrnnsrv erlyhdiaqq afkfclqytr kaefrklcdn | 3A [Homo |
| | lrmhlsqiqr hhnqstainl nnpesqsmhl etrlvqldsa ismelwqeaf | sapiens] |
| | kavedihglf slskkppkpq lmanyynkvs tvfwksgnal fhastlhrly | |
| | hlsremrknl tqdemqrmst rvllatlsip itpertdiar lldmdgiive | |
| | kqrrlatllg lqapptrigl indmvrfnvl qyvvpevkdl ynwlevefnp | |
| | lklcervtkv lnwvreqpek epelqqyvpq lqnntilrll qqvsqiyqsi | |
| | efsrltslvp fvdafqlera ivdaarhcdl qvridhtsrt lsfgsdlnya | |
| | tredapigph lqsmpseqir nqltamssvl akalevikpa hilqekeeqh | |
| | qlavtaylkn srkehqrila rrqtieerke rleslniqre keeleqreae | |
| | lqkvrkaeee rlrqeakere kerilqeheq ikkktvrerl eqikktelga | |
| | kafkdidied leeldpdfim akqveqleke kkelqerlkn qekkidyfer | |
| | akrleeipli ksayeeqrik dmdlweqqee erittmqler ekalehknrm | |
| | srmledrdlf vmrlkaarqs vyeeklkqfe erlaeerhnr leerkrqrke | |
| | errityyrek eeeeqrraee qmlkereere raerakreee lreyqervkk | |
| | leeverkkrq releieerer rreeerrlgd sslsrkdsrw gdrdsegtwr | |
| | kgpeadsewr rgppekewrr gegrdedrsh rrdeerprrl gddedrepsl | |
| | rpdddrvprr gmdddrgprr gpeedrfsrr gadddrpswr ntdddrpprr | |
| | iadedrgnwr hadddrpprr gldedrgswr tadedrgprr gmdddrgprr | |
| | ggadderssw rnadddrgpr rgldddrgpr rgmdddrgpr rgmdddrgpr | |
| | rgmdddrgpr rgldddrgpw rnadddripr rgaeddrgpw rnmdddrlsr | |
| | radddrfprr gddsrpgpwr plvkpggwre kekareeswg ppresrpsee | |
| | rewdrekerd rdnqdreend kdpererdre rdvdredrfr rprdeggwrr | |
| | gpaeessswr dssrrddrdr ddrrrerddr rdlrerrdlr ddrdrrgppl | |
| | rsereevssw rraddrkddr veerdpprrv pppalsrdre rdrdrerege | |
| | kekaswraek dreslrrtkn etdedgwttv rr | |
| 60 | mqdaenvavp eaaeeraepg qqqpaaeppp aegllrpagp gapeaagtea | Eukaryotic |
| | sseevgiaea gpesevrtep aaeaeaasgp sespsppaae elpgshaepp | translation |
| | vpaqgeapge qardersdsr aqavsedagg negraaeaep ralengdade | initiation factor |
| | psfsdpedfv ddvseeellg dvlkdrpqea dgidsvivvd nvpqvgpdrl | 3B [Homo |
| | eklknvihki fskfgkitnd fypeedgktk gyifleyasp ahavdavkna | sapiens] |
| | dgykldkqht frvnlftdfd kymtisdewd ipekqpfkdl gnlrywleea | |
| | ecrdqysvif esgdrtsifw ndvkdpvsie erarwtetyv rwspkgtyla | |
| | tfhqrgialw ggekfkqiqr fshqgvqlid fspcerylvt fsplmdtqdd | |
| | pqaiiiwdil tghkkrgfhc essahwpifk wshdgkffar mtldtlsiye | |
| | tpsmglldkk slkisgikdf swspggniia fwvpedkdip arvtlmqlpt | |
| | rqeirvrnlf nvvdcklhwq kngdylcvkv drtpkgtqgv vtnfeifrmr | |
| | ekqvpvdvve mketiiafaw epngskfavl hgeaprisvs fyhvknngki | |
| | elikmfdkqq antifwspqg qfvvlaglrs mngalafvdt sdctvmniae | |
| | hymasdvewd ptgryvvtsv swwshkvdna ywlwtfqgrl | |
| | lqknnkdrfc qllwrprppt llsqeqikqi kkdlkkyski feqkdrlsqs | |
| | kaskelverr rtmmedfrky rkmaqelyme qknerlelrg gvdtdeldsn | |
| | vddweeetie ffvteeiipl gnqe | |
| 61 | msrffttgsd sesesslsge elvtkpvggn ygkqplllse deedtkrvvr | Eukaryotic |
| | sakdkrfeel tnlirtirna mkirdvtkcl eefellgkay gkaksivdke | translation |
| | gvprfyiril adledylnel wedkegkkkm nknnakalst lrqkirkynr | initiation factor |
| | dfeshitsyk qnpeqsaded aekneedseg ssdedededg vsaatflkkk | 3C [Homo |
| | seapsgesrk flkkmddede dsedsedded wdtgstssds | sapiens] |
| | dseeeegkqt alasrflkka pttdedkkaa ekkredkakk khdrkskrld | |
| | eeeednegge wervrggvpl vkekpkmfak gteithavvi kklneilqar | |
| | gkkgtdraaq iellqllvqi aaennlgegv ivkikfniia slydynpnla | |
| | tymkpemwgk cldcinelmd ilfanpnifv genileesen lhnadqplrv | |
| | rgciltlver mdeeftkimq ntdphsqeyv ehlkdeaqvc | |
| | aiiervqryl eekgtteevc riyllrilht yykfdykahq rqltppegss | |
| | kseqdqaene gedsavlmer lckyiyakdr tdrirtcail chiyhhalhs | |
| | rwyqardlml mshlqdniqh adppvqilyn rtmvqlgica frqgltkdah | |
| | nalldiqssg rakellgqgl llrslqernq eqekverrrq vpfhlhinle | |
| | llecvylvsa mlleipymaa hesdarrrmi skqfhhqlrv gerqpllgpp | |
| | esmrehvvaa skamkmgdwk tchsfiinek mngkvwdlfp | |
| | eadkvrtmlv rkiqeeslrt ylftyssvyd sismetlsdm feldlptvhs | |
| | iiskmiinee lmasldqptq tvvmhrtept aqqnlalqla eklgslvenn | |
| | ervfdhkqgt yggyfrdqkd gyrknegymr rggyrqqqsq tay | |
| 62 | makfmtpviq dnpsgwgpca vpeqfrdmpy qpfskgdrlg | Eukaryotic |
| | kvadwtgaty qdkrytnkys sqfgggsqya yfheedessf qlvdtartqk | translation |
| | tayqrnrmrf aqrnlrrdkd rrnmlqfnlq ilpksakqke rerirlqkkf | initiation factor |
| | qkqfgvrqkw dqksqkprds svevrsdwev keemdfpqlm | 3D [Homo |
| | kmrylevsep qdieccgale yydkafdrit trsekplrsi krifhtvttt | sapiens] |
| | ddpvirklak tqgnvfatda ilatlmsctr svyswdivvq rvgsklffdk | |
| | rdnsdfdllt vsetaneppq degnsfnspr nlameatyin hnfsqqclrm | |
| | gkerynfpnp npfveddmdk neiasvayry rrwklgddid livrcehdgv | |
| | mtgangevsf iniktlnewd srhcngvdwr qkldsqrgav iatelknnsy | |
| | klarwtccal lagseylklg yvsryhvkds srhvilgtqq fkpnefasqi | |
| | nlsvenawgi lrcvidicmk leegkylilk dpnkqvirvy slpdgtfssd | |
| | edeeeeeeee eeeeeeet | |
| 63 | maeydlttri ahfldrhlvf plleflsvke iynekellqg kldllsdtnm | Eukaryotic |
| | vdfamdvykn lysddiphal rekrttvvaq lkqlqaetep ivkmfedpet | translation |
| | trqmqstrdg rmlfdyladk hgfrqeyldt lyryakfqye cgnysgaaey | initiation factor |
| | lyffrvlvpa tdrnalsslw gklaseilmq nwdaamedlt rlketidnns | 3E [Homo |
| | vssplqslqq rtwlihwslf vffnhpkgrd niidlflyqp qylnaiqtmc | sapiens] |
| | philryltta vitnkdvrkr rqvlkdlvkv iqqesytykd pitefvecly | |
| | vnfdfdgaqk klrecesvlv ndfflvacle dfienarlfi fetfcrihqc | |
| | isinmladkl nmtpeeaerw ivnlirnarl dakidsklgh vvmgnnavsp | |
| | yqqviektks lsfrsqmlam niekklnqns rseapnwatq dsgfy | |
| 64 | matpavpvsa ppatptpvpa aapasvpapt papaaapvpa aapasssdpa | Eukaryotic |
| | aaaaataapg qtpasaqapa qtpapalpgp alpgpfpggr vvrlhpvila | translation |
| | sivdsyerrn egaarvigtl lgtvdkhsve vtncfsvphn esedevavdm | initiation factor |
| | efaknmyelh kkvspnelil gwyatghdit ehsvliheyy sreapnpihl | 3F [Homo |
| | tvdtslqngr msikayvstl mgvpgrtmgv mftpltvkya yydterigvd | sapiens] |
| | limktcfspn rviglssdlq qvggasariq dalstvlqya edvlsgkvsa | |
| | dntvgrflms lvnqvpkivp ddfetmlnsn indllmvtyl anltqsqial | |
| | neklvnl | |
| 65 | mptgdfdskp swadqveeeg eddkcvtsel lkgiplatgd tspepellpg | Eukaryotic |
| | aplpppkevi ngniktvtey kidedgkkfk ivrtfrietr kaskavarrk | translation |
| | nwkkfgnsef dppgpnvatt tvsddvsmtf itskedlncq eeedpmnklk | initiation factor |
| | gqkivscric kgdhwttrcp ykdtlgpmqk elaeqlglst gekeklpgel | 3G [Homo |
| | epvqatqnkt gkyvppslrd gasrrgesmq pnrraddnat irvtnlsedt | sapiens] |
| | retdlqelfr pfgsisriyl akdkttgqsk gfafisfhrr edaaraiagv | |
| | sgfgydhlil nvewakpstn | |
| 66 | masrkegtgs tatsssstag aagkgkgkgg sgdsavkqvq idglvvlkii | Eukaryotic |
| | khyqeegqgt evvqgvllgl vvedrleitn cfpfpqhted dadfdevqyq | translation |
| | memmrslrhv nidhlhvgwy qstyygsfvt ralldsqfsy qhaieesvvl | initiation factor |
| | iydpiktaqg slslkayrlt pklmevckek dfspealkka nitfeymfee | 3H [Homo |
| | vpiviknshl invlmwelek ksavadkhel lslassnhlg knlqllmdrv | sapiens] |
| | demsqdivky ntymrntskq qqqkhqyqqr rqqenmqrqs | |
| | rgepplpeed lsklfkppqp parmdsllia gqintycqni keftaqnlgk | |
| | lfmaqalqey nn | |
| 67 | mkpillqghe rsitqikynr egdllftvak dpivnvwysv ngerlgtymg | Eukaryotic |
| | htgavwcvda dwdtkhvltg sadnscrlwd cetgkqlall ktnsavrtcg | translation |
| | fdfggniimf stdkqmgyqc fvsffdlrdp sqidnnepym kipcndskit | initiation factor |
| | savwgplgec iiaghesgel nqysaksgev lvnvkehsrq indiqlsrdm | 3I [Homo |
| | tmfvtaskdn taklfdsttl ehqktfrter pvnsaalspn ydhvvlgggq | sapiens] |
| | eamdvtttst rigkfearff hlafeeefgr vkghfgpins vafhpdgksy | |
| | ssggedgyvr ihyfdpqyfe fefea | |
| 68 | maaaaaaagd sdswdadafs vedpvrkvgg ggtaggdrwe | Eukaryotic |
| | gedededvkd nwdddddekk eeaevkpevk isekkkiaek | translation |
| | ikekerqqkk rqeeikkrle epeepkvltp eeqladklrl kklqeesdle | initiation factor |
| | laketfgvnn avygidamnp ssrddftefg kllkdkitqy ekslyyasfl | 3J [Homo |
| | evlvrdvcis leiddlkkit nsltvlcsek qkqekqskak kkkkgvvpgg | sapiens] |
| | glkatmkddl adyggydggy vqdyedfm | |
| 69 | mamfeqmran vgkllkgidr ynpenlatle ryvetqaken aydleanlav | Eukaryotic |
| | lklyqfnpaf fqttvtaqil lkaltnlpht dftlckcmid qahqeerpir | translation |
| | qilylgdlle tchfqafwqa ldenmdlleg itgfedsvrk fichvvgity | initiation factor |
| | qhidrwllae mlgdlsdsql kvwmskygws adesgqific sqeesikpkn | 3K [Homo |
| | ivekidfdsv ssimassq | sapiens] |
| 70 | msypaddyes eaaydpyayp sdydmhtgdp kqdlayerqy | Eukaryotic |
| | eqqtyqvipe viknfiqyfh ktvsdlidqk vyelqasrvs sdvidqkvye | translation |
| | iqdiyenswt klterffknt pwpeaeaiap qvgndavfli lykelyyrhi | initiation factor |
| | yakvsggpsl eqrfesyyny cnlfnyilna dgpaplelpn qwlwdiidef | 3L [Homo |
| | iyqfqsfsqy rcktakksee eidflrsnpk iwnvhsvlnv lhslvdksni | sapiens] |
| | nrqlevytsg gdpesvagey grhslykmlg yfslvgllrl hsllgdyyqa | |
| | ikvlenieln kksmysrvpe cqvttyyyvg faylmmrryq dairvfanil | |
| | lyiqrtksmf qrttykyemi nkqneqmhal laialtmypm ridesihlql | |
| | rekygdkmlr mqkgdpqvye elfsyscpkf lspvvpnydn | |
| | vhpnyhkepf lqqlkvfsde vqqqaqlsti rsflklyttm pvaklagfld | |
| | lteqefriql lvfkhkmknl vwtsgisald gefqsasevd fyidkdmihi | |
| | adtkvarryg dffirqihkf eelnrtlkkm gqrp | |
| 71 | msvpafidis eedqaaelra ylkskgaeis eensegglhv dlaqiieacd | Eukaryotic |
| | vclkeddkdv esvmnsvvsl llilepdkqe alieslcekl vkfregerps | translation |
| | lrlqllsnlf hgmdkntpvr ytvycslikv aascgaiqyi pteldqvrkw | initiation factor |
| | isdwnlttek khtllrllye alvdckksda askvmvellg sytednasqa | 3M [Homo |
| | rvdahrcivr alkdpnaflf dhlltlkpvk flegelihdl ltifvsakla | sapiens] |
| | syvkfyqnnk dfidslgllh eqnmakmrll tfmgmavenk eisfdtmqqe | |
| | lqigaddvea fvidavrtkm vyckidqtqr kvvvshsthr tfgkqqwqql | |
| | ydtlnawkqn lnkvknslls lsdt | |
| 72 | ggaauucaacgcagaguacgcggggcaacacugagaaguuaucuuaaggg | (NCF2) 5′- |
| | aggcugggccccauucuacucaucuggcccagaaagugaacaccuugggg | UTR |
| | gccacuaaggcagcccugcuaggggagacgcuccaaccugucuucucucu | |
| | gucuccuggcagcucucuuggccuccuaguuucuaccuaauccauggaag | |
| | acgccaaaaacauaaagaaaggcccggcgccauucuauccgcuggaagau | |
| | ggaaccgcuggagagcaacugcauaaggcuaugaagagauacgcccuggu | |
| | uccuggaacaauugcuuuuacagaugcacauaucgagguggacaucacuu | |
| | acgcugaguacuucgaaauguccguucgguuggcagaagcuaugaaacga | |
| | uaugggcugaauacaaaucacagaaucgucguaugcagugaaaacucucu | |
| | ucaauucuuuaugccgguguugggcgcguuauuuaucggaguugcaguug | |
| | cgcccgcgaacgacauuuauaaugaacgugaauugcucaacaguaugggc | |
| | auuucgcagccuaccgugguguucguuuccaaaaagggguugcaaaaaau | |
| | uuugaacgugcaaaaaaagcucccaaucauccaaaaaauuauuaucaugg | |
| | auucuaaaacggauuaccagggauuucagucgauguacacguucgucaca | |
| | ucucaucuaccucccgguuuuaaugaauacgauuuugugccagaguccuu | |
| | cgauagggacaagacaauugcacugaucaugaacuccucuggaucuacug | |
| | gucugccuaaaggugucgcucugccucauagaacugccugcgugagauuc | |
| | ucgcaugccagagauccuauuuuuggcaaucaaaucauuccggauacugc | |
| | gauuuuaaguguuguuccauuccaucacgguuuuggaauguuuacuacac | |
| | ucggauauuugauauguggauuucgagucgucuuaauguauagauuugaa | |
| | gaagagcuguuucugaggagccuucaggauuacaagauucaaagugcgcu | |
| | gcuggugccaacccuauucuccuucuucgccaaaagcacucugauugaca | |
| | aauacgauuuaucuaauuuacacgaaauugcuucugguggcgcuccccuc | |
| | ucuaaggaagucggggaagcgguugccaagagguuccaucugccagguau | |
| | caggcaaggauaugggcucacugagacuacaucagcuauucugauuacac | |
| | ccgagggggaugauaaaccgggcgcggucgguaaaguuguuccauuuuuu | |
| | gaagcgaagguuguggaucuggauaccgggaaaacgcugggcguuaauca | |
| | aagaggcgaacugugugugagagguccuaugauuauguccgguuauguaa | |
| | acaauccggaagcgaccaacgccuugauugacaaggauggauggcuacau | |
| | ucuggagacauagcuuacugggacgaagacgaacacuucuucaucguuga | |
| | ccgccugaagucucugauuaaguacaaaggcuaucagguggcucccgcug | |
| | aauuggaauccaucuugcuccaacaccccaacaucuucgacgcagguguc | |
| | gcaggucuucccgacgaugacgccggugaacuucccgccgccguuguugu | |
| | uuuggagcacggaaagacgaugacggaaaaagagaucguggauuacgucg | |
| | ccagucaaguaacaaccgcgaaaaaguugcgcggaggaguuguguuugug | |
| | gacgaaguaccgaaaggucuuaccggaaaacucgacgcaagaaaaaucag | |
| | agagauccucauaaaggccaagaagggcggaaagaucgccguguaauucu | |
| | agagucggggcggccggccgcuucgagcagacaugauaagauacauugau | |
| | gaguuuggacaaaccacaacuagaaugcagugaaaaaaaugcuuuauuug | |
| | ugaaauuugugaugcuauugcuuuauuuguaaccauuauaagcugcaaua | |
| | aacaaguuaacaacaacaauugcauucauuuuauguuucagguucagggg | |
| | gaggugugggagguuuuuuaaagcaaguaaaaccucuacaaaugugguaa | |
| | aaucgauaaggaucugaacgauggagcggagaaugggcggaacugggcgg | |
| | aguuaggggcgggaugggcggaguuaggggcgggacuaugguugcugacu | |
| | aauugagaugcaugcuuugcauacuucugccugcuggggagccuggggac | |
| | uuuccacaccugguugcugacuaauugagaugcaugcuuugcauacuucu | |
| | gccugcuggggagccuggggacuuuccacacccuaacugacacacauucc | |
| | acagcggauc | |
| 73 | gggcggccgcgaauucggucaacgccugcggcuguugauauucuugcuca | CDC25 mRNA |
| | gaggccguaacuuuggccuucugcucagggaagacucugaguccgacguu | |
| | ggccuacccagucggaaggcagagcugcaaucuaguuaacuaccuccuuu | |
| | ccccuagauuuccuuucauucugcucaagucuucgccuguguccgauccc | |
| | uaucuacuuucucuccucuuguaggcaagccucagacuccaggcuugagc | |
| | uagguuuuguuuuucuccuggugagaauucgaagaccaugucuacggaac | |
| | ucuucucauccacaagagaggaaggaagcucuggcucaggacccaguuuu | |
| | aggucuaaucaaaggaaaauguuaaaccugcuccuggagagagacacuuc | |
| | cuuuaccgucuguccagaugucccuagaacuccagugggcaaauuucuug | |
| | gugauucugcaaaccuaagcauuuugucuggaggaaccccaaaacguugc | |
| | cucgaucuuucgaaucuuagcaguggggagauaacugccacucagcuuac | |
| | cacuucugcagaccuugaugaaacuggucaccuggauucuucaggacuuc | |
| | aggaagugcauuuagcugggaugaaucaugaccagcaccuaaugaaaugu | |
| | agcccagcacagcuucuuuguagcacuccgaaugguuuggaccguggcca | |
| | uagaaagagagaugcaauguguaguucaucugcaaauaaagaaaaugaca | |
| | auggaaacuugguggacagugaaaugaaauauuugggcagucccauuacu | |
| | acuguuccaaaauuggauaaaaauccaaaccuaggagaagaccaggcaga | |
| | agagauuucagaugaauuaauggaguuuucccugaaagaucaagaagcaa | |
| | aggugagcagaaguggccuauaucgcuccccgucgaugccagagaacuug | |
| | aacaggccaagacugaagcagguggaaaaauucaaggacaacacaauacc | |
| | agauaaaguuaaaaaaaaguauuuuucuggccaaggaaagcucaggaagg | |
| | gcuuauguuuaaagaagacagucucucugugugacauuacuaucacucag | |
| | augcuggaggaagauucuaaccaggggcaccugauuggugauuuuuccaa | |
| | gguaugugcgcugccaaccgugucagggaaacaccaagaucugaaguaug | |
| | ucaacccagaaacaguggcugccuuacugucggggaaguuccagggucug | |
| | auugagaaguuuuaugucauugauugucgcuauccauaugaguaucuggg | |
| | aggacacauccagggagccuuaaacuuauauagucaggaagaacuguuua | |
| | acuucuuucugaagaagcccaucgucccuuuggacacccagaagagaaua | |
| | aucaucguguuccacugugaauucuccucagagaggggcccccgaaugug | |
| | ccgcugucugcgugaagaggacaggucucugaaccaguauccugcauugu | |
| | acuacccagagcuauauauccuuaaaggcggcuacagagacuucuuucca | |
| | gaauauauggaacugugugaaccacagagcuacugcccuaugcaucauca | |
| | ggaccacaagacugaguugcugaggugucgaagccagagcaaagugcagg | |
| | aaggggagcggcagcugcgggagcagauugcccuucuggugaaggacaug | |
| | agcccaugauaacauuccagccacuggcugcuaacaagucaccaaaagac | |
| | acugcagaaacccugagcagaaagaggccuucuggauggccaaacccaag | |
| | auuauuaaaagaugucucugcaaaccaacaggcuaccaacuuguauccag | |
| | gccugggaauggauuagguuucagcagagcugaaagcugguggcagaguc | |
| | cuggagcuggcucuauaaggcagccuugaguugcauagagauuuguauug | |
| | guucagggaacucuggcauuccuuuucccaacuccucaugucuucucaca | |
| | agccagccaacucuuucucucugggcuucgggcuaugcaagagcguuguc | |
| | uaccuucuuucuuuguauuuuccuucuuuguuucccccucuuucuuuuuu | |
| | aaaaauggaaaaauaaacacuacagaaugaggucga | |
| 74 | gcaacaacaacaacaacaacaacaacaacaacaacaacaacaagggcugc | CAA(Stem 3)- |
| | gggccuccgcagccccaacaacaaccaugguccguccuguagaaacccca | GUS mRNA |
| | acccgugaaaucaaaaaacucgacggccugugggcauucagucuggaucg | |
| | cgaaaacuguggaauugaucagcguuggugggaaagcgcguuacaagaaa | |
| | gccgggcaauugcugugccaggcaguuuuaacgaucaguucgccgaugca | |
| | auucguaauuaugcgggcaacgucugguaucagcgcgaagucuuuauacc | |
| | gaugaaagguugggcaggccagcguaucgugcugcguuucgaugcgguca | |
| | cucauuacggcaaagugugggucaauaaucaggaagugauggagcaucag | |
| | ggcggcuauacgccauuugaagccgaugucacgccguauguuauugccgg | |
| | gaaaaguguac | |
| 75 | gcaacaacaacaacaacaacaacaacaacaacaacaacaacaagggcugc | CAA(Stem 4)- |
| | gguggagccuuccaccgcagccccaacaacaaccaugguccguccuguag | GUS mRNA |
| | aaaccccaacccgugaaaucaaaaaacucgacggccugugggcauucagu | |
| | cuggaucgcgaaaacuguggaauugaucagcguuggugggaaagcgcguu | |
| | acaagaaagccgggcaauugcugugccaggcaguuuuaacgaucaguucg | |
| | ccgaugcaauucguaauuaugcgggcaacgucugguaucagcgcgaaguc | |
| | uuuauaccgaugaaagguugggcaggccagcguaucgugcugcguuucga | |
| | ugcggucacucauuacggcaaagugugggucaauaaucaggaagugaugg | |
| | agcaucagggcggcuauacgccauuugaagccgaugucacgccguauguu | |
| | auugccgggaaaaguguac | |
| 76 | gcaagaacaacaacaacaacaacaacaacaacaacaacaacaacaacaac | (CAA)n-GUS |
| | aacaacaacaacaacaccaugguccguccuguagaaaccccaacccguga | mRNA |
| | aaucaaaaaacucgacggccugugggcauucagucuggaucgcgaaaacu | |
| | guggaauugaucagcguuggugggaaagcgcguuacaagaaagccgggca | |
| | auugcugugccaggcaguuuuaacgaucaguucgccgaugcagauauucg | |
| | uaauuaugcgggcaacgucugguaucagcgcgaagucuuuauaccgaaag | |
| | guugggcaggccagcguaucgugcugcguuucgaugcggucacucauuac | |
| | ggcaaagugugggucaauaaucaggaagugauggagcaucagggcggcua | |
| | uacgccauuugaagccgaugucacgccguauguuauugccgggaaaagug | |
| | uac | |
| 77 | gcaacaacaacaacaacaacaacaacaacaacaacaacaacaagggcgcc | CAA(Stem 1)- |
| | ugccccaacaacaaccaugguccguccuguagaaaccccaacccgugaaa | GUS mRNA |
| | ucaaaaaacucgacggccugugggcauucagucuggaucgcgaaaacugu | |
| | ggaauugaucagcguuggugggaaagcgcguuacaagaaagccgggcaau | |
| | ugcugugccaggcaguuuuaacgaucaguucgccgaugcagauauucgua | |
| | auuaugcgggcaacgucugguaucagcgcgaagucuuuauaccgaaaggu | |
| | ugggcaggccagcguaucgugcugcguuucgaugcggucacucauuacgg | |
| | caaagugugggucaauaaucaggaagugauggagcaucagggcggcuaua | |
| | cgccauuugaagccgaugucacgccguauguuauugccgggaaaaguguac | |
| 78 | gcaacaacaacaacaacaacaacaacaacaacaacaacaacaagggcugc | CAA(Stem 2)- |
| | gccugcagccccaacaacaaccaugguccguccuguagaaaccccaaccc | GUS mRNA |
| | gugaaaucaaaaaacucgacggccugugggcauucagucuggaucgcgaa | |
| | aacuguggaauugaucagcguuggugggaaagcgcguuacaagaaagccg | |
| | ggcaauugcugugccaggcaguuuuaacgaucaguucgccgaugcagaua | |
| | uucguaauuaugcgggcaacgucugguaucagcgcgaagucuuuauaccg | |
| | aaagguugggcaggccagcguaucgugcugcguuucgaugcggucacuca | |
| | uuacggcaaagugugggucaauaaucaggaagugauggagcaucagggcg | |
| | gcuauacgccauuugaagccgaugucacgccguauguuauugccgggaaa | |
| | aguguac | |
| 79 | gcaacaacaacaacaacaacaacaacaacaacaacaacaacaacaaacca | (CAA)N- |
| | ugcaacaacaaaccaugguccguccuguagaaaccccaacccgugaaauc | AUG-AUG- |
| | aaaaaacucgacggccugugggcauucagucuggaucgcgaaaacugugg | GUS mRNA |
| | aauugaucagcguuggugggaaagcgcguuacaagaaagccgggcaauug | |
| | cugugccaggcaguuuuaacgaucaguucgccgaugcagauauucguaau | |
| | uaugcgggcaacgucugguaucagcgcgaagucuuuauaccgaaagguug | |
| | ggcaggccagcguaucgugcugcguuucgaugcggucacucauuacggca | |
| | aagugugggucaauaaucaggaagugauggagcaucagggcggcuauacg | |
| | ccauuugaagccgaugucacgccguauguuauugccgggaaaaguguac | |
| 80 | gggcgaauugggcccucuagaugcaugcucgagcggccgccagugugaug | CrPV IGR |
| | gauaucuauguuggcugaugagguuuacgacuucuaaaaagcaaaaaugu | IRES |
| | gaucuugcuuguaaauacaauuuugagagguuaauaaauuacaaguagug | |
| | cuauuuuuguauuuagguuagcuauuuagcuuuacguuccaggaugccua | |
| | guggcagccccacaauauccaggaagcccucucugcgguuuuucagauua | |
| | gguagucgaaaaaccuaagaaauuuaccugcuacauuucaagauaaacaa | |
| | gaaaauucacacauugaaaaugaagauaaaagacuuauguccgaacagaa | |
| | agaaauuguacauuuuguuagcgaaggaauuaccccuaguacuacugcgc | |
| | ucccugauaucguuaaucuuucaacuaauuaucuggacaugacuacgaga | |
| | gaagauagaauu | |
| 81 | gggagaccggaagcugucgacaagguuagcucuuucucguauacgauauu | CSFVΔ442.NS′ |
| | ggauacacuaaauuucgauuuggucuagggcaccccuccagcgacggccg | mRNA |
| | aaaugggcuagccaugcccauaguaggacuagcaaacggagggacuagcc | |
| | guaguggcgagcucccuggguggucuaaguccugaguacaggacagucgu | |
| | caguaguucgacgugagcacuagcccaccucgagaugcuacguggacgag | |
| | ggcaugcccaagacacaccuuaacccuggcgggggucgcuagggugaaau | |
| | cacauuaugugauggggguacgaccugauagggugcugcagaggcccacu | |
| | agcaggcuaguauaaaaaucucugcuguacauggcacauggaguugaauc | |
| | auuuugaauuauuauacaaaacaagcaaacaaaaaccagugggaguggag | |
| | gaaccggguaccauggaucccagcuuucagguagauugcuuucuuuggca | |
| | uguccgcaaacgaguugcagaccaagaacuaggugaugccccauuccuug | |
| | aucggcuucgccgagaucagaaaucccuaagaggaaggggcagcacucuu | |
| | ggucuggacaucgagacagccacacgugcuggaaagcagauaguggagcg | |
| | gauucugaaagaagaauccgaugaggcacuuaaaaugaccauggccucug | |
| | uaccugcgucgcguuaccuaaccgacaugacucuugaggaaaugucaagg | |
| | gaaugguccaugcucauacccaagcagaaaguggcaggcccucuuuguau | |
| | cagaauggaccaggcgaucauggauaaaaacaucauacugaaagcgaacu | |
| | ucagugugauuuuugaccggcuggagacucuaauauugcuaagggcuuuc | |
| | accgaagagggagcaauuguuggcgaaauuucaccauugccuucucuucc | |
| | aggacauacugcugaggaugucaaaaaugcaguuggaguccucaucggag | |
| | gacuugaauggaaugauaacacaguucgagucucugaaacucuacagaga | |
| | uucgcuuggagaagcaguaaugagaaugggagaccuccacucacuccaaa | |
| | acagaaacgagaaauggcgggaacaauuaggucagaaguuuugaagaaau | |
| | aagaugguugauugaagaagugagacacaaacugaagguaacagagaaua | |
| | guuuugagcaaauaacauuuaugcaagccuuacaucuauugcuugaagug | |
| | gagcaagagauaagaacuuucucauuucagcuuauuuaauaauaaaaaac | |
| | acccuuguuucuacugaaauu | |
| 82 | gggagaccggaagcugucgacuagccguaguggcgagcucccuggguggu | CSFV(128-442). |
| | cuaaguccugaguacaggacagucgucaguaguucgacgugagcacuagc | NS′ |
| | ccaccucgagaugcuacguggacgagggcaugcccaagacacaccuuaac | mRNA |
| | ccuggcgggggucgcuagggugaaaucacauuaugugauggggguacgac | |
| | cugauagggugcugcagaggcccacuagcaggcuaguauaaaaaucucug | |
| | cuguacauggcacauggaguugaaucauuuugaauuauuauacaaaacaa | |
| | gcaaacaaaaaccagugggaguggaggaaccggguaccauggaucccagc | |
| | uuucagguagauugcuuucuuuggcauguccgcaaacgaguugcagacca | |
| | agaacuaggugaugccccauuccuugaucggcuucgccgagaucagaaau | |
| | cccuaagaggaaggggcagcacucuuggucuggacaucgagacagccaca | |
| | cgugcuggaaagcagauaguggagcggauucugaaagaagaauccgauga | |
| | ggcacuuaaaaugaccauggccucuguaccugcgucgcguuaccuaaccg | |
| | acaugacucuugaggaaaugucaagggaaugguccaugcucauacccaag | |
| | cagaaaguggcaggcccucuuuguaucagaauggaccaggcgaucaugga | |
| | uaaaaacaucauacugaaagcgaacuucagugugauuuuugaccggcugg | |
| | agacucuaauauugcuaagggcuuucaccgaagagggagcaauuguuggc | |
| | gaaauuucaccauugccuucucuuccaggacauacugcugaggaugucaa | |
| | aaaugcaguuggaguccucaucggaggacuugaauggaaugauaacacag | |
| | uucgagucucugaaacucuacagagauucgcuuggagaagcaguaaugag | |
| | aaugggagaccuccacucacuccaaaacagaaacgagaaauggcgggaac | |
| | aauuaggucagaaguuuugaagaaauaagaugguugauugaagaagugag | |
| | acacaaacugaagguaacagagaauaguuuugagcaaauaacauuuaugc | |
| | aagccuuacaucuauugcuugaaguggagcaagagauaagaacuuucuca | |
| | uuucagcuuauuuaauaauaaaaaacacccuuguuucuacugaaauu | |

What is claimed is:

1. A method of forming a 48S complex comprising the use of a DExH-box protein.

2. The method of claim 1, wherein said DExH-box protein is DHX29 [Seq. ID No. 1].

3. The method of claim 2, further comprising the use of eukaryotic initiation factors.

4. The method of claim 3, wherein said eukaryotic initiation factors include eIF1 [Seq. ID No. 54], eIF1A [Seq. ID No. 55], eIF2 [Seq. ID Nos. 56, 57, 58], eIF3 [Seq. ID Nos. 59-71], eIF4A [Seq. ID No. 59], eIF4B [Seq. ID No. 50], eIF4F [Seq. ID Nos. 49, 51, 52], and combinations thereof.

5. The method of claim 2, wherein said 48S complex is formed on mRNAs containing longer stems.

6. The method of claim 5, wherein said mRNAs comprise long and structured 5′UTRs.

7. The method of claim 5, wherein said mRNAs comprise 5′-UTRs of 25 nt or more.

8. The method of claim 2, further comprising the use of 43S complexes.

9. The method of claim 8, wherein said DHX29 is present in substoichiometric amounts relative to 43S complexes.

10. A method of purifying a DExH-box protein comprising the steps of:
a. performing a ribosomal salt wash containing said DExH-box protein;
b. precipitating a first fraction of said ribosomal salt wash;
c. applying said first fraction to a DEAE column to provide an eluted fraction;
d. performing a step elution on a plurality of aliquots of said eluted fraction through a phosphocellulose column to provide a step-eluted fraction;
e. subjecting a step-eluted fraction to a first liquid chromatography column to provide a first purified fraction;
f. subjecting said first purified fraction to a second liquid chromatography column to provide a second purified fraction; and
g. applying said second purified fraction to a hydroxyapatite column to elute a substantially purified DExH-box protein.

11. The method of claim 10, wherein said substantially purified DExH-box protein is at least 95% pure.

12. The method of claim 10 wherein said DExH-box protein comprises DHX29 [Seq. ID No. 1].

13. The method of claim 10, wherein said step of precipitating said first fraction of said ribosomal salt wash comprises exposing said first fraction to ammonium sulfate.

14. A method of performing translation initiation during ribosomal scanning comprising the use of a DExH-box protein.

15. The method of claim 14 wherein said DExH-box protein comprises DHX29 [Seq. ID No. 1].

16. A method of using a DExH-box protein to achieve therapeutic regulation of gene expression.

17. The method of claim 16 wherein said DExH-box protein comprises DHX29 [Seq. ID No. 1].

18. A method of using a DExH-box protein as a biomarker for diagnosis of human cancer.

19. The method of claim 18 wherein said DExH-box protein comprises DHX29 [Seq. ID No. 1].
